SEQUENCE LISTING (107 sequences)

SEQ ID NO: 1 - protein, 119 aa, Homo sapiens
QVQLQQSGGGLVQPGGSLRLSCTASGFTFSSYAMSWVRQAPGKGLEWVSAISGSGGSTYYADSVKGRFTISRDNSKNTLYLQMNSLRVEDTAVYYCARYSIGRHTFDHWGQGTLVTVSA

SEQ ID NO: 2 - protein, 111 aa, Homo sapiens
NFMLTQPHSVSESPGKTVTISCTRSSGGIASNFVQWYQQRPGSVPTTVIYRDNQRPSGVPDRFSGSVDSSSNSASLTISGLKTDDEADYYCQSYDDHNHWVFGGGTKLTVL

SEQ ID NO: 3 - protein, 118 aa, Homo sapiens
KVQLVESGGGLVKPGGSLRLSCAASGFTFSNAWMNWVRQAPGKGLEWVGRIKRKTDGETTDYAAPVKGRFSISRDDSKNTLYLQMNSLKTEDTAVYYCAGSNRAFDIWGQGTMVTVSA

SEQ ID NO: 4 - protein, 113 aa, Homo sapiens
DIVMTQSPDSLAVSLGERATINCKSSQSVLYSSNNRNYLAWYQQKPGQPPKLLINQASTRASGVPDRFSGSGSGTEFTLIISSLHAEDVAIYYCQQYYTPPLAFGGGTKLEIK

SEQ ID NO: 5 - protein, 118 aa, Homo sapiens
QVQLQQSGGGLVQPGGSLRLSCAASGFTFSGYAMTWVRQAPGKGLEWVSAITSTGGRTYYADSVKGRFTTSRDNSRNTLYLQMNSLRAEDTAVYYCARESNFRAFDIWGQGTMVTVSA

SEQ ID NO: 6 - protein, 112 aa, Homo sapiens
EIVLTQSPLSLPVTPGEPASISCRSSQSLLHSNGYNYLDWYLQKPGQSPQLLIYLNSNRASGVPDRFSGSGSGTDFTLQISRVEAEDVGVYYCMQALQIPPTFGGGTKVDIK

SEQ ID NO: 7 - protein, 118 aa, Homo sapiens
EVQLVESGGGLVQPGGSLRLSCAASGFTFIDAWMTWVRQAPGKGLEWVSVIYSGGSTYYADSVKGRFTISRDNSKNTLYLQMNSLRAEDTAVYYCARGARGHPGQDYWGQGTLVTVSA

SEQ ID NO: 8 - protein, 112 aa, Homo sapiens
NFMLTQPHSVSESPGKTVTISCTRSSGTIASNFVQWYQQRPGSSPTPVIFENDRRPSGVPDRFSGSIDSSSNSASLTISSLNTEDKADYYCQSYDSSTHGWVFGGGTQLTVL

SEQ ID NO: 9 - protein, 117 aa, Homo sapiens
QVNLRESGGGLVQPGGSLRLSCAASGFTFSDYYMSWIRQAPGGGLEWVSYTSRFGSDTNYADSVKGRFTISRDNVQNSLYLQMNSLRAEDTAVYYCVRDVHNRDAYWGQGTLVTVSA

SEQ ID NO: 10 - protein, 110 aa, Homo sapiens
SYVLTQPPSASGTPGQRVTISCSGSSSNIGGNSVSWYQQLPGTAPKLLIYRNHQRPSGVPDRFSGSKSGTSASLAISGLRSDDEADYYCATWDFSLSGFVFGTGTKVTVL

SEQ ID NO: 11 - protein, 108 aa, Homo sapiens
QVQLVQSGGGLVQPGGSLRLSCAASGFTFSSYAMSWVRQAPGKGLEWVSAISGSGGSTYYADSVKGRFTISRDNSKNTYYCANTDYYDSSSHTPADYWGQGTLVTVSA

SEQ ID NO: 12 - protein, 108 aa, Homo sapiens
ETTLTQSPSSLSASVGDRVTITCRASQDIRNDLDWFQQKPGEAPKRLISAASNLQSGVPSRFSGGGSGSEFTLTIHSLESEDFATYYCQQSYITPPWTFGQGTKLEIK

SEQ ID NO: 13 - protein, 122 aa, Homo sapiens
QVQLQESGGGLVQPGGSLRLSCAASGFTFSSYGMSWVRQAPGKGLEWVSTISGSGSSTNYADSVKGRFTISRDNSKNTLFLQMNSLRAEDTAVYFCAKGRYYYDSLDAFDIWGQGTMVTVSA

SEQ ID NO: 14 - protein, 108 aa, Homo sapiens
EIVLTQSPGTLSLSPGERATLSCRASQEIRTAYLAWYQQKPGQAPRLLIYYASSRATGIPDRFSGSRSGTDFTLTISRLEPEDFAVYSCQQYDTSPPTFGQGTRLEIK

SEQ ID NO: 15 - protein, 122 aa, Homo sapiens
QVQLVESGGGLVQPGGSLRLSCAASGFTFSSYAMSWVRQAPGKGLEWVSAISGTGGSTYYADSVKGRFTISRDNSKNTLYLQMNSLRAEDTAAYYCAKDKWSSWPTYYFDYWGQGTLVTVSA

SEQ ID NO: 16 - protein, 110 aa, Homo sapiens
NFMLTQPHSVSESPGKTVTISCTRSSGSIASNYVQWYQQRPGSSPTTVIYEDNQRPSGVPDRFSGSIDSSSNSASLTISGLKTEDEADYYCQSYDSSNVIFGGGTKVTVL

SEQ ID NO: 17 - protein, 122 aa, Homo sapiens
QVQLQVSGGGLVQPGGSLRLSCAASGFTFSSYSMAWVRQAPGKGLEWVAAVSNSGVETYYADSVKGRFTISRDNSKNTLYLQMNSLRAEDTAVYYCAKRTRQLLTPREFDYWGQGTLVTVLA

SEQ ID NO: 18 - protein, 107 aa, Homo sapiens
ETTLTQSPSSVSASVGDRVTLTCRASQDITRWLAWYQQKPGKAPKLLIYDASSLQSGVPSRFSGSGSGTDFTLTISSLQPEDFATYYCQQGSSVPFTFGGGTKVEIK

SEQ ID NO: 19 - protein, 117 aa, Homo sapiens
QVNLRESGGGLIQPGGSLRLSCAASGFTFTNYAMSWVRQAPGKGLEWVSSVSSAGGSTYYADSVKGRFTISRDNAKNSLYLQMNSLRAEDTAVYYCARRVNRAFDLWGRGTLVTVSA

SEQ ID NO: 20 - protein, 110 aa, Homo sapiens
DVVMTQSPGTLSLSPGERATLSCRASQSVSSSYLAWYQQKPGQAPRLLIYGASSRATGIPDRFSGSGSGTDFTLTISRLEPEDFAVYYCQQYGSSPPMYTFGQGTKLEIK

SEQ ID NO: 21 - protein, 121 aa, Homo sapiens
QVQLQESGGGLVQPGGSLRLSCAASGFTFSNAWMSWVRQAPGKGLVWVGRIKSKTDGGTTDYAAPVKGRFTISRDDSKNTLYLQMNSLKTEDTAVYYCTTDKSYGYTFDYWGQGTLVTVSA

SEQ ID NO: 22 - protein, 110 aa, Homo sapiens
SYVLTQPPSASGTPGQRVTISCSGSGSNIGSNSVHWYQQLPGTAPKLLIYTNNQRPSGVPDRFSGSKSGISASLAISGLQSEDEAVYYCATWDDRLSGPVFAAGTKLTVL

SEQ ID NO: 23 - protein, 119 aa, Homo sapiens
QVNLRESGGGLVKPGGSLRLSCAASGFTFSSYWMHWVRQAPGKGLEWVSAISGSGAGTYYPDSVKGRFTISRDNSKNTLYLQMNSLRAEDTAVYYCARDRSLSFGFDIWGQGTLVSVSG

SEQ ID NO: 24 - protein, 110 aa, Homo sapiens
NFMLTQPHSVSGSPGKTVTISCTRSSGSIGSTYVQWYQQRPGSPPTTVIYKDDQRPSGVPDRFSGSIDGSSNSASLTISGLETEDEADYYCQSSDTSNLVFGGGTKVTVL

SEQ ID NO: 25 - protein, 121 aa, Homo sapiens
QVQLQQSGGGLVQPGGSLRLSCAAPGFTFSRYWMSWVRQAPGKGLEWVANIKGDGSQTYYADSVKGRFTISRDNAMKTVYLQMNSLRAEDTAIYYCAKGAAYHINSWLDPWGQGTLVTVSA

SEQ ID NO: 26 - protein, 108 aa, Homo sapiens
ETTLTQSPGTLSVSPGERVTLSCRASQSISGNYLAWYQQRPGQAPRLLIYGAFRRATGIPDRFSGSGSGTDFTLTITRLEPEDFATYYCQHYNNFPHTFGAGTKVDIK

SEQ ID NO: 27 - protein, 118 aa, Homo sapiens
KVQLVESGGGLVKPGGSLRLSCAASGITFKHAWMNWVRQAPGKGLEWVGRIKRKTDGETTDYAAPVKGRFSISRDDSKNTLYLQMNSLKTEDTAVYYCAGSNRAFDIWGQGTMVTVSA

SEQ ID NO: 28 - protein, 113 aa, Homo sapiens
DIVMTQSPDSLAVSLGERATINCKSSQSVLYQVNNRNYLAWYQQKPGQPPKLLINQASTRASGVPDRFSGSGSGTEFTLIISSLQAEDVAIYYCQQYYTPPLAFGGGTKLEIK

SEQ ID NO: 29 - protein, 118 aa, Homo sapiens
KVQLVESGGGLVKPGGSLRLSCAASGFTFNNAWMNWVRQAPGKGLEWVGRIKRKTDGETTDYAAPVKGRFSISRDDSKNTLYLQMNSLKTEDTAVYYCAGSNRAFDIWGQGTMVTVSA

SEQ ID NO: 30 - protein, 113 aa, Homo sapiens
DIVMTQSPDSLAVSLGERATINCKSSQTVLYPLNNRNYLAWYQQKPGQPPKLLINQASTRASGVPDRFSGSGSGTEFTLIISSLQAEDVAIYYCQQYYTPPLAFGGGTKLEIK

SEQ ID NO: 31 - protein, 118 aa, Homo sapiens
KVQLVESGGGLVKPGGSLRLSCAASGLTFERAWMNWVRQAPGKGLEWVGRIKRKTDGETTDYAAPVKGRFSISRDDSKNTLYLQMNSLKTEDTAVYYCAGSNRAFDIWGQGTMVTVSA

SEQ ID NO: 32 - protein, 113 aa, Homo sapiens
DIVMTQSPDSLAVSLGERATINCKSSQSVLYAGNNRNYLAWYQQKPGQPPKLLINQASTRASGVPDRFSGSGSGTEFTLIISSLQAEDVAIYYCQQYYTPPLAFGGGTKLEIK

SEQ ID NO: 33 - protein, 118 aa, Homo sapiens
KVQLVESGGGLVKPGGSLRLSCAASGFTFPNAWMNWVRQAPGKGLEWVGRIKRKTDGETTDYAAPVKGRFSISRDDSKNTLYLQMNSLKTEDTAVYYCAGSNRAFDIWGQGTMVTVSA

SEQ ID NO: 34 - protein, 113 aa, Homo sapiens
DIVMTQSPDSLAVSLGERATINCKSSQSVLYPGNNRNYLAWYQQKPGQPPKLLINQASTRASGVPDRFSGSGSGTEFTLIISSLQAEDVAIYYCQQYYTPPLAFGGGTKLEIK

SEQ ID NO: 35 - protein, 118 aa, Homo sapiens
KVQLVESGGGLVKPGGSLRLSCAASGLTFGNAWMNWVRQAPGKGLEWVGRIKRKTDGETTDYAAPVKGRFSISRDDSKNTLYLQMNSLKTEDTAVYYCAGSNRAFDIWGQGTMVTVSA

SEQ ID NO: 36 - protein, 113 aa, Homo sapiens; identical to SEQ ID NO: 34

SEQ ID NO: 37 - protein, 118 aa, Homo sapiens
KVQLVESGGGLVKPGGSLRLSCAASGFTFSNAWMNWVRQAPGKGLEWVGRIKRKTDGETTDYAAPVKGRFSISRDDSKNTLYLQMNSLKTEDTAVYYCAGGNHSSDIWGQGTMVTVSA

SEQ ID NO: 38 - protein, 113 aa, Homo sapiens
DIVMTQSPDSLAVSLGERATINCKSSQSVLYSSNNRNYLAWYQQKPGQPPKLLINQASTRASGVPDRFSGSGSGTEFTLIISSLQAEDVAIYYCQQYYTPPLAFGGGTKLEIK

SEQ ID NO: 39 - protein, 118 aa, Homo sapiens
KVQLVESGGGLVKPGGSLRLSCAASGFTFSNAWMNWVRQAPGKGLEWVGRIKRKTDGETTDYAAPVKGRFSISRDDSKNTLYLQMNSLKTEDTAVYYCAGGAHSSDIWGQGTMVTVSA

SEQ ID NO: 40 - protein, 113 aa, Homo sapiens; identical to SEQ ID NO: 38

SEQ ID NO: 41 - protein, 118 aa, Homo sapiens
KVQLVESGGGLVKPGGSLRLSCAASGFTFSNAWMNWVRQAPGKGLEWVGRIKRKTDGETTDYAAPVKGRFSISRDDSKNTLYLQMNSLKTEDTAVYYCAGGQHSSDIWGQGTMVTVSA

SEQ ID NO: 42 - protein, 113 aa, Homo sapiens; identical to SEQ ID NO: 38

SEQ ID NO: 43 - protein, 118 aa, Homo sapiens
KVQLVESGGGLVKPGGSLRLSCAASGFTFSNAWMNWVRQAPGKGLEWVGRIKRKTDGETTDYAAPVKGRFSISRDDSKNTLYLQMNSLKTEDTAVYYCAGSAYAFDAWGQGTMVTVSA

SEQ ID NO: 44 - protein, 113 aa, Homo sapiens; identical to SEQ ID NO: 38

SEQ ID NO: 45 - protein, 118 aa, Homo sapiens
KVQLVESGGGLVKPGGSLRLSCAASGFTFSNAWMNWVRQAPGKGLEWVGRIKRKTDGETTDYAAPVKGRFSISRDDSKNTLYLQMNSLKTEDTAVYYCAGSAYAFDSWGQGTMVTVSA

SEQ ID NO: 46 - protein, 113 aa, Homo sapiens; identical to SEQ ID NO: 38

SEQ ID NO: 47 - protein, 118 aa, Homo sapiens
KVQLVESGGGLVKPGGSLRLSCAASGFTFSNAWMNWVRQAPGKGLEWVGRIKRKTDGETTDYAAPVKGRFSISRDDSKNTLYLQMNSLKTEDTAVYYCAGSDRASDKWGQGTMVTVSA

SEQ ID NO: 48 - protein, 113 aa, Homo sapiens; identical to SEQ ID NO: 38

SEQ ID NO: 49 - protein, 118 aa, Homo sapiens
KVQLVESGGGLVKPGGSLRLSCAASGFTFSNAWMNWVRQAPGKGLEWVGRIKRKTDGETTDYAAPVKGRFSISRDDSKNTLYLQMNSLKTEDTAVYYCAGSAYAFDTWGQGTMVTVSA

SEQ ID NO: 50 - protein, 113 aa, Homo sapiens; identical to SEQ ID NO: 38

SEQ ID NO: 51 - protein, 118 aa, Homo sapiens
KVQLVESGGGLVKPGGSLRLSCAASGFTFSNAWMNWVRQAPGKGLEWVGRIKRKTDGETTDYAAPVKGRFSISRDDSKNTLYLQMNSLKTEDTAVYYCAGGNHSQDIWGQGTMVTVSA

SEQ ID NO: 52 - protein, 113 aa, Homo sapiens; identical to SEQ ID NO: 38

SEQ ID NO: 53 - protein, 118 aa, Homo sapiens
KVQLVESGGGLVKPGGSLRLSCAASGFTFSNAWMNWVRQAPGKGLEWVGRIKRKTDGETTDYAAPVKGRFSISRDDSKNTLYLQMNSLKTEDTAVYYCAGGQHSQDIWGQGTMVTVSA

SEQ ID NO: 54 - protein, 113 aa, Homo sapiens; identical to SEQ ID NO: 38

SEQ ID NO: 55 - protein, 118 aa, Homo sapiens
KVQLVESGGGLVKPGGSLRLSCAASGFTFSNAWMNWVRQAPGKGLEWVGRIKRKTDGETTDYAAPVKGRFSISRDDSKNTLYLQMNSLKTEDTAVYYCAGGAHSQDIWGQGTMVTVSA

SEQ ID NO: 56 - protein, 113 aa, Homo sapiens; identical to SEQ ID NO: 38

SEQ ID NO: 57 - protein, 118 aa, Homo sapiens; identical to SEQ ID NO: 3

SEQ ID NO: 58 - protein, 113 aa, Homo sapiens
DIVMTQSPDSLAVSLGERATINCKSSQSVLYSSNNRNYLAWYQQKPGQPPKLLINQASTRASGVPDRFSGSGSGTEFTLIISSLQAEDVAIYYCQQYLTPPLAFGGGTKLEIK

SEQ ID NO: 59 - protein, 118 aa, Homo sapiens; identical to SEQ ID NO: 3

SEQ ID NO: 60 - protein, 113 aa, Homo sapiens
DIVMTQSPDSLAVSLGERATINCKSSQSVLYSSNNRNYLAWYQQKPGQPPKLLINQASTRASGVPDRFSGSGSGTEFTLIISSLQAEDVAIYYCQQYLRPPLNFGGGTKLEIK

SEQ ID NO: 61 - protein, 118 aa, Homo sapiens; identical to SEQ ID NO: 3

SEQ ID NO: 62 - protein, 113 aa, Homo sapiens
DIVMTQSPDSLAVSLGERATINCKSSQSVLYSSNNRNYLAWYQQKPGQPPKLLINQASTRASGVPDRFSGSGSGTEFTLIISSLQAEDVAIYYCQQYLTPPLNFGGGTKLEIK

SEQ ID NO: 63 - protein, 118 aa, Homo sapiens; identical to SEQ ID NO: 3

SEQ ID NO: 64 - protein, 113 aa, Homo sapiens
DIVMTQSPDSLAVSLGERATINCKSSQSVLYSSNNRNYLAWYQQKPGQPPKLLINQASTRASGVPDRFSGSGSGTEFTLIISSLQAEDVAIYYCQNYLTPPLSFGGGTKLEIK

SEQ ID NO: 65 - protein, 118 aa, Homo sapiens; identical to SEQ ID NO: 3

SEQ ID NO: 66 - protein, 113 aa, Homo sapiens
DIVMTQSPDSLAVSLGERATINCKSSQSVLYSSNNRNYLAWYQQKPGQPPKLLINQASTRASGVPDRFSGSGSGTEFTLIISSLQAEDVAIYYCQQYLKAPLAFGGGTKLEIK

SEQ ID NO: 67 - protein, 118 aa, Homo sapiens; identical to SEQ ID NO: 3

SEQ ID NO: 68 - protein, 113 aa, Homo sapiens
DIVMTQSPDSLAVSLGERATINCKSSQSVLYSSNNRNYLAWYQQKPGQPPKLLINQASTRASGVPDRFSGSGSGTEFTLIISSLQAEDVAIYYCQQYLNAPLHFGGGTKLEIK

SEQ ID NO: 69 - protein, 118 aa, Homo sapiens; identical to SEQ ID NO: 3

SEQ ID NO: 70 - protein, 113 aa, Homo sapiens
DIVMTQSPDSLAVSLGERATINCKSSQSVLYSSNNRNYLAWYQQKPGQPPKLLINQASTRASGVPDRFSGSGSGTEFTLIISSLQAEDVAIYYCQQYLEAPLVFGGGTKLEIK

SEQ ID NO: 71 - protein, 118 aa, Homo sapiens; identical to SEQ ID NO: 3

SEQ ID NO: 72 - protein, 113 aa, Homo sapiens
DIVMTQSPDSLAVSLGERATINCKSSQSVLYSSNNRNYLAWYQQKPGQPPKLLINQASTRASGVPDRFSGSGSGTEFTLIISSLQAEDVAIYYCQQYLKAPLHFGGGTKLEIK

SEQ ID NO: 73 - protein, 118 aa, Homo sapiens; identical to SEQ ID NO: 3

SEQ ID NO: 74 - protein, 113 aa, Homo sapiens
DIVMTQSPDSLAVSLGERATINCKSSQSVLYSSNNRNYLAWYQQKPGQPPKLLINQASTRASGVPDRFSGSGSGTEFTLIISSLQAEDVAIYYCQRLIAPPFTFGGGTKLEIK

SEQ ID NO: 75 - protein, 118 aa, Homo sapiens; identical to SEQ ID NO: 3

SEQ ID NO: 76 - protein, 113 aa, Homo sapiens
DIVMTQSPDSLAVSLGERATINCKSSQSVLYSSNNRNYLAWYQQKPGQPPKLLINQASTRASGVPDRFSGSGSGTEFTLIISSLQAEDVAIYYCQNYLTPPLAFGGGTKLEIK

SEQ ID NO: 77 - DNA, 360 bp, Homo sapiens
caggtccaactggtgcagtctggggctgaagtgaagaagcctgggtcttcagtgaaggtgtcctgcaaggcttctggctacaccttcagcagctactatatgcactgggtgaggcaggctcctggacaaggccttgagtggatgggagagattaatcccaacaatgcccgtattaacttcaatgaaaagttcaagaccagggtcacactcactgtggacaaatccaccagcacagcatacatggagctcagcagcctgagatctgaggacaccgcggtctattactgtaccagaggatactataggtacggggcctggtttggttactggggccaagggactctggtcactgtctcttca

SEQ ID NO: 78 - DNA, 321 bp, Homo sapiens
gatatccagatgacacagtctccatcctccctgtctgcctctgtgggagacagagtcaccatcacttgcagggcaagtcaggacattagcgattatttgaactggtatcaacagaaaccaggcaaggctcctaaactcctgatctactacatatcaagattacactcaggagtcccatcacgcttcagtggcagtgggtctggaacagattatactctcaccattagctccctgcagccagaagattttgccacttactattgccaacagggtcatacacttccgtggaccttcggtggaggcaccaaggtggaaatcaaa

SEQ ID NO: 79 - DNA, 357 bp, Homo sapiens
caggtacagctgcagcagtcagggggaggcttggtacagcctggggggtccctaagactctcctgtacagcctctggattcacctttagcagctatgccatgagctgggtccgccaggctccagggaaggggctggagtgggtctcagcaattagtggtagtggtggtagcacatactacgcagactccgtgaagggccggttcaccatctccagagacaattccaagaacacgctgtatctgcaaatgaacagcctgagagtcgaggacacggccgtatattactgtgcgagatatagtattggtagacacacctttgaccactggggccagggcaccctggtcaccgtctcggcc

SEQ ID NO: 80 - DNA, 354 bp, Homo sapiens
aaggtgcagctggtggagtctgggggaggcttggtaaagcctggggggtcccttagactctcctgtgcagcctctggtttcactttcagtaacgcctggatgaactgggtccgccaggctccagggaaggggctggagtgggtcggccgtattaaaaggaaaactgatggtgagacaacagactacgctgcacccgtgaaaggcagattcagcatctcaagagatgattcaaaaaacaccctgtatctgcaaatgaacagcttgaaaaccgaggacacagccgtgtattactgcgctggcagtaaccgagcttttgatatctggggccaagggacaatggtcaccgtctctgcc

SEQ ID NO: 81 - DNA, 354 bp, Homo sapiens
caggtacagctgcagcagtcagggggaggcttggtacagcctggggggtccctgagactctcctgtgcagcctctggattcacctttagcggctatgccatgacttgggtccgccaggctccagggaaggggctggagtgggtctcagctattacttctactggtggtcgcacatactacgcagactccgtgaagggccggttcaccacctccagagacaattccaggaacacgttgtatctgcaaatgaacagcctgagagccgaagacacggccgtatattactgtgcgagagagtcaaacttcagggcttttgatatctggggccaagggacaatggtcaccgtctctgcc

SEQ ID NO: 82 - DNA, 354 bp, Homo sapiens
gaggtgcagctggtggagtctgggggaggcttggtacagcctggggggtcccttagactctcctgtgcagcctctggattcactttcattgacgcctggatgacctgggtccgccaggctccagggaaggggctggagtgggtctcagttatttatagcggtggtagcacatactacgcagactccgtgaagggcagattcaccatctccagagacaattccaagaacacgctgtatctgcaaatgaacagcctgagagccgaggacacggccgtatattactgtgcgagaggggctaggggccatcccgggcaggactactgggggcagggcaccctggtcaccgtctcggcc

SEQ ID NO: 83 - DNA, 351 bp, Homo sapiens
caggtcaacttaagggagtctgggggaggcttggtacagcctggcgggtccctgagactctcctgtgcagcctctggattcaccttcagtgactactacatgagctggatccgccaggctccagggggggggctggagtgggtttcatacactagtcgttttggtagtgacacaaactacgcagactctgtgaagggccgattcaccatctccagagacaacgtccagaactcactatatctgcaaatgaacagcctgagggccgaggacacggctgtttattactgtgtgagagatgtacataacagggatgcctactggggccagggcaccctggtcaccgtctcggcc

SEQ ID NO: 84 - DNA, 369 bp, Homo sapiens
caggtgcagctggtgcagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaacacagattactatgatagtagtagccatacccccgctgactactggggccagggcaccctggtcaccgtctcggcc

SEQ ID NO: 85 - DNA, 366 bp, Homo sapiens
caggtgcagctgcaggagtcggggggaggcttggtacagcctggggggtccctgagactctcctgtgcagcctctggattcacctttagtagctatggcatgagctgggtccgccaggctccagggaaagggctggagtgggtctcaactatcagtggcagtggtagtagcacaaactacgcagactccgtgaagggccggttcaccatctccagagacaattccaagaacacgctatttctgcaaatgaacagcctgagagccgaggacacggccgtatatttctgtgcgaaaggccgatattactatgatagtcttgatgcttttgatatctggggccaagggacaatggtcaccgtctctgcc

SEQ ID NO: 86 - DNA, 366 bp, Homo sapiens
caggtacagctggtggagtctgggggaggcttggtacagcctggggggtccctgagactctcctgtgcagcctctggattcacctttagcagctatgccatgagctgggtccgccaggctccagggaaggggctggagtgggtctcagctattagtggtactggtggtagcacatactacgcagactccgtgaagggccggttcaccatctccagagacaattccaagaacacgctgtatctgcaaatgaacagcctgagagccgaggacacggccgcatattactgtgcgaaagataaatggagcagctggcccacttactactttgactactggggccagggaaccctggtcaccgtctcggcc

SEQ ID NO: 87 - DNA, 366 bp, Homo sapiens
caggtgcagctgcaggtgtcggggggaggcttggtacagcctggggggtccctgagactctcctgtgcagcctctggattcaccttcagtagctatagcatggcctgggtccgccaggctccagggaaggggctggagtgggtcgcggctgttagtaatagtggtgttgagacatactacgcagactccgtgaagggccggttcaccatctccagagacaattccaagaacacgctgtatttgcaaatgaacagcctgagagccgaggacacggccgtatattactgtgcgaaacgaactagacaactgctaactccgcgggagtttgactactggggccagggcaccctggtcaccgtcttggcc

SEQ ID NO: 88 - DNA, 351 bp, Homo sapiens
caggtcaacttaagggagtctgggggaggcttgatacagcctggggggtccctgagactctcctgtgcagcctctggattcacctttaccaattatgccatgagctgggtccgccaggctccagggaaggggctggagtgggtctcaagtgttagtagtgctggtggtagtacatactacgcagactccgtgaagggccggttcaccatctccagagacaacgccaagaactcactgtatctgcaaatgaacagcctgagagccgaggacacggctgtttattactgtgcgagacgagtcaatcgggccttcgatctctggggccgtggaaccctggtcaccgtctcggcc

SEQ ID NO: 89 - DNA, 363 bp, Homo sapiens
caggtgcagctgcaggagtcggggggaggcttggtacagcctggggggtcccttagactctcctgtgcagcctctggattcactttcagtaacgcctggatgagctgggtccgccaggctccagggaaggggctggtgtgggttggccgtattaaaagcaaaactgatggtgggacaacagactacgctgcacccgtgaaaggcagattcaccatctcaagagatgattcaaaaaacacgctgtatctgcaaatgaacagcctgaaaaccgaggacacagccgtgtattactgtaccacagataagagctatggttacacatttgactactggggccagggcaccctggtcaccgtctcggcc

SEQ ID NO: 90 - DNA, 357 bp, Homo sapiens
caggtcaacttaagggagtctgggggaggcttggtaaagccgggggggtcccttagactctcctgtgcagcctctggattcaccttcagtagctactggatgcactgggtccgccaagccccagggaaggggctggagtgggtctcagctatcagtggtagtggtgccggcacatactacccagactccgtgaagggccggttcaccatctccagagacaattccaagaacacgctgtatctgcaaatgaacagcctgagagccgaggacacggccgtatattactgtgcgagagatcggtccttatcttttggttttgatatttggggccaaggcaccctggtctccgtctctggc

SEQ ID NO: 91 - DNA, 363 bp, Homo sapiens
caggtacagctgcagcagtcagggggaggcttggtccagccgggggggtcactgagactctcctgtgcagcccctggattcacctttagtagatattggatgagttgggtccgccaggctccagggaagggactggagtgggtggccaacataaagggagatggaagtcagacatactatgcggactctgtgaagggccgattcaccatctccagagacaacgccatgaaaacagtgtatctgcaaatgaacagcctgagagccgaggacacggccatatattactgtgcgaaaggggctgcttatcacattaacagctggctcgacccctggggccagggcaccctggtcaccgtctcggcc

SEQ ID NO: 92 - DNA, 333 bp, Homo sapiens
aattttatgctgactcagccccactctgtgtcggagtctccggggaagacggtaaccatctcctgcacccgcagcagtggcggcattgccagtaactttgtgcagtggtaccagcagcgcccgggcagtgtccccaccactgtgatctatagggataaccaaagaccctctggagtccctgatcggttctctggctccgtcgacagctcctccaattctgcctccctcaccatctctgggctgaagactgacgatgaggctgactattattgtcagtcctatgatgaccacaatcattgggtgttcggcggcgggaccaagctgaccgtccta

SEQ ID NO: 93 - DNA, 339 bp, Homo sapiens
gacatcgtgatgacccagtctccagactccctggctgtgtctctgggcgagagggccaccatcaactgcaagtccagccagagtgttttatacagctccaacaataggaactacctagcttggtaccagcagaaaccaggacagcctcctaagctgctcattaaccaggcatctacccgggcatccggcgtccctgaccgattcagtggcagcgggtctgggacagagttcactctcatcatcagcagcctgcaggctgaagatgtggcgatttattactgtcagcaatattatactcctcccctcgctttcggcggagggaccaagctggagatcaaa

SEQ ID NO: 94 - DNA, 336 bp, Homo sapiens
gaaattgtgttgacgcagtctccactctccctgcccgtcacccctggagagccggcctccatctcctgcaggtctagtcagagcctcctgcacagtaatggatacaactatttggattggtacctgcagaaaccagggcagtctccacagctcctgatctatttgaattctaatcgggcctccggggtccctgacaggttcagtggcagtggatcaggtacagattttacactgcaaatcagcagagtggaggctgaggatgttggggtttactactgtatgcaagctctacaaattcctcccactttcggcggagggaccaaagtggatatcaaa

SEQ ID NO: 95 - DNA, 336 bp, Homo sapiens
aattttatgctgactcagccccactctgtgtcggagtctccgggaaagacggtaaccatctcctgcacccgcagcagtggcaccattgccagcaactttgtgcagtggtatcaacagcgcccgggcagttcgcccaccccagtgatctttgagaatgaccgaagaccctctggggtccctgatcggttctctggctccatcgacagctcctccaattctgcctccctcaccatttcgtcactgaacactgaggacaaggctgactactactgtcagtcctatgatagcagcactcatgggtgggtgtttggcggagggacccagctcaccgtttta

SEQ ID NO: 96 - DNA, 330 bp, Homo sapiens
tcctatgtgctgactcagccaccctcagcgtctgggacccccgggcagagggtcaccatctcttgttctggaagcagctccaacatcggcggtaattctgtatcctggtaccagcaactcccaggaacggcccccaagctcctcatctataggaatcatcagcggccctcaggggtccctgaccgattctctggctccaagtctggcacctcagcctccctggccatcagtgggctccggtccgacgacgaggctgattattattgtgcaacatgggatttcagcctgagtggttttgtcttcggaactgggaccaaggtcaccgtccta

SEQ ID NO: 97 - DNA, 324 bp, Homo sapiens
gaaactacactcacgcagtctccatcctccctgtctgcatctgtaggagacagagtcaccatcacttgccgggccagtcaggacatcagaaatgatttagactggtttcaacaaaaaccaggcgaagcccctaaacgcctgatctctgctgcatctaatttgcagagtggggtcccctcacgattcagcggcggtggctctggctccgaattcactctcacaatccacagcctggagtctgaagattttgcaacttactactgtcaacagagttacattacccctccttggacgttcggccaagggaccaagctggagatcaaa

SEQ ID NO: 98 - DNA, 324 bp, Homo sapiens
gaaattgtgttgacgcagtctccaggcaccctgtctttgtctccaggggaaagagccaccctctcctgcagggccagtcaggaaattaggaccgcctacttagcctggtaccagcagaaacctggccaggctcccaggctcctcatctattatgcatccagcagggccactggcatcccagacaggttcagcggcagtaggtctgggacagacttcactctcaccatcagcagactggagcctgaagattttgcagtgtattcctgtcagcagtatgatacctcacctcccaccttcggccaagggacacgactggagattaaa

SEQ ID NO: 99 - DNA, 330 bp, Homo sapiens
aattttatgctgactcagccccactctgtgtcggagtctccggggaagacggtaaccatctcctgcacccgcagcagtggcagcattgccagcaactatgtgcagtggtaccaacagcgcccgggcagttcccccaccactgtgatctatgaggataaccaaagaccctctggggtccctgatcggttctctggctccatcgacagctcctccaactctgcctccctcaccatctctggactgaagactgaggacgaggctgactactactgtcagtcttatgatagcagcaatgtgatattcggcggagggaccaaggtcaccgtccta

SEQ ID NO: 100 - DNA, 321 bp, Homo sapiens
gaaactacactcacgcagtctccatcttccgtgtctgcatctgtaggagacagagtcaccctcacttgtcgggcgagtcaggatatcacaaggtggttagcctggtatcagcagaaaccagggaaagcccctaagctcctgatctatgatgcatccagtttgcaaagtggggtcccatcaagattcagcggcagtggatctgggacagatttcactctcaccatcagcagtctgcaacctgaagattttgcaacttactactgtcaacagggttccagtgttcctttcactttcggcggagggaccaaggtggagatcaaa

SEQ ID NO: 101 - DNA, 330 bp, Homo sapiens
gatgttgtgatgactcagtctccaggcaccctgtctttgtctccaggggaaagagccaccctctcctgcagggccagtcagagtgttagcagcagctacttagcctggtaccaacagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctcctatgtacacttttggccaggggaccaagctggagatcaaa

SEQ ID NO: 102 - DNA, 330 bp, Homo sapiens
tcctatgtgctgactcagccaccctcagcgtctgggacccccgggcagagggtcaccatctcttgttctggaagtggctccaacatcggaagtaattctgttcactggtaccagcaactcccaggaacggcccccaaactcctcatctatactaataatcagcggccctcaggggtccctgaccgattctctggctccaagtctggcatttcagcctccctggctatcagtgggctccagtctgaggatgaggctgtttattactgtgcaacgtgggatgacagactgagtggtccggtgttcgccgcagggaccaagctgaccgtccta

SEQ ID NO: 103 - DNA, 330 bp, Homo sapiens
aattttatgctgactcagccccactctgtgtcggggtctccggggaagacggtaaccatctcctgcacccgcagcagtggcagcattggcagcacctatgtgcagtggtaccaacagcgcccgggcagtccccccaccactgtgatctataaggatgaccaaagaccctctggggtccctgatcggttctctggctccatcgacggctcctccaactctgcctccctcaccatctctggactggagactgaggacgaggctgactactactgtcagtcttctgataccagcaatctggtcttcggcggagggaccaaggtcaccgtccta

SEQ ID NO: 104 - DNA, 324 bp, Homo sapiens
gaaactacactcacgcagtctccaggcaccctgtctgtttctccgggggaaagagttaccctctcctgcagggccagtcagagtattagcggtaattacttagcctggtaccagcagagacctggccaggctcccaggctcctcatctatggggcattcaggagggccactggcatcccagacaggttcagtggcagtgggtctgggacagacttcactctcaccatcaccagactggagcctgaagattttgcaacttattactgccaacactataataatttcccccacactttcggcgcagggaccaaagtggatatcaaa

SEQ ID NO: 105 - protein, 120 aa, Homo sapiens
QVQLVQSGAEVKKPGSSVKVSCKASGYTFSSYYMHWVRQAPGQGLEWMGEINPNNARINFNEKFKTRVTLTVDKSTSTAYMELSSLRSEDTAVYYCTRGYYRYGAWFGYWGQGTLVTVSS

SEQ ID NO: 106 - protein, 107 aa, Homo sapiens
DIQMTQSPSSLSASVGDRVTITCRASQDISDYLNWYQQKPGKAPKLLIYYISRLHSGVPSRFSGSGSGTDYTLTISSLQPEDFATYYCQQGHTLPWTFGGGTKVEIK

SEQ ID NO: 107 - protein, 123 aa, Homo sapiens
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNE

(19) United States
(12) Patent Application Publication
(10) Pub. No.: US 2021/0269522 A1
(43) Pub. Date: Sep. 2, 2021

(54) Novel CD47 Antibodies and Methods of Using Same

(71) Applicant: I-Mab Biopharma US Limited, Gaithersburg, MD (US)

(72) Inventors: Zhengyi Wang, Shanghai (CN); Wei Cao, Shanghai (CN); Lei Fang, Shanghai (CN); Bingshi Guo, Shanghai (CN)

(73) Assignee: I-Mab Biopharma US Limited, Gaithersburg, MD (US)

(21) Appl. No.: 17/257,294

(22) PCT Filed: Oct. 31, 2019
(86) PCT No.: PCT/CN2019/114662

§ 371 Date: Dec. 30, 2020

(30) Foreign Application Priority Data
Oct. 31, 2018 (CN) PCT/CN2018/113126

(51) Int. Cl. C07K 16/28; A61P 35/00; C07K 14/535; C12N 9/80

(52) U.S. Cl. CPC C07K 16/2803; A61P 35/00; A61K 2039/505; C12N 9/80; C07K 14/535

(57) ABSTRACT

Provided are pharmaceutical compositions each comprising a glycosylation agent, an antibody that binds to human CD47, and a pharmaceutically acceptable carrier. Also provided are novel CD47 antibodies that bind to human CD47, wherein the antibodies bind to at least one epitope that is at one of the glycosylation sites of the CD47 protein of red blood cells (e. g, at least a glycosylation site near the epitope residues Gln31 and Thr34 in CD47).

### REFERENCE TO RELATED APPLICATION

This application claims priority to International Application No. PCT/CN2018/113126, filed on Oct. 31, 2018, the contents of which are incorporated herein by reference in their entirety.

### BACKGROUND OF THE INVENTION

CD47 (Cluster of Differentiation 47), with its sequenced listed as SECT ID No: 107, was first identified as a tumor antigen on human ovarian cancer in the 1980s. Since then, CD47 has been found to be expressed on multiple human tumor types including acute myeloid leukemia (AML), chronic myeloid leukemia, acute lymphoblastic leukemia (ALL), non-Hodgkin's lymphoma (NHL), multiple myeloma (MM), bladder cancer, and other solid tumors. High levels of CD47 allow cancer cells to avoid phagocytosis despite having a higher level of calreticulin—the dominant pro-phagocytic signal.

Also known as integrin-associated protein (IAP), ovarian cancer antigen OA3, Rh-related antigen and MERG, CD47 is a multi-spanning transmembrane receptor belonging to the immunoglobulin superfamily. Its expression and activity have been implicated in a number of diseases and disorders. It is a broadly expressed transmembrane glycoprotein with a single Ig-like domain and five membrane spanning regions, which functions as a cellular ligand for SIRPα with binding mediated through the NH₂-terminal V-like domain of signal-regulatory-protein α (SIRPα). SIRPα is expressed primarily on myeloid cells, including macrophages, granulocytes, myeloid dendritic cells (DCs), mast cells, and their precursors, including hematopoietic stem cells.

Macrophages dear pathogens and damaged or aged cells from the blood stream via phagocytosis. Cell-surface CD47 interacts with its receptor on macrophages, SIRPα, to inhibit phagocytosis of normal, healthy cells. SIRPα inhibits the phagocytosis of host cells by macrophages, where the ligation of SIRPαon macrophages by CD47 expressed on the host target cell generates an inhibitory signal mediated by SHP-1 that negatively regulates phagocytosis.

In keeping with the role of CD47 to inhibit phagocytosis of normal cells, there is evidence that it is transiently up-regulated on hematopoietic stem cells (HSCs) and progenitors just prior to and during their migratory phase, and that the level of CD47 on these cells determines the probability that they are engulfed in vivo.

CD47 is also constitutively up-regulated on a number of cancers, including myeloid leukemias. Overexpression of CD47 on a myeloid leukemia line increases its pathogenicity by allowing it to evade phagocytosis. It has been concluded that CD47 up-regulation is an important mechanism for providing protection to normal HSCs during inflammation-mediated mobilization, and that leukemic progenitors co-opt this ability in order to evade macrophage killing.

Certain CD47 antibodies have been shown to restore phagocytosis and prevent atherosclerosis. See, e.g., Kojima et al., Nature, Vol. 36, 86-90 (Aug. 4, 2016). The present invention provides novel CD47 antibodies or immunologically active fragments thereof that have low immunogenicity in humans and cause low or no level of red blood cell depletion. As well known to a person skilled in the art, such antibodies may be interchangeably called “anti-CD47 antibodies.”

It is know that red blood cells have 6 N-linked glycosylation sites [(each an asparagine (Asn)] in their CD47 protein. We analyzed these 6 Asn glycosylation sites and found one of the Asp resided nearby the epitope of CD47 antibodies of the present invention.

### SUMMARY OF THE PRESENT INVENTION

In one aspect, the present invention provides isolated antibodies and their immunologically active fragments that bind to human CD47 with low or no binding to human red blood cells. For brevity, these CD47-binding isolated monoclonal antibodies and their immunologically active fragments are referred to hereinafter as “CD47 antibodies”. The CD47 antibodies of this invention are capable of modulating, e.g., blocking, inhibiting, reducing, antagonizing, neutralizing or otherwise interfering with, CD47 expression, activity and/or signaling, or the interaction between CD47 and SIRPα. Very significantly, the CD47 antibodies of this invention have low (e.g., below 10%) or no binding to red blood cells and therefore do not generally cause a significant level of depletion or hemagglutination of human red blood cells, and surprisingly in many cases do not cause any depletion or hemagglutination of human red blood cells at all. Additionally, the CD47 antibodies of this invention have exhibited potent anti-tumor activities. Unexpectedly, the CD47 antibodies of this invention (e.g., 13H3) bind to CD47 at least at the epitope residues that are nearby a glycosylation site of the red blood cells, e.g., at epitope residues Gln31 and Thr34 in CD47, which may have contributed to the low or no binding of the antibody to red blood cells.

In some embodiments, the CD47 antibodies of this invention are monoclonal antibodies each including (a) a variable heavy (VH) chain sequence that is at least 90% (e.g., at least 95%) identical to an amino acid sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, and SEQ ID NO: 75; and (b) a variable light (VL) chain sequence that is at least 90% (e.g., at least 95%) identical to an amino acid sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, and SEQ ID NO: 76, and SEQ ID NO: 78.

In some other embodiments, the CD47 antibodies of this invention each include paired VH/VL chain sequences that are at least 90% (e.g., at least 95%, 95%, 96, 97%, 98%, 99%, or 99.5%) identical to a pair of VH and VL amino acid sequences selected from the group consisting of SEQ ID NO: 1 and SEQ ID NO: 2 (i.e., 1A1), SEQ ID NO: 3 and SEQ ID NO: 4 (i.e., 1F8), SEQ ID NO: 5 and SEQ ID NO: 6 (i.e., 2A11), SEQ ID NO: 7 and SEQ ID NO: 8 (i.e., 2C2), SEQ ID NO: 9 and SEQ ID NO: 10 (i.e., 2D7), SEQ ID NO: 11 and SEQ ID NO: 12 (i.e., 2G4), SEQ ID NO: 13 and SEQ ID NO: 14 (i.e., 2G11), SEQ ID NO: 15 and SEQ ID NO: 16 (i.e., 6F4), SEQ ID NO: 17 and SEQ ID NO: 18 (i.e., 5H1), SEQ ID NO: 19 and SEQ ID NO: 20 (i.e., 5F6), SEQ ID NO: 21 and SEQ ID NO: 22 (i.e., 1F3), SEQ ID NO: 23 and SEQ ID NO: 24 (i.e., 2A4), SEQ ID NO: 25 and SEQ ID NO: 26 (i.e., 2B12), SEQ ID NO: 27 and SEQ ID NO: 28 (i.e., 13A11), SEQ ID NO: 29 and SEQ ID NO: 30 (i.e., 15E1), SEQ ID NO: 31 and SEQ ID NO: 32 (i.e., 13H3), SEQ ID NO: 33 and SEQ ID NO: 34 (i.e., 14A8), SEQ ID NO: 35 and SEQ ID NO: 36 (i.e., 16H3), SEQ ID NO: 37 and SEQ ID NO: 38 (i.e., 1A1), SEQ ID NO: 39 and SEQ ID NO: 40 (i.e., 1A1-A), SEQ ID NO: 41 and SEQ ID NO: 42 (i.e., 1A1-Q), SEQ ID NO: 43 and SEQ ID NO: 44 (i.e., 1A2), SEQ ID NO: 45 and SEQ ID NO: 46 (i.e., 1A8), SEQ ID NO: 47 and SEQ ID NO: 48 (i.e., 1B1), SEQ ID NO: 49 and SEQ ID NO: 50 (i.e., 1B2), SEQ ID NO: 51 and SEQ ID NO: 52 (i.e., 1H3), SEQ ID NO: 53 and SEQ ID NO: 54 (i.e., 1H3-Q), SEQ ID NO: 55 and SEQ ID NO: 56 (i.e., 1H3-A), SEQ ID NO: 57 and SEQ ID NO: 58 (i.e., 2A2), SEQ ID NO: 59 and SEQ ID NO: 60 (i.e., 2A3), SEQ ID NO: 61 and SEQ ID NO: 62 (i.e., 2A6), SEQ ID NO: 63 and SEQ ID NO: 64 (i.e., 2A10), SEQ ID NO: 65 and SEQ ID NO: 66 (i.e., 2B1), SEQ ID NO: 67 and SEQ ID NO: 68 (i.e., 2C6), SEQ ID NO: 69 and SEQ ID NO: 70 (i.e., 2E7), SEQ ID NO: 71 and SEQ ID NO: 72 (i.e., 2E9), SEQ ID NO: 73 and SEQ ID NO: 74 (i.e., 2F1), SEQ ID NO: 75 and SEQ ID NO: 76 (i.e., 2F3), and SEQ ID NO: 77 and SEQ ID NO: 78 (i.e., 34C5). In some instances, the CD47 antibodies of this invention each include a pair of VH and VL chain sequences selected from the group consisting of SEQ ID NO: 1 and SEQ ID NO: 2 (i.e., 1A1), SEQ ID NO: 3 and SEQ ID NO: 4 (i.e., 1F8), SEQ ID NO: 5 and SEQ ID NO: 6 (i.e., 2A11), SEQ ID NO: 7 and SEQ ID NO: 8 (i.e., 2C2), SEQ ID NO: 9 and SEQ ID NO: 10 (i.e., 2D7), SEQ ID NO: 11 and SEQ ID NO: 12 (i.e., 2G4), SEQ ID NO: 13 and SEQ ID NO: 14 (i.e., 2G11), SEQ ID NO: 15 and SEQ ID NO: 16 (i.e., 6F4), SEQ ID NO: 17 and SEQ ID NO: 18 (i.e., 5H1), SEQ ID NO: 19 and SEQ ID NO: 20 (i.e., 5F6), SEQ ID NO: 21 and SEQ ID NO: 22 (i.e., 1F3), SEQ ID NO: 23 and SEQ ID NO: 24 (i.e., 2A4), SEQ ID NO: 25 and SEQ ID NO: 26 (i.e., 2B12), SEQ ID NO: 27 and SEQ ID NO: 28 (i.e., 13A11), SEQ ID NO: 29 and SEQ ID NO: 30 (i.e., 15E1), SEQ ID NO: 31 and SEQ ID NO: 32 (i.e., 13H3), SEQ ID NO: 33 and SEQ ID NO: 34 (i.e., 14A8), SEQ ID NO: 35 and SEQ ID NO: 36 (i.e., 16H3), SEQ ID NO: 37 and SEQ ID NO: 38 (i.e., 1A1), SEQ ID NO: 39 and SEQ ID NO: 40 (i.e., 1A1-A), SEQ ID NO: 41 and SEQ ID NO: 42 (i.e., 1A1-Q), SEQ ID NO: 43 and SEQ ID NO: 44 (i.e., 1A2), SEQ ID NO: 45 and SEQ ID NO: 46 (i.e., 1A8), SEQ ID NO: 47 and SEQ ID NO: 48 (i.e., 1B1), SEQ ID NO: 49 and SEQ ID NO: 50 (i.e., 1B2), SEQ ID NO: 51 and SEQ ID NO: 52 (i.e., 1H3), SEQ ID NO: 53 and SEQ ID NO: 54 (i.e., 1H3-Q), SEQ ID NO: 55 and SEQ ID NO: 56 (i.e., 1H3-A), SEQ ID NO: 57 and SEQ ID NO: 58 (i.e., 2A2), SEQ ID NO: 59 and SEQ ID NO: 60 (i.e., 2A3), SEQ ID NO: 61 and SEQ ID NO: 62 (i.e., 2A6), SEQ ID NO: 63 and SEQ ID NO: 64 (i.e., 2A10), SEQ ID NO: 65 and SEQ ID NO: 66 (i.e., 2B1), SEQ ID NO: 67 and SEQ ID NO: 68 (i.e., 2C6), SEQ ID NO: 69 and SEQ ID NO: 70 (i.e., 2E7), SEQ ID NO: 71 and SEQ ID NO: 72 (i.e., 2E9), SEQ ID NO: 73 and SEQ ID NO: 74 (i.e., 2F1), SEQ ID NO: 75 and SEQ ID NO: 76 (i.e., 2F3), and SEQ ID NO: 77 and SEQ ID NO: 78 (i.e., 34C5).

The CD47 antibodies of this invention do not cause a significant or noticeable level of hemagglutination or depletion of red blood cells, and in many cases they do not cause hemagglutination or depletion of red blood cells at all.

In another aspect, the present invention provides isolated bispecific monoclonal antibodies. Each of such isolated bispecific monoclonal antibodies comprises a first arm and a second arm, wherein the first arm comprises a first monoclonal antibody or immunologically active fragment thereof as described above which binds human CD47, and the second arm comprise a second monoclonal antibody that does not bind human CD47. The antibody can be chimeric or humanized.

In some embodiments, the first arm of each bispecific antibodies includes (a) a variable heavy (VH) chain sequence that is at least 90% (e.g., at least 95%) identical to an amino acid sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, and SEQ ID NO: 75; and (b) a variable light (VL) chain sequence that is at least 90% (e.g., at least 95%) identical to an amino acid sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, and SEQ ID NO: 76, and SEQ ID NO: 78.

In some other embodiments, the first arm of the bispecific antibodies of this invention each includes paired VH/VL chain sequences that are at least 90% (e.g., at least 95%, 95%, 96, 97%, 98%, 99%, 99.5%, or 100%) identical to a pair of VH and VL amino acid sequences selected from the group consisting of SEQ ID NO: 1 and SEQ ID NO: 2 (i.e., 1A1), SEQ ID NO: 3 and SEQ ID NO: 4 (i.e., 1F8), SEQ ID NO: 5 and SEQ ID NO: 6 (i.e., 2A11), SEQ ID NO: 7 and SEQ ID NO: 8 (i.e., 2C2), SEQ ID NO: 9 and SEQ ID NO: 10 (i.e., 2D7), SEQ ID NO: 11 and SEQ ID NO: 12 (i.e., 2G4), SEQ ID NO: 13 and SEQ ID NO: 14 (i.e., 2G11), SEQ ID NO: 15 and SEQ ID NO: 16 (i.e., 6F4), SEQ ID NO: 17 and SEQ ID NO: 18 (i.e., 5H1), SEQ ID NO: 19 and SEQ ID NO: 20 (i.e., 5F6), SEQ ID NO: 21 and SEQ ID NO: 22 (i.e., 1F3), SEQ ID NO: 23 and SEQ ID NO: 24 (i.e., 2A4), SEQ ID NO: 25 and SEQ ID NO: 26 (i.e., 2B12), SEQ ID NO: 27 and SEQ ID NO: 28 (i.e., 13A11), SEQ ID NO: 29 and SEQ ID NO: 30 (i.e., 15E1), SEQ ID NO: 31 and SEQ ID NO: 32 (i.e., 13H3), SEQ ID NO: 33 and SEQ ID NO: 34 (i.e., 14A8), SEQ ID NO: 35 and SEQ ID NO: 36 (i.e., 16H3), SEQ ID NO: 37 and SEQ ID NO: 38 (i.e., 1A1), SEQ ID NO: 39 and SEQ ID NO: 40 (i.e., 1A1-A), SEQ ID NO: 41 and SEQ ID NO: 42 (i.e., 1A1-Q), SEQ ID NO: 43 and SEQ ID NO: 44 (i.e., 1A2), SEQ ID NO: 45 and SEQ ID NO: 46 (i.e., 1A8), SEQ ID NO: 47 and SEQ ID NO: 48 (i.e., 1B1), SEQ ID NO: 49 and SEQ ID NO: 50 (i.e., 1B2), SEQ ID NO: 51 and SEQ ID NO: 52 (i.e., 1H3), SEQ ID NO: 53 and SEQ ID NO: 54 (i.e., 1H3-Q), SEQ ID NO: 55 and SEQ ID NO: 56 (i.e., 1H3-A), SEQ ID NO: 57 and SEQ ID NO: 58 (i.e., 2A2), SEQ ID NO: 59 and SEQ ID NO: 60 (i.e., 2A3), SEQ ID NO: 61 and SEQ ID NO: 62 (i.e., 2A6), SEQ ID NO: 63 and SEQ ID NO: 64 (i.e., 2A10), SEQ ID NO: 65 and SEQ ID NO: 66 (i.e., 2B1), SEQ ID NO: 67 and SEQ ID NO: 68 (i.e., 2C6), SEQ ID NO: 69 and SEQ ID NO: 70 (i.e., 2E7), SEQ ID NO: 71 and SEQ ID NO: 72 (i.e., 2E9), SEQ ID NO: 73 and SEQ ID NO: 74 (i.e., 2F1), SEQ ID NO: 75 and SEQ ID NO: 76 (i.e., 2F3), and SEQ ID NO: 77 and SEQ ID NO: 78 (i.e., 34C5). In some instances, the first arm of each bispecific CD47 antibody of this invention each include a pair of VH and VL chain sequences selected from the group consisting of SEQ ID NO: 1 and SEQ ID NO: 2 (i.e., 1A1), SEQ ID NO: 3 and SEQ ID NO: 4 (i.e., 1F8), SEQ ID NO: 5 and SEQ ID NO: 6 (i.e., 2A11), SEQ ID NO: 7 and SEQ ID NO: 8 (i.e., 2C2), SEQ ID NO: 9 and SEQ ID NO: 10 (i.e., 2D7), SEQ ID NO: 11 and SEQ ID NO: 12 (i.e., 2G4), SEQ ID NO: 13 and SEQ ID NO: 14 (i.e., 2G11), SEQ ID NO: 15 and SEQ ID NO: 16 (i.e., 6F4), SEQ ID NO: 17 and SEQ ID NO: 18 (i.e., 5H1), SEQ ID NO: 19 and SEQ ID NO: 20 (i.e., 5F6), SEQ ID NO: 21 and SEQ ID NO: 22 (i.e., 1F3), SEQ ID NO: 23 and SEQ ID NO: 24 (i.e., 2A4), SEQ ID NO: 25 and SEQ ID NO: 26 (i.e., 2B12), SEQ ID NO: 27 and SEQ ID NO: 28 (i.e., 13A11), SEQ ID NO: 29 and SEQ ID NO: 30 (i.e., 15E1), SEQ ID NO: 31 and SEQ ID NO: 32 (i.e., 13H3), SEQ ID NO: 33 and SEQ ID NO: 34 (i.e., 14A8), SEQ ID NO: 35 and SEQ ID NO: 36 (i.e., 16H3), SEQ ID NO: 37 and SEQ ID NO: 38 (i.e., 1A1), SEQ ID NO: 39 and SEQ ID NO: 40 (i.e., 1A1-A), SEQ ID NO: 41 and SEQ ID NO: 42 (i.e., 1A1-Q), SEQ ID NO: 43 and SEQ ID NO: 44 (i.e., 1A2), SEQ ID NO: 45 and SEQ ID NO: 46 (i.e., 1A8), SEQ ID NO: 47 and SEQ ID NO: 48 (i.e., 1B1), SEQ ID NO: 49 and SEQ ID NO: 50 (i.e., 1B2), SEQ ID NO: 51 and SEQ ID NO: 52 (i.e., 1H3), SEQ ID NO: 53 and SEQ ID NO: 54 (i.e., 1H3-Q), SEQ ID NO: 55 and SEQ ID NO: 56 (i.e., 1H3-A), SEQ ID NO: 57 and SEQ ID NO: 58 (i.e., 2A2), SEQ ID NO: 59 and SEQ ID NO: 60 (i.e., 2A3), SEQ ID NO: 61 and SEQ ID NO: 62 (i.e., 2A6), SEQ ID NO: 63 and SEQ ID NO: 64 (i.e., 2A10), SEQ ID NO: 65 and SEQ ID NO: 66 (i.e., 2B1), SEQ ID NO: 67 and SEQ ID NO: 68 (i.e., 2C6), SEQ ID NO: 69 and SEQ ID NO: 70 (i.e., 2E7), SEQ ID NO: 71 and SEQ ID NO: 72 (i.e., 2E9), SEQ ID NO: 73 and SEQ ID NO: 74 (i.e., 2F1), SEQ ID NO: 75 and SEQ ID NO: 76 (i.e., 2F3), and SEQ ID NO: 77 and SEQ ID NO: 78 (i.e., 34C5).

In some embodiments, the second arm in the isolated bispecific monoclonal antibodies binds to a cancer cell.

In some other embodiments, the bispecific monoclonal antibodies inhibit interaction between human CD47 and human SIRPα.

In another aspect, the present invention provides pharmaceutical compositions each including at least a glycosylation agent, an antibody that binds to human CD47, and a pharmaceutically acceptable carrier.

In some embodiments, the antibody contained in the pharmaceutical compositions of this invention is a monoclonal antibody, a bispecific antibody, or a fusion protein comprising a monoclonal or bispecific antibody and a cytokine.

A suitable monoclonal antibody may include (1) a variable heavy (VH) chain sequence that is at least 95% identical to an amino acid sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, and SEQ ID NO: 77; and (2) a variable light (VL) chain sequence that is at least 95% identical to an amino acid sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, and SEQ ID NO: 78.

For example, each monoclonal antibody contained in the pharmaceutical compositions of this invention includes a VH/VL pair and the VH/VL pair comprises VH and VL chain sequences at least 95% identical to a pair of VH and VL amino acid sequences selected from the group consisting of SEQ ID NO: 1 and SEQ ID NO: 2, SEQ ID NO: 3 and SEQ ID NO: 4, SEQ ID NO: 5 and SEQ ID NO: 6, SEQ ID NO: 7 and SEQ ID NO: 8, SEQ ID NO: 9 and SEQ ID NO: 10, SEQ ID NO: 11 and SEQ ID NO: 12, SEQ ID NO: 13 and SEQ ID NO: 14, SEQ ID NO: 15 and SEQ ID NO: 16, SEQ ID NO: 17 and SEQ ID NO: 18, SEQ ID NO: 19 and SEQ ID NO: 20, SEQ ID NO: 21 and SEQ ID NO: 22, SEQ ID NO: 23 and SEQ ID NO: 24, SEQ ID NO: 25 and SEQ ID NO: 26, SEQ ID NO: 27 and SEQ ID NO: 28, SEQ ID NO: 29 and SEQ ID NO: 30, SEQ ID NO: 31 and SEQ ID NO: 32, SEQ ID NO: 33 and SEQ ID NO: 34, SEQ ID NO: 35 and SEQ ID NO: 36, SEQ ID NO: 37 and SEQ ID NO: 38, SEQ ID NO: 39 and SEQ ID NO: 40, SEQ ID NO: 41 and SEQ ID NO: 42, SEQ ID NO: 43 and SEQ ID NO: 44, SEQ ID NO: 45 and SEQ ID NO: 46, SEQ ID NO: 47 and SEQ ID NO: 48, SEQ ID NO: 49 and SEQ ID NO: 50, SEQ ID NO: 51 and SEQ ID NO: 52, SEQ ID NO: 53 and SEQ ID NO: 54, SEQ ID NO: 55 and SEQ ID NO: 56, SEQ ID NO: 57 and SEQ ID NO: 58, SEQ ID NO: 59 and SEQ ID NO: 60, SEQ ID NO: 61 and SEQ ID NO: 62, SEQ ID NO: 63 and SEQ ID NO: 64, SEQ ID NO: 65 and SEQ ID NO: 66, SEQ ID NO: 67 and SEQ ID NO: 68, SEQ ID NO: 69 and SEQ ID NO: 70, SEQ ID NO: 71 and SEQ ID NO: 72, SEQ ID NO: 73 and SEQ ID NO: 74, SEQ ID NO: 75 and SEQ ID NO: 76, and SEQ ID NO: 77 and SEQ ID NO: 78.

In some other embodiments, the VH/VL pair includes VH and VL chain sequences selected from the group consisting of SEQ ID NO: 1 and SEQ ID NO: 2, SEQ ID NO: 3 and SEQ ID NO: 4, SEQ ID NO: 5 and SEQ ID NO: 6, SEQ ID NO: 7 and SEQ ID NO: 8, SEQ ID NO: 9 and SEQ ID NO: 10, SEQ ID NO: 11 and SEQ ID NO: 12, SEQ ID NO: 13 and SEQ ID NO: 14, SEQ ID NO: 15 and SEQ ID NO: 16, SEQ ID NO: 17 and SEQ ID NO: 18, SEQ ID NO: 19 and SEQ ID NO: 20, SEQ ID NO: 21 and SEQ ID NO: 22, SEQ ID NO: 23 and SEQ ID NO: 24, SEQ ID NO: 25 and SEQ ID NO: 26, SEQ ID NO: 27 and SEQ ID NO: 28, SEQ ID NO: 29 and SEQ ID NO: 30, SEQ ID NO: 31 and SEQ ID NO: 32, SEQ ID NO: 33 and SEQ ID NO: 34, SEQ ID NO: 35 and SEQ ID NO: 36, SEQ ID NO: 37 and SEQ ID NO: 38, SEQ ID NO: 39 and SEQ ID NO: 40, SEQ ID NO: 41 and SEQ ID NO: 42, SEQ ID NO: 43 and SEQ ID NO: 44, SEQ ID NO: 45 and SEQ ID NO: 46, SEQ ID NO: 47 and SEQ ID NO: 48, SEQ ID NO: 49 and SEQ ID NO: 50, SEQ ID NO: 51 and SEQ ID NO: 52, SEQ ID NO: 53 and SEQ ID NO: 54, SEQ ID NO: 55 and SEQ ID NO: 56, SEQ ID NO: 57 and SEQ ID NO: 58, SEQ ID NO: 59 and SEQ ID NO: 60, SEQ ID NO: 61 and SEQ ID NO: 62, SEQ ID NO: 63 and SEQ ID NO: 64, SEQ ID NO: 65 and SEQ ID NO: 66, SEQ ID NO: 67 and SEQ ID NO: 68, SEQ ID NO: 69 and SEQ ID NO: 70, SEQ ID NO: 71 and SEQ ID NO: 72, SEQ ID NO: 73 and SEQ ID NO: 74, SEQ ID NO: 75 and SEQ ID NO: 76, and SEQ ID NO: 77 and SEQ ID NO: 78.

A bispecific antibody contained in the pharmaceutical compositions of this invention includes a first arm and a second arm, the first arm comprises a first monoclonal antibody which binds human CD47, and the second arm comprise a second monoclonal antibody that does not bind human CD47. The first arm may be any monoclonal antibody immediately described above. As to the second arm in the bispecific antibody, it can bind to a cancer cell.

Also in some embodiments, the monoclonal or bispecific antibodies contained in the pharmaceutical compositions of this invention may exist in the form of a fusion protein that includes the monoclonal or bispecific antibody and a cytokine, with or without a linked between the monoclonal or bispecific antibody and the cytokine. The cytokine can be a wild type or a variant of an immunoglobulin (Ig), a hemopoietic growth factor, an interferon, a tumor necrosis factor, an interleukin-17 receptor, or a monomeric glycoprotein.

In some embodiments, the cytokine contained in the fusion proteins is a wild type or a variant of the monomeric glycoprotein. In some other embodiments, the cytokine is a wild type or variant of granulocyte-macrophage colony-stimulating factor (GM-CSF). In yet some other embodiments, the antibody is fused to the cytokine without a linker, or with a linker selected from the group consisting of (G4S)3, (G4S)6, (GS)9, IGD(F30), IGD(F64), IGD(R30), IGN(R64), IGD(R30-Cys), and IGD(R64-Cys).

In some embodiments of the compositions of this invention, the glycosylating agent comprises glycosyl group connected or bonded to a leaving group which is a halo, thioalkyl, imidate, acetate, phosphate, or O-pentenyl group.

Another aspect of this invention provides a method for increasing the selective of binding of a CD47 antibody to human CD47 while decreasing the binding of the CD47 antibody to red blood cells, comprising treating the red blood cells with a glycosylation agent before subjecting the human CD47 to the CD47 antibody.

In some embodiments, the method further includes subjecting the human CD47 with the CD47 antibody.

In some embodiments, the method further includes deglycosylation of the red blood cells. For example, the deglysosylation step can be carried out with a PNGase (i.e., Peptide:N-glycosidase), e.g., Peptide:N-glycosidase F (commonly referred to as PNGase F) which is an amidase of the peptide-N4-(N-acetyl-beta-glucosaminyl)asparagine amidase class.

In some embodiments, the method further includes subjecting the human CD47 with the CD47 antibody.

In some embodiments, the CD47 antibody is an isolated monoclonal antibody or immunologically active fragment thereof that binds to human CD47 and the CD antibody comprises:

a variable heavy (VH) chain sequence that is at least 95% identical to an amino acid sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, and SEQ ID NO: 77; and

a variable light (VL) chain sequence that is at least 95% identical to an amino acid sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, and SEQ ID NO: 78.

In some embodiments of the method of this invention, the CD47 antibody includes a VH/VL pair, wherein the VH/VL pair comprises VH and VL chain sequences at least 95% identical to a pair of VH and VL amino acid sequences selected from the group consisting of SEQ ID NO: 1 and SEQ ID NO: 2, SEQ ID NO: 3 and SEQ ID NO: 4, SEQ ID NO: 5 and SEQ ID NO: 6, SEQ ID NO: 7 and SEQ ID NO: 8, SEQ ID NO: 9 and SEQ ID NO: 10, SEQ ID NO: 11 and SEQ ID NO: 12, SEQ ID NO: 13 and SEQ ID NO: 14, SEQ ID NO: 15 and SEQ ID NO: 16, SEQ ID NO: 17 and SEQ ID NO: 18, SEQ ID NO: 19 and SEQ ID NO: 20, SEQ ID NO: 21 and SEQ ID NO: 22, SEQ ID NO: 23 and SEQ ID NO: 24, SEQ ID NO: 25 and SEQ ID NO: 26, SEQ ID NO: 27 and SEQ ID NO: 28, SEQ ID NO: 29 and SEQ ID NO: 30, SEQ ID NO: 31 and SEQ ID NO: 32, SEQ ID NO: 33 and SEQ ID NO: 34, SEQ ID NO: 35 and SEQ ID NO: 36, SEQ ID NO: 37 and SEQ ID NO: 38, SEQ ID NO: 39 and SEQ ID NO: 40, SEQ ID NO: 41 and SEQ ID NO: 42, SEQ ID NO: 43 and SEQ ID NO: 44, SEQ ID NO: 45 and SEQ ID NO: 46, SEQ ID NO: 47 and SEQ ID NO: 48, SEQ ID NO: 49 and SEQ ID NO: 50, SEQ ID NO: 51 and SEQ ID NO: 52, SEQ ID NO: 53 and SEQ ID NO: 54, SEQ ID NO: 55 and SEQ ID NO: 56, SEQ ID NO: 57 and SEQ ID NO: 58, SEQ ID NO: 59 and SEQ ID NO: 60, SEQ ID NO: 61 and SEQ ID NO: 62, SEQ ID NO: 63 and SEQ ID NO: 64, SEQ ID NO: 65 and SEQ ID NO: 66, SEQ ID NO: 67 and SEQ ID NO: 68, SEQ ID NO: 69 and SEQ ID NO: 70, SEQ ID NO: 71 and SEQ ID NO: 72, SEQ ID NO: 73 and SEQ ID NO: 74, SEQ ID NO: 75 and SEQ ID NO: 76, and SEQ ID NO: 77 and SEQ ID NO: 78.

In some embodiments of the method of this invention, the CD47 antibody comprises a VH/VL pair, wherein the VH/VL pair comprises VH and VL chain sequences selected from the group consisting of SEQ ID NO: 1 and SEQ ID NO: 2, SEQ ID NO: 3 and SEQ ID NO: 4, SEQ ID NO: 5 and SEQ ID NO: 6, SEQ ID NO: 7 and SEQ ID NO: 8, SEQ ID NO: 9 and SEQ ID NO: 10, SEQ ID NO: 11 and SEQ ID NO: 12, SEQ ID NO: 13 and SEQ ID NO: 14, SEQ ID NO: 15 and SEQ ID NO: 16, SEQ ID NO: 17 and SEQ ID NO: 18, SEQ ID NO: 19 and SEQ ID NO: 20, SEQ ID NO: 21 and SEQ ID NO: 22, SEQ ID NO: 23 and SEQ ID NO: 24, SEQ ID NO: 25 and SEQ ID NO: 26, SEQ ID NO: 27 and SEQ ID NO: 28, SEQ ID NO: 29 and SEQ ID NO: 30, SEQ ID NO: 31 and SEQ ID NO: 32, SEQ ID NO: 33 and SEQ ID NO: 34, SEQ ID NO: 35 and SEQ ID NO: 36, SEQ ID NO: 37 and SEQ ID NO: 38, SEQ ID NO: 39 and SEQ ID NO: 40, SEQ ID NO: 41 and SEQ ID NO: 42, SEQ ID NO: 43 and SEQ ID NO: 44, SEQ ID NO: 45 and SEQ ID NO: 46, SEQ ID NO: 47 and SEQ ID NO: 48, SEQ ID NO: 49 and SEQ ID NO: 50, SEQ ID NO: 51 and SEQ ID NO: 52, SEQ ID NO: 53 and SEQ ID NO: 54, SEQ ID NO: 55 and SEQ ID NO: 56, SEQ ID NO: 57 and SEQ ID NO: 58, SEQ ID NO: 59 and SEQ ID NO: 60, SEQ ID NO: 61 and SEQ ID NO: 62, SEQ ID NO: 63 and SEQ ID NO: 64, SEQ ID NO: 65 and SEQ ID NO: 66, SEQ ID NO: 67 and SEQ ID NO: 68, SEQ ID NO: 69 and SEQ ID NO: 70, SEQ ID NO: 71 and SEQ ID NO: 72, SEQ ID NO: 73 and SEQ ID NO: 74, SEQ ID NO: 75 and SEQ ID NO: 76, and SEQ ID NO: 77 and SEQ ID NO: 78.

In still other embodiments of the method of this invention, the CD47 antibody is chimeric or humanized.

In still other embodiments of the method of this invention, the CD47 antibody prevents human CD47 from interacting with signal-regulatory-protein α (SIRPα).

In still other embodiments of the method of this invention, the CD47 antibody promotes macrophage-mediated phagocytosis of a CD47-expressing cell.

In still other embodiments of the method of this invention, the CD47 antibody does not cause a significant level of hemagglutination or depletion of red blood cells.

In still other embodiments of the method of this invention, the CD47 antibody does not cause hemagglutination or depletion of red blood cells.

In still other embodiments of the method of this invention, the CD47 antibody is bispecific and comprises a first arm and a second arm, wherein the first arm comprises a first monoclonal antibody or immunologically active fragment thereof as described above and binds human CD47, and the second arm comprise a second monoclonal antibody that does not bind human CD47. The second arm can bind to a cancer cell.

In still other embodiments of the method of this invention, the bispecific antibody inhibits interaction between human CD47 and human SIRPα.

Another aspect of this invention provides a method for binding a CD47 antibody to human CD47 without significantly binding the CD47 antibody to red blood cells, comprising glycosylating the red blood cells with a glycosylation agent.

In some embodiments, the red blood cells are glycosylated at one or more N-linked glycosylation sites (Asn).

In some other embodiments, Asn32 located nearby the critical epitope residue Gln31 and Thr34 is glysosylated.

As used herein, the term “pharmaceutically acceptable carrier or excipient” refers to a carrier or an excipient that is useful for preparing a pharmaceutical composition or formulation that is generally safe, non-toxic, and neither biologically nor otherwise undesirable. A carrier or excipient employed is typically one suitable for administration to human subjects or other mammals. In making the compositions, the active ingredient is usually mixed with, diluted by, or enclosed with a carrier or excipient. When the carrier or excipient serves as a diluent, it can be a solid, semi-solid, or liquid material, which acts as a vehicle, carrier, or medium for the active ingredient of the antibody.

Also within the scope of the present invention is a method for treating a disease in a human subject in need thereof, and the method includes administering to the subject a therapeutically effective amount of a CD47 antibody of this invention, a bispecific monoclonal antibody of this invention, or a pharmaceutical composition of this invention, and the disease is a cancer, a fibrotic disease, or any disease related to inhibition of phagocytosis. In some instance, the cancer can be selected from the group consisting of ovarian cancer, colon cancer, breast cancer, lung cancer, head and neck cancer, bladder cancer, colorectal cancer, pancreatic cancer, non-Hodgkin's lymphoma, acute lymphocytic leukemia, chronic lymphocytic leukemia, acute myeloid leukemia, chronic myelogenous leukemia, hairy cell leukemia (HCL), T-cell prolymphocytic leukemia (T-PLL), large granular lymphocytic leukemia, adult T-cell leukemia, multiple myeloma, melanoma, leiomyoma, leiomyosarcoma, glioma, glioblastoma, myelomas, monocytic leukemias, B-cell derived leukemias, T-cell derived leukemias, B-cell derived lymphomas, T-cell derived lymphomas, endometrial cancer, kidney cancer, melanoma, prostate cancer, thyroid cancer, cervical cancer, gastric cancer, liver cancer, and solid tumors; whereas the fibrotic disease can be selected from the group consisting of myocardial infarction, angina, osteoarthritis, pulmonary fibrosis, asthma, cystic fibrosis, bronchitis, and asthma. Examples of solid tumors include, e.g., endometrial cancer, thyroid cancer, cervical cancer, gastric cancer, breast tumors, ovarian tumors, lung tumors, pancreatic tumors, prostate tumors, melanoma tumors, colorectal tumors, lung tumors, head and neck tumors, bladder tumors, esophageal tumors, liver tumors, and kidney tumors, and neuroblastic-derived CNS tumors. The disease related to inhibition of phagocytosis can be a cardiovascular disease (e.g., atherosclerosis, stroke, hypertensive heart disease, rheumatic heart disease, cardiomyopathy, heart arrhythmia, congenital heart disease, valvular heart disease, carditis, aortic aneurysms, peripheral artery disease, or venous thrombosis).

As used herein, the term “effective amount” refers to that amount of a CD47 antibody sufficient or required to effect treatment, prognosis or diagnosis of a disease associated with CD47 dependent signaling, as described herein, when administered to a subject. Therapeutically effective amounts of antibodies provided herein, when used alone or in combination, will vary depending upon the relative activity of the antibodies (e.g., promoting macrophage mediated phagocytosis of cancer cells expressing CD47) and depending upon the subject and disease condition being treated, the weight and age of the subject, the severity of the disease condition, the manner of administration and the like, which can readily be determined by one of ordinary skill in the art.

As used herein, the term “isolated” preceding a CD47 antibody of this invention means that the antibody is substantially free of other cellular material. In one embodiment, an isolated antibody is substantially free of other proteins from the same species. In another embodiment, an isolated antibody is expressed by a cell from a different species and is substantially free of other proteins from the different species. A protein may be rendered substantially free of naturally associated components (or components associated with the cellular expression system used to produce the antibody) by isolation, using protein purification techniques well known in the art. In one embodiment, the antibodies, or antigen binding fragments, of the invention are isolated.

Still within the scope of this invention are fusion proteins each comprising a first amino acid sequence and a second amino acid sequence, wherein the first amino acid sequence is SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, or SEQ ID NO: 77, or an amino acid sequence that is at least 90% (e.g., at least 95%) identical thereto; and the second amino acid sequence is SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, or SEQ ID NO: 78, or an amino acid sequence that is at least 90% (e.g., at least 95%) identical thereto.

In some embodiments, a fusion protein of this invention includes a combination of the first amino acid sequence and the second amino acid sequence, and the combination of these two amino acid sequences is SEQ ID NO: 1 and SEQ ID NO: 2 (i.e., 1A1), SEQ ID NO: 3 and SEQ ID NO: 4 (i.e., 1F8), SEQ ID NO: 5 and SEQ ID NO: 6 (i.e., 2A11), SEQ ID NO: 7 and SEQ ID NO: 8 (i.e., 2C2), SEQ ID NO: 9 and SEQ ID NO: 10 (i.e., 2D7), SEQ ID NO: 11 and SEQ ID NO: 12 (i.e., 2G4), SEQ ID NO: 13 and SEQ ID NO: 14 (i.e., 2G11), SEQ ID NO: 15 and SEQ ID NO: 16 (i.e., 6F4), SEQ ID NO: 17 and SEQ ID NO: 18 (i.e., 5H1), SEQ ID NO: 19 and SEQ ID NO: 20 (i.e., 5F6), SEQ ID NO: 21 and SEQ ID NO: 22 (i.e., 1F3), SEQ ID NO: 23 and SEQ ID NO: 24 (i.e., 2A4), SEQ ID NO: 25 and SEQ ID NO: 26 (i.e., 2B12), SEQ ID NO: 27 and SEQ ID NO: 28 (i.e., 13A11), SEQ ID NO: 29 and SEQ ID NO: 30 (i.e., 15E1), SEQ ID NO: 31 and SEQ ID NO: 32 (i.e., 13H3), SEQ ID NO: 33 and SEQ ID NO: 34 (i.e., 14A8), SEQ ID NO: 35 and SEQ ID NO: 36 (i.e., 16H3), SEQ ID NO: 37 and SEQ ID NO: 38 (i.e., 1A1), SEQ ID NO: 39 and SEQ ID NO: 40 (i.e., 1A1-A), SEQ ID NO: 41 and SEQ ID NO: 42 (i.e., 1A1-Q), SEQ ID NO: 43 and SEQ ID NO: 44 (i.e., 1A2), SEQ ID NO: 45 and SEQ ID NO: 46 (i.e., 1A8), SEQ ID NO: 47 and SEQ ID NO: 48 (i.e., 1B1), SEQ ID NO: 49 and SEQ ID NO: 50 (i.e., 1B2), SEQ ID NO: 51 and SEQ ID NO: 52 (i.e., 1H3), SEQ ID NO: 53 and SEQ ID NO: 54 (i.e., 1H3-Q), SEQ ID NO: 55 and SEQ ID NO: 56 (i.e., 1H3-A), SEQ ID NO: 57 and SEQ ID NO: 58 (i.e., 2A2), SEQ ID NO: 59 and SEQ ID NO: 60 (i.e., 2A3), SEQ ID NO: 61 and SEQ ID NO: 62 (i.e., 2A6), SEQ ID NO: 63 and SEQ ID NO: 64 (i.e., 2A10), SEQ ID NO: 65 and SEQ ID NO: 66 (i.e., 2B1), SEQ ID NO: 67 and SEQ ID NO: 68 (i.e., 2C6), SEQ ID NO: 69 and SEQ ID NO: 70 (i.e., 2E7), SEQ ID NO: 71 and SEQ ID NO: 72 (i.e., 2E9), SEQ ID NO: 73 and SEQ ID NO: 74 (i.e., 2F1), SEQ ID NO: 75 and SEQ ID NO: 76 (i.e., 2F3), SEQ ID NO: 77 and SEQ ID NO: 78 (i.e., 34C5), or a combination that is at least 90% (e.g., at least 95%) identical thereto for each of the first and second amino acid sequences.

In some other embodiments, a fusion protein of this invention includes a combination of the first amino acid sequence and the second amino acid sequence, and the combination of these two amino acid sequences is SEQ ID NO: 1 and SEQ ID NO: 2 (i.e., 1A1), SEQ ID NO: 3 and SEQ ID NO: 4 (i.e., 1F8), SEQ ID NO: 5 and SEQ ID NO: 6 (i.e., 2A11), SEQ ID NO: 7 and SEQ ID NO: 8 (i.e., 2C2), SEQ ID NO: 9 and SEQ ID NO: 10 (i.e., 2D7), SEQ ID NO: 11 and SEQ ID NO: 12 (i.e., 2G4), SEQ ID NO: 13 and SEQ ID NO: 14 (i.e., 2G11), SEQ ID NO: 15 and SEQ ID NO: 16 (i.e., 6F4), SEQ ID NO: 17 and SEQ ID NO: 18 (i.e., 5H1), SEQ ID NO: 19 and SEQ ID NO: 20 (i.e., 5F6), SEQ ID NO: 21 and SEQ ID NO: 22 (i.e., 1F3), SEQ ID NO: 23 and SEQ ID NO: 24 (i.e., 2A4), SEQ ID NO: 25 and SEQ ID NO: 26 (i.e., 2B12), SEQ ID NO: 27 and SEQ ID NO: 28 (i.e., 13A11), SEQ ID NO: 29 and SEQ ID NO: 30 (i.e., 15E1), SEQ ID NO: 31 and SEQ ID NO: 32 (i.e., 13H3), SEQ ID NO: 33 and SEQ ID NO: 34 (i.e., 14A8), SEQ ID NO: 35 and SEQ ID NO: 36 (i.e., 16H3), SEQ ID NO: 37 and SEQ ID NO: 38 (i.e., 1A1), SEQ ID NO: 39 and SEQ ID NO: 40 (i.e., 1A1-A), SEQ ID NO: 41 and SEQ ID NO: 42 (i.e., 1A1-Q), SEQ ID NO: 43 and SEQ ID NO: 44 (i.e., 1A2), SEQ ID NO: 45 and SEQ ID NO: 46 (i.e., 1A8), SEQ ID NO: 47 and SEQ ID NO: 48 (i.e., 1B1), SEQ ID NO: 49 and SEQ ID NO: 50 (i.e., 1B2), SEQ ID NO: 51 and SEQ ID NO: 52 (i.e., 1H3), SEQ ID NO: 53 and SEQ ID NO: 54 (i.e., 1H3-Q), SEQ ID NO: 55 and SEQ ID NO: 56 (i.e., 1H3-A), SEQ ID NO: 57 and SEQ ID NO: 58 (i.e., 2A2), SEQ ID NO: 59 and SEQ ID NO: 60 (i.e., 2A3), SEQ ID NO: 61 and SEQ ID NO: 62 (i.e., 2A6), SEQ ID NO: 63 and SEQ ID NO: 64 (i.e., 2A10), SEQ ID NO: 65 and SEQ ID NO: 66 (i.e., 2B1), SEQ ID NO: 67 and SEQ ID NO: 68 (i.e., 2C6), SEQ ID NO: 69 and SEQ ID NO: 70 (i.e., 2E7), SEQ ID NO: 71 and SEQ ID NO: 72 (i.e., 2E9), SEQ ID NO: 73 and SEQ ID NO: 74 (i.e., 2F1), SEQ ID NO: 75 and SEQ ID NO: 76 (i.e., 2F3), or SEQ ID NO: 77 and SEQ ID NO: 78 (i.e., 34C5).

In still some other embodiments, a fusion protein of this invention can further include an additional protein—in addition to the first and second amino acid sequences. The additional protein is an antibody or a cytokine.

In yet still some other embodiments, a fusion protein of this invention can be conjugated with a small-molecule therapeutic agent (e.g., anti-cancer or anti-inflammation agent) or a marker (e.g., a biomarker or fluorescent marker).

In yet another aspect, the present invention provides immunodominant epitopes encoded by the CD47 gene comprising a recombinant protein containing conformationally a TNMEAQ loop (residues 26-31), T34, E35, L74, and an LTR hinge (residues 101-103) of CD47.

In yet still another aspect, the present invention provides biological molecules that specifically bind to a conformational epitope having an amino acid sequence comprising a TNMEAQ loop (residues 26-31), T34, E35, L74, and an LTR hinge (residues 101-103) of CD47, wherein the antibody can specifically bind to CD47.

As used herein, the term “biological molecules” is meant to include synthetic antibodies (monoclonal or bispecific), peptides, and biomimetic molecules. The term “biomimetic molecules” refers to molecules which are designed or developed to have structures or properties similar to or resembling those of naturally occurring large compounds such as proteins or nucleotides and which have a molecular weight of, e.g., at least 3,000, at least 5,000, or at least 10,000.

All references cited herein are incorporated by reference in their entirety.

### BRIEF DESCRIPTIONS OF THE DRAWINGS

FIG. 1 shows dose-dependent response of CD47 antibodies binding to monomeric CD47-ECD.

FIG. 2a and FIG. 2b show dose-dependent response of CD47 antibodies binding to dimeric CD47-ECD.

FIG. 3a, FIG. 3b, and FIG. 3c dose-dependent response of CD47 antibodies blocking the binding of CD47 to SIRPα.

FIG. 4a and FIG. 4b show dose-dependent response of CD47 antibodies binding to CD47+ Raji cells; and FIG. 4c, FIG. 4d and FIG. 4e show binding kinetics and data of CD47 antibodies as measured by Biocore analysis.

FIG. 5a and FIG. 5b show phagocytosis of tumor cells by human MΦ with CD47 antibodies.

FIGS. 6a-6c show macrophage-mediated phagocytosis of various human blood cancer cell lines triggered by CD47 antibodies.

FIGS. 7a and 7b show red blood cells (RBC)-sparing properties in RBC agglutination assay with CD47 antibodies.

FIGS. 8a, 8b, 8c, and 8d show activities to bind RBC and induce RBC agglutination by CD antibodies at different and higher doses.

FIGS. 9a, 9b, 9c, and 9d show RBC-binding activities of CD47 antibodies.

FIG. 10 shows results of red blood cell agglutination across multiple human blood samples induced by CD47 antibodies.

FIG. 11 shows the human platelet binding activities of CD47 antibodies and SIRPa-Ig fusion, with CD61 stained as a surface marker for platelets.

FIG. 12 shows the test results of cyno red blood cell agglutination induced by CD47 antibodies and SIRPa-Ig fusion in vitro.

FIG. 13 shows the test results of phagocytosis and AML cells binding by CD47 antibodies and control.

FIG. 14a and FIG. 14b show the efficacy of treatments with CD47 antibodies and control on luciferase-Raji xenograft mice.

FIG. 15 shows the polarization of macrophage in tumor-bearing mice induced by CD47 antibodies and control.

FIG. 16 shows the CD47 expression profiles using PDX samples of various human cancer types.

FIG. 17 shows results of safety pharm study (hematology) in cynomolgus monkeys.

FIG. 18 shows completion in binding of CD47 between antibodies 1F8 and 5F9, and between antibodies 1F8 and 2A1, due to their different epitopes, and structures of the 5F9/CD47 complex and the 1F8/CD47 complex.

FIGS. 19a, 19b, 19c, 19d, 19e, 19f, 19g, and 19h show the effects of the CD47 antibody 13H3 on RBC congregation, hemoglobin, platelets, and lymphocytes, respectively.

FIG. 20 shows strong binding affinity of 34C5 to recombinant CD47-ECD.

FIG. 21 shows strong binding affinity of 34C5 to CD47-bearing Raji cells.

FIG. 22 shows that 34C5 was able to effectively block CD47 binding to SIRPα, with an EC₅₀ of 0.30 nM.

FIG. 23 shows that the antibody 34C5 promoted phagocystosis of tumor cells by human MCP.

FIG. 24 shows the antibody 34C5 did not cause in vitro RBC agglutination.

FIG. 25 shows the antibody 34C5 decrease its binding to RBC with the decreasing concentration of this antibody.

FIG. 26 shows amino acid sequences of some CD47 antibodies of this invention and their respective nucleotide sequences.

FIG. 27 shows the amino acid sequence for CD47 Immunoglobulin-like domain (Ig-V) 19-141.

FIGS. 28A and 28B show binding activities of CD47 antibodies after deglycosylation of RBC.

### DETAILED DESCRIPTION OF THE INVENTION

The present invention provides novel isolated monoclonal CD47 antibodies that can prevent human CD47 from interacting with SIRPα, or promote macrophage-mediated phagocytosis of a CD47-expressing cell. These CD47 antibodies do not cause a significant or noticeable level of hemagglutination or depletion of red blood cells, and in many cases they do not cause hemagglutination or depletion of red blood cells at all.

As examples, a CD47 antibodies of this invention would include (a) a variable heavy (VH) chain sequence that is at least 90% (e.g., at least 95%) identical to an amino acid sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, and SEQ ID NO: 77; and (b) a variable light (VL) chain sequence that is at least 90% (e.g., at least 95%) identical to an amino acid sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, and SEQ ID NO: 78. In some further instance, a CD47 antibodies of this invention would include a combined VH/VL chain sequence that is at least 90% (e.g., at least 95%) identical to an amino acid sequence selected from the group consisting of SEQ ID NO: 1 and SEQ ID NO: 2, SEQ ID NO: 3 and SEQ ID NO: 4, SEQ ID NO: 5 and SEQ ID NO: 6, SEQ ID NO: 7 and SEQ ID NO: 8, SEQ ID NO: 9 and SEQ ID NO: 10, SEQ ID NO: 11 and SEQ ID NO: 12, SEQ ID NO: 13 and SEQ ID NO: 14, SEQ ID NO: 15 and SEQ ID NO: 16, SEQ ID NO: 17 and SEQ ID NO: 18, SEQ ID NO: 19 and SEQ ID NO: 20, SEQ ID NO: 21 and SEQ ID NO: 22, SEQ ID NO: 23 and SEQ ID NO: 24, SEQ ID NO: 25 and SEQ ID NO: 26, SEQ ID NO: 27 and SEQ ID NO: 28, SEQ ID NO: 29 and SEQ ID NO: 30, SEQ ID NO: 31 and SEQ ID NO: 32, SEQ ID NO: 33 and SEQ ID NO: 34, SEQ ID NO: 35 and SEQ ID NO: 36, SEQ ID NO: 37 and SEQ ID NO: 38, SEQ ID NO: 39 and SEQ ID NO: 40, SEQ ID NO: 41 and SEQ ID NO: 42, SEQ ID NO: 43 and SEQ ID NO: 44, SEQ ID NO: 45 and SEQ ID NO: 46, SEQ ID NO: 47 and SEQ ID NO: 48, SEQ ID NO: 49 and SEQ ID NO: 50, SEQ ID NO: 51 and SEQ ID NO: 52, SEQ ID NO: 53 and SEQ ID NO: 54, SEQ ID NO: 55 and SEQ ID NO: 56, SEQ ID NO: 57 and SEQ ID NO: 58, SEQ ID NO: 59 and SEQ ID NO: 60, SEQ ID NO: 61 and SEQ ID NO: 62, SEQ ID NO: 63 and SEQ ID NO: 64, SEQ ID NO: 65 and SEQ ID NO: 66, SEQ ID NO: 67 and SEQ ID NO: 68, SEQ ID NO: 69 and SEQ ID NO: 70, SEQ ID NO: 71 and SEQ ID NO: 72, SEQ ID NO: 73 and SEQ ID NO: 74, SEQ ID NO: 75 and SEQ ID NO: 76, and SEQ ID NO: 77 and SEQ ID NO: 78.

As used herein, the term “antibody” is used in the broadest sense and specifically covers monoclonai antibodies(including full length monocional antibodies), polyclonal antibodies, multi-specific antibodies (e.g., bispecific antibodies), and antibody fragments so long as they exhibit the desired biological activity. “Antibodies” (or “Abs”) and “immunoglobulins” (or “Igs”) are glycoproteins having the same structural characteristics. While antibodies exhibit binding specificity to a specific antigen, immunoglobulins include both antibodies and other antibody-like molecules which lack antigen specificity. Polypeptides of the latter kind are, for example, produced at low levels by the lymph system and at increased levels by myelomas.

As used herein, the term “epitope” means any antigenic determinant on an antigen to which the paratope of an antibody binds. Epitopic determinants usually consist of chemically active surface groupings of molecules such as amino acids or sugar side chains and usually have specific three dimensional structural characteristics, as well as specific charge characteristics.

As used herein, the term “native antibodies and immunoglobulins” are usually heterotetrameric glycoproteins of about 150,000 daltons, composed of two identical light (L) chains and two identical heavy (H) chains. Each light chain is linked to a heavy chain by one covalent disulfide bond (also termed a “VH/VL pair”), while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light- and heavy-chain variable domains. See, e.g., Clothia et al., J. Mol. Biol., 186:651 (1985); Novotny and Haber, Proc. Natl. Acad. Sci. U.S.A., 82:4592 (1985).

As used herein, the term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain and the heavy-chain variable domains. The more highly conserved portions of variable domains are called the framework (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen-binding site of antibodies. See, e.g., Kabat et al., Sequences of Proteins of Immunological Interest, Fifth Edition, National Institute of Health, Bethesda, Md. (1991). The constant domains are not involved directly in binding an antibody to an antigen, but exhibit various effector functions, such as participation of the antibody in antibody-dependent cellular toxicity. Variable region sequences of interest include the provided humanized variable region sequences for CD47 antibodies. For instance, 1A1 includes SEQ ID NO: 1 (heavy) and SEQ ID NO: 2 (light), 1F8 includes SEQ ID NO: 3 (heavy) and SEQ ID NO: 4 (light), and 2A11 includes SEQ ID NO: 5 (heavy) and SEQ ID NO: 6 (light).

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-combining sites and is still capable of cross-linking antigen. “Fv” is the minimum antibody fragment which contains a complete antigen-recognition and -binding site. In a two-chain Fv species, this region consists of a dimer of one heavy- and one light-chain variable domain in tight, non-covalent association. In a single-chain Fv species (scFv), one heavy- and one light-chain variable domain can be covalently linked by a flexible peptide linker such that the light and heavy chains can associate in a “dimeric” structure analogous to that in a two-chain Fv species. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site. See, e.g., Pluckthun, in The Pharmacology of Monoclonal Antibodies, Vol. 113, Rosenburg and Moore eds., Springer-Verlag, New York, pp. 269-315 (1994).

The Fab fragment also contains the constant domain of the light chain and the first constant domain (CH₁) of the heavy chain. Fab′ fragments differ from Fab fragments by the addition of a few residues at the carboxy terminus of the heavy chain CH₁ domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

There are five major classes of immunoglobulins: IgA, IgD, IgE, IgG, and IgM, and several of these can be further divided into subclasses (isotypes), e.g., IgG1, IgG2, IgG3, IgG4, IgA1, IgA2. The heavy-chain constant domains that correspond to the different classes of immunoglobulins are called α, δ, ε, γ, and μ, respectively. The subunit structures and three-dimensional configurations of different classes of immunoglobulins are well known.

As used herein, the term “antibody fragment”, and all grammatical variants thereof, are defined as a portion of an intact antibody comprising the antigen binding site or variable region of the intact antibody, wherein the portion is free of the constant heavy chain domains (i.e. CH2, CH3, and CH4, depending on antibody isotype) of the Fc region of the intact antibody. Examples of antibody fragments include Fab, Fab′, Fab′-SH, F(ab′)₂, and Fv fragments; diabodies; any antibody fragment that is a polypeptide having a primary structure consisting of one uninterrupted sequence of contiguous amino add residues (referred to herein as a “single-chain antibody fragment” or “single chain polypeptide”), including without limitation (1) single-chain Fv (scFv) molecules, (2) single chain polypeptides containing only one light chain variable domain, or a fragment thereof that contains the three CDRs of the light chain variable domain, without an associated heavy chain moiety, and (3) single chain polypeptides containing only one heavy chain variable region, or a fragment thereof containing the three CDRs of the heavy chain variable region, without an associated light chain moiety; and multi-specific or multivalent structures formed from antibody fragments. hi an antibody fragment comprising one or more heavy chains, the heavy chain(s) can contain any constant domain sequence (e.g. CH1 in the IgG isotype) found in a non-Fc region of an intact antibody, and/or can contain any hinge region sequence found in an intact antibody, and/or can contain a leucine zipper sequence fused to or situated in the hinge region sequence or the constant domain sequence of the heavy chain(s).

Unless specifically indicated to the contrary, the term “conjugate” used herein is defined as a heterogeneous molecule formed by the covalent attachment of one or more antibody fragment(s) to one or more polymer molecule(s), wherein the heterogeneous molecule is water soluble, i.e. soluble in physiological fluids such as blood, and wherein the heterogeneous molecule is free of any structured aggregate. A conjugate of interest is polyethylenglycol (PEG). In the context of the foregoing definition, the term “structured aggregate” refers to (1) any aggregate of molecules in aqueous solution having a spheroid or spheroid shell structure, such that the heterogeneous molecule is not in a micelle or other emulsion structure, and is not anchored to a lipid bilayer, vesicle or liposome; and (2) any aggregate of molecules in solid or insolubilized form, such as a chromatography bead matrix, that does not release the heterogeneous molecule into solution upon contact with an aqueous phase. Accordingly, the term “conjugate” as defined herein encompasses the aforementioned heterogeneous molecule in a precipitate, sediment, bioerodible matrix or other solid capable of releasing the heterogeneous molecule into aqueous solution upon hydration of the solid.

As used herein, the term “monoclonal antibody” (mAb) refers to an antibody obtained from a population of substantially homogeneous antibodies, i.e., the individual antibodies comprising the population are identical except for possible naturally occurring mutations that may be present in minor amounts, Monoclonal antibodies are highly specific, being directed against a single antigenic site. Each mAb is directed against a single determinant on the antigen. In addition to their specificity, the monoclonal antibodies are advantageous in that they can be synthesized by hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made in an immortalized B cell or hybridoma thereof, or may be made by recombinant DNA methods,

The monoclonal antibodies herein include hybrid and recombinant antibodies produced by splicing a variable (including hypervariable) domain of an CD47 antibody with a constant domain (e.g. “humanized” antibodies), or a light chain with a heavy chain, or a chain from one species with a chain from another species, or fusions with heterologous proteins, regardless of species of origin or immunoglobulin class or subclass designation, as well as antibody fragments (e.g., Fab, F(ab′)₂, and Fv), so long as they exhibit the desired biological activity.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity.

As used herein, an “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In some embodiments, the antibody will be purified (1) to greater than 75% by weight of antibody as determined by the Lowry method, and most preferably more than 80%, 90% or 99% by weight, or (2) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

As used herein, the term “epitope tagged” refers to a CD47 antibody fused to an “epitope tag”. The epitope tag polypeptide has enough residues to provide an epitope against which an antibody can be made, yet is short enough such that it does not interfere with activity of the CD47 antibody. The epitope tag preferably is sufficiently unique so that the antibody specific for the epitope does not substantially cross-react with other epitopes. Suitable tag polypeptides generally have at least 6 amino acid residues and usually between about 8-50 amino acid residues (preferably between about 9-30 residues). Examples include the c-myc tag and the 8F9, 3C7, 6E10, G4, 137 and 9E10 antibodies thereto (see, e.g., Evan et al., Mol. Cell, Biol., 5(12):3610-3616 (1985)); and the Herpes Simplex virus glycoprotein D (gD) tag and its antibody (see, e.g., Paborsky et al., Protein Engineering, 3(6):547-553 (1990)).

As used herein, the term “label” refers to a detectable compound or composition which is conjugated directly or indirectly to the antibody. The label may itself be detectable by itself (e.g., radioisotope labels or fluorescent labels) or, in the case of an enzymatic label, may catalyze chemical alteration of a substrate compound or composition which is detectable.

As used herein, the term “solid phase” refers to a non-aqueous matrix to which the antibody of the present invention can adhere. Examples of solid phases encompassed herein include those formed partially or entirely of glass (e.g. controlled pore glass), polysaccharides (e.g., agarose), polyacrylamides, polystyrene, polyvinyl alcohol and silicones. In certain embodiments, depending on the context, the solid phase can comprise the well of an assay plate; in others it is a purification column (e.g., an affinity chromatography column). This term also includes a discontinuous solid phase of discrete particles. See, e.g., U.S. Pat. No. 4,275,149.

The present invention also provides pharmaceutical compositions containing these CD47 antibodies and methods for treating diseases in a subject with these CD47 antibodies or pharmaceutical compositions.

As used herein, the term “treatment” or “treating” refers to both therapeutic treatment and prophylactic or preventative measures of a disease (such as cancer or a fibrotic disease). Those in need of treatment include those already with the disease as well as those in which the disease is to be prevented.

Examples of cancer include, but are not limited to, ovarian cancer, colon cancer, breast cancer, lung cancer, head and neck cancer, bladder cancer, colorectal cancer, pancreatic cancer, non-Hodgkin's lymphoma, acute lymphocytic leukemia, chronic lymphocytic leukemia, acute myeloid leukemia, chronic myelogenous leukemia, multiple myeloma, melanoma, leiomyoma, leiomyosarcoma, glioma, glioblastoma, myelomas, monocytic leukemias, B-cell derived leukemias, T-cell derived leukemias, B-cell derived lymphomas, T-cell derived lymphomas, and solid tumors. The fibrotic disease can be, e.g., myocardial infarction, angina, osteoarthritis, pulmonary fibrosis, asthma, cystic fibrosis, bronchitis, or asthma.

As used herein, the term “subject” for purposes of treatment refers to any animal classified as a mammal, including humans, domestic and farm animals, and zoo, sports, or pet animals, such as dogs, horses, cats, cows, etc. Preferably, the mammal is human.

The CD47 antibodies of this invention can also be used in vitro and in vivo to monitor the course of CD47 disease therapy. Thus, for example, by measuring the increase or decrease in the number of cells expressing CD47, particularly cancer cells expressing CD47, it can be determined whether a particular therapeutic regimen aimed at ameliorating disease is effective.

The CD47 antibodies of this invention may be used in vitro in immunoassays in which they can be utilized in liquid phase or bound to a solid phase carrier. In addition, the CD47 antibodies in these immunoassays can be detectably labeled in various ways. Examples of types of immunoassays which can utilize monoclonal antibodies of the invention are flow cytometry, e.g, FACS, MACS, immunohistochemistry, competitive and non-competitive immunoassays in either a direct or indirect format. Detection of the antigens using the CD47 antibodies of this invention can be done utilizing immunoassays which are run in either the forward, reverse, or simultaneous modes, including immunohistochemical assays on physiological samples. Those of skill in the art will know, or can readily discern, other immunoassay formats without undue experimentation.

The CD47 antibodies of the invention can be bound to many different carriers and used to detect the presence of CD47 expressing cells, Examples of well-known carriers include glass, polystyrene, polypropylene, polyethylene, dextran, nylon, amylases, natural and modified celluloses, polyacrylamides, agaroses and magnetite. The nature of the carrier can be either soluble or insoluble for purposes of the invention. Those skilled in the art will know of other suitable carriers for binding monoclonal antibodies, or will be able to ascertain such, using routine experimentation.

There are many different labels and methods of labeling known to those of ordinary skill in the art, which find use as tracers in therapeutic methods, for use in diagnostic methods, and the like. For diagnostic purposes a label may be covalently or non-covalently attached to an antibody of the invention or a fragment thereof, including fragments consisting or comprising of CDR sequences. Examples of the types of labels which can be used in the present invention include enzymes, radioisotopes, fluorescent compounds, colloidal metals, chemiluminescent compounds, and bio-luminescent compounds. Those of ordinary skill in the art will know of other suitable labels for binding to the monoclonal antibodies of the invention, or will be able to ascertain such, using routine experimentation. Furthermore, the binding of these labels to the monoclonal antibodies of the invention can be done using standard techniques common to those of ordinary skill in the art.

In some embodiments, a CD47 antibody of this invention is attached to a nanoparticle, e.g. for use in imaging. Useful nanoparticles are those known in the art, for example including without limitation, Raman-silica-gold-nanoparticle (R—Si—Au-NP). The R—Si—Au-NPs consist of a Raman organic molecule, with a narrow-band spectral signature, adsorbed onto a gold core. Because the Raman organic molecule can be changed, each nanoparticles can carry its own signature, thereby allowing multiple nanoparticles to be independently detected simultaneously by multiplexing. The entire nanoparticle is encapsulated in a silica shell to hold the Raman organic molecule on the gold nanocore. Optional polyethylene glycol (PEG)-ylation of R—Si—Au-NPs increases their bioavailability and provides functional “handles” for attaching targeting moieties. See, e.g., Thakor et al (2011), Sci. Transl. Med., 3(79):79ra33; Jokerst et al. (2011) Small., 7(5):625-33; Gao et al. (2011) Biomaterials, 32(8):2141-8.

For purposes of the invention, CD47 may be detected by the CD47 antibodies of this invention when present in biological fluids and on tissues, in vivo or in vitro. Any sample containing a detectable amount of CD47 can be used. A sample can be a liquid such as urine, saliva, cerebrospinal fluid, blood, serum and the like, or a solid or semi-solid such as tissues, feces, and the like, or, alternatively, a solid tissue such as those commonly used in histological diagnosis.

Another labeling technique which may result in greater sensitivity consists of coupling the antibodies to low molecular weight haptens. These haptens can then be specifically detected by means of a second reaction. For example, it is common to use haptens such as biotin, which reacts with avidin, or dinitrophenol, pyridoxal, or fluorescein, which can react with specific anti-hapten antibodies.

As a matter of convenience, a CD47 antibody of this invention can be provided in a kit, i.e., a packaged combination of reagents in predetermined amounts with instructions for performing the diagnostic assay. Where the antibody is labeled with an enzyme, the kit will include substrates and cofactors required by the enzyme (e.g., a substrate precursor which provides the detectable chromophore or fluorophore). In addition, other additives may be included such as stabilizers, buffers (e.g., a block buffer or lysis buffer) and the like. The relative amounts of the various reagents may be varied widely to provide for concentrations in solution of the reagents which substantially optimize the sensitivity of the assay. Particularly, the reagents may be provided as dry powders, usually lyophilized, including excipients which on dissolution will provide a reagent solution having the appropriate concentration.

Therapeutic formulations comprising one or more antibodies of the invention are prepared for storage by mixing the antibody having the desired degree of purity with optional physiologically acceptable carriers, excipients or stabilizers (see, e.g., Remington's Pharmaceutical Sciences, 16th edition, Osol, A. Ed. (1980)), in the form of lyophilized formulations or aqueous solutions. The antibody composition will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “therapeutically effective amount” of the antibody to be administered will be governed by such considerations, and is the minimum amount necessary to prevent the CD47 associated disease.

The therapeutic dose may be at least about 0.01 82 g/kg body weight, at least about 0.05 ug/kg body weight; at least about 0.1 μg/kg body weight, at least about 0.5 μg/kg body weight, at least about 1 μg/kg body weight, at least about 2.5 μg/kg body weight, at least about 5 μg/kg body weight, and not more than about 100 μg/kg body weight. It will be understood by one of skill in the art that such guidelines will be adjusted for the molecular weight of the active agent, e.g. in the use of antibody fragments, or in the use of antibody conjugates. The dosage may also be varied for localized administration, e.g. intranasal, inhalation, etc., or for systemic administration, e.g., intraperitoneal (I.P.), intravenous (I.V.), intradermal (I.D.), intramuscular (I.M.), and the like,

A CD47 antibody of this invention needs not be, but is optionally formulated with one or more agents that potentiate activity, or that otherwise increase the therapeutic effect. These are generally used in the same dosages and with administration routes as used hereinbefore or about from 1 to 99% of the heretofore employed dosages.

Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). Formulations to be used for in vivo administration must be sterile. This is readily accomplished by filtration through sterile filtration membranes.

The active ingredients containing CD47 antibodies may also be entrapped in microcapsule prepared, e.g., by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed, (1980).

A CD47 antibody or pharmaceutical composition of this invention can be administered by any suitable means, including parenteral, subcutaneous, intraperitoneal, intrapulmonary, and intranasal. Parenteral infusions include intramuscular, intravenous, intraarterial, intraperitoneal, or subcutaneous administration. In addition, the anti-CD47 antibody is suitably administered by pulse infusion, particularly with declining doses of the antibody.

For the prevention or treatment of disease, the appropriate dosage of antibody will depend on the type of disease to be treated, as defined above, the severity and course of the disease, whether the antibody is administered for preventive purposes, previous therapy, the patient's clinical history and response to the antibody, and the discretion of the attending physician. The antibody is suitably administered to the patient at one time or over a series of treatments.

In another embodiment of the invention, an article of manufacture containing materials useful for the treatment of the disorders described above is provided. The article of manufacture comprises a container and a label. Suitable containers include, for example, bottles, vials, syringes, and test tubes. The containers may be formed from a variety of materials such as glass or plastic. The container holds a composition which is effective for treating the condition and may have a sterile access port (e,g., the container may be an intravenous solution bag or a vial having a stopper pierceable by a hypodermic injection needle). The active agent in the composition is the anti-CD47 antibody. The label on, or associated with, the container indicates that the composition is used for treating the condition of choice. The article of manufacture may further comprise a second container comprising a pharmaceutically-acceptable buffer, such as phosphate-buffered saline, Ringer's solution and dextrose solution. It may further include other materials desirable from a commercial and user standpoint, including other buffers, diluents, filters, needles, syringes, and package inserts with instructions for use.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### Establishment of Phage Library

CD47 is a 50 kDa membrane receptor that has extracellular N-terminal IgV domain, five transmembrane domains, and a short C-terminal intracellular tail. Human CD47-IgV domain protein conjugated with human Fc or Biotinylated human CD47-IgV domain protein (ACROBiosystems) was used as antigen for phage library panning.

The phage library was constructed using phagemid vectors which consisted of the antibody gene fragments that were amplified from spleens or bone marrows of >50 healthy human subjects. The antibody format is single chain variable fragment (VH+linker+VL). The library size was 1.1×1010 and the sequence diversity was analyzed as follows. For the 62 clones picked up from the library and further sequenced, 16 sequences have truncation, frameshift or amber codon; 46 sequences have full length scFv of which all the HCDR3 sequences are unique. In the 46 full length scFv, 13 sequences have lambda light chain and 33 sequences have kappa light chain.

### Phage Panning and Clone Selection

To obtain phage clones that specifically bind to the human CD47-IgV domain, two methods for phage panning were used.

### 1. Phage Library Immunotube Panning Against Human CD47-IgV

In this method, the phage libraries developed as described above were first incubated in casein-coated immunotube for 2 hours. The human CD47-IgV-Fc fusion protein was used for first round of panning. Unbound phages were removed by washing with PBST for 5-20 times. The bound phages were eluted with freshly prepared 100 mM Triethylamine solution and neutralized by addition a Tris-HCl buffer, to become the first output phage pools. This first output phage pool was rescued through infection of E. Coli TG-1 cells for amplification, followed by the second round of panning using biotinylated human CD47-IgV as antigen. The bound phages were eluted in the same process and became the second output phage pool which was then rescued and then again followed by the third round of panning using human CD47-IgV-Fc fusion protein as antigen. The bound phages then became the third output phage pool and underwent the fourth round of panning using biotinylated human CD47-IgV.

### 2. Phage Library Solution Panning Against Human CD47-IgV

In this second method, the phage libraries were first incubated in casein-blocked 100 μL streptavdin-magnetic beads to deplete streptavdin beads binders. The streptavidin-magnetic beads and AG0084-hulgG1/k were used for negative depletion. The depleted library was rescued, which was followed by the second round of panning using biotinylated human CD47-IgV as antigens and further underwent negative depletion with casein blocked streptavdin-magnetic beads. The unbound phages were removed by washing with PBST for 5-20 times. The bound phages were eluted with a freshly prepared 100 mM Triethylamine solution, neutralized by addition of a Tris-HCl buffer, and then rescued, which was followed by the third round of panning using human CD47-IgV-Fc fusion protein and depleted with AG0084-hulgG1/k. The bound phages then become the third output phage pool and underwent the fourth round of panning using biotinylated human CD47-IgV and negative depletion with casein blocked streptavdin-magnetic beads.

After this process, multiple phage clones that specifically bound to the human CD47-IgV domain were obtained and enriched. They were then diluted and plated to grow at 37° C. for 8 hours and captured by anti-kappa antibody-coated filter overnight. Biotinylated human CD47-IgV (50 nM) and NeutrAvidin-AP conjugate (1:1000 dilution) were applied to the filter to detect the positively bound phage clones. Positive phage plaques were picked and eluted into 100 μL of phage elution buffer. About 10-15 μL eluted phages were used to infect 1 mL XL1 blue cells to make high titer phage (HT) for Phage single point ELISA (SPE). The positive single clones picked from the filer lift were subjected to the binding of human CD47-IgV-Fc fusion protein and biotinylated human CD47-IgV domain protein. These positive single clones were also sequenced for their VH and VL genes. All the positive hits with unique VH and VL genes were cloned into expression vectors pFUSE2ss-CLIg-hk (light chain, InvivoGen, Cat No. pfuse2ss-hclk) and pFUSEss-CHIg-hG1 (heavy chain, InvivoGen, Cat No. pfusess-hchgl). The antibodies were expressed in HEK293 cells and purified by Protein A Plus Agarose.

### Affinity Maturation of CD-47 Antibodies

Binding affinity of the CD-47 antibodies of this invention can be improved by in vitro affinity maturation, e.g., by site-specific randomized mutation, which resulted in mutated sequences that are also within the scope of this invention.

For example, BiaCore analysis of 1F8, a CD47 antibody of this invention, showed a binding affinity (KD) of 2.8 nM with a high dissociation rate of 1.04E-03 1/s, which could be improved by in vitro affinity maturation. An extensive analysis of the CDR sequence of heavy chain and light chain of 1F8 identified several residues in HCDR1 and LCDR1 regions that could be randomized mutated. Therefore, the random mutagenesis libraries can be constructed and introduced into the specific residues to generate a variety of new sequences. The CDR mutagenesis libraries are panned using biotinylated soluble CD47 ECD in solution phase under the equilibrium condition. After multiple rounds of panning with reduced antigen concentration, enriched output binders are selected for the binding ELISA test and subsequent converted into full IgGs which are subjected to the BiaCore analysis to specifically select for the off-rate improved sequence. Through this screening process, antibody molecules of this invention can be constructed for overall best properties for clinical applications.

### EXAMPLE 1. ELISA SCREENING OF PHAGE CLONES BINDING TO RECOMBINANT CD47-ECD PROTEIN

Recombinant human CD47-Fc fusion protein (Acrobiosystems) was coated at 2 ug/mL in phosphate buffer saline (PBS) onto microtiter plates for 2 hours at the room temperature (RT). After coating of antigen, the wells were blocked with PBS/0.05% Tween (PBST) with 1% BSA for 1 hour at the room temperature (RT). After washing of the wells with PBST, purified phages from single clones were added to the wells and incubated for 1 hour at RT. For detection of the binding phage clones, the HRP conjugated secondary antibodies against M13 (Jackson Immuno Research) were added, followed by the addition of fluorogenic substrates (Roche). Between all incubation steps, the wells of the plate were washed with PBST three times. Fluorescence was measured in a TECAN Spectrafluor plate reader. The positive phage clones were selected for sequencing of the heavy chain and light chain genes.

All of the tested CD47 antibodies of this invention showed good binding activities for recombinant human CD47-Fc fusion protein.

### EXAMPLE 2. ELISA ANALYSIS OF ANTIBODIES BLOCKING THE INTERACTION OF CD47 TO SIRPα

Recombinant human CD47/mouse Fc fusion protein or biotinylated CD47 protein (Acrobiosystems) was coated at 1 ug/mL in PBS onto microtiter plates for 2 hours at RT. After coating of antigen the wells were blocked with PBS/0.05% Tween (PBST) with 1% BSA for 1 hour at RT. After washing of the wells with PBST, the antibodies diluted in PBS were added to the wells (5 ug/mL) and incubated for 1 hour at RT. For detection of the binding antibodies, the HRP conjugated secondary antibodies against human Fc (Jackson Immuno Research) were added, followed by the addition of fluorogenic substrates (Roche). Between all incubation steps, the wells of the plate were washed with PBST three times. Fluorescence was measured in a TECAN Spectrafluor plate reader.

All of the tested CD47 antibodies of this invention showed good binding activities for recombinant human CD47-Fc fusion protein and biotinylated CD47 protein.

### EXAMPLE 3. ELISA ANALYSIS OF ANTIBODIES BLOCKING THE INTERACTION OF CD47 TO SIRPα

Recombinant CD47-Fc fusion protein (Acrobiosystems) was coated at 1 ug/mL in PBS onto microtiter plates for 16 hours at 4° C. After blocking for 1 hour with 1% BSA in PBST at RT, 1 ug/ml of SIRPα-His protein was added either in the absence or presence of CD47 antibodies (10 ug/mL) at RT for 1 hour. Plates were subsequently washed three times and incubated with an HRP-conjugated anti-His secondary antibody for 1 hour at RT. After washing, the TMB solution was added to each well for 30 minutes and the reaction was stopped with 2.0 M H₂SO₄, and OD was measured at 490 nm.

All of the tested CD47 antibodies of this invention effectively blocked the CD47 protein-SIRPα binding.

### EXAMPLE 4. DOSE-DEPENDENT RESPONSE OF CD47 ANTIBODIES BINDING TO MONOMERIC CD47-ECD

After direct binding and competition screening, a CD47 antibody of this invention 1F8 was selected for this test, in comparison with two existing reference antibodies. Biotinylated CD47 protein (Acrobiosystems) was coated at 1 ug/mL in PBS onto microtiter plates for 2 hours at RT. After coating of antigen, the wells were blocked with PBS/0.05% Tween (PBST) with 1% BSA for 1 hour at RT. After washing of the wells with PBST, different concentrations of CD47 antibodies were added to the well and incubated for 1 hour at RT. For detection of the binding antibodies, the HRP conjugated secondary antibodies against human Fc (Jackson Immuno Research) were added followed by the addition of fluorogenic substrates (Roche). Between all incubation steps, the wells of the plate were washed with PBST three times. Fluorescence was measured in a TECAN Spectrafluor plate reader.

Reference antibodies 5F9 and 2A1 was produced according to the sequence of Hu5F9 and CC-90002 as disclosed by researchers at Stanford University, Inhibrx LLC, and Celgene Corp. (see, e.g., US Pat. No. 9,017,675 B2, U.S. Pat. No. 9,382,320, U.S. Pat. No. 9,221,908, US Pat. Application Pub. No. 2014/0140989 and WO 2016/109415) and used for the same study.

As shown in FIG. 1, all three antibodies (1F8, 5F9, and 2A1) showed similar binding activities to monomeric CD47-ECD.

### EXAMPLE 5. DOSE-DEPENDENT RESPONSE OF CD47 ANTIBODIES BINDING TO DIMERIC CD47-ECD

The three CD47 antibodies used in Example 4 (i.e., 1F8, 5F9, and 2A1) were also used in this study.

CD47/mouse Fc fusion protein (Acrobiosystems) was coated at 1 ug/ml in PBS onto microtiter plates for 2 hours at RT. After coating of antigen the wells were blocked with PBS/0.05% Tween (PBST) with 1% BSA for 1 hour at RT. After washing of the wells with PBST, different concentrations of anti-CD47 antibodies were added to the well and incubated for 1 at RT. For detection of the binding antibodies, the HRP conjugated secondary antibodies against human Fc (Jackson Immuno Research) were added followed by the addition of fluorogenic substrates (Roche). Between all incubation steps, the wells of the plate were washed with PBST three times. Fluorescence was measured in a TECAN Spectrafluor plate reader.

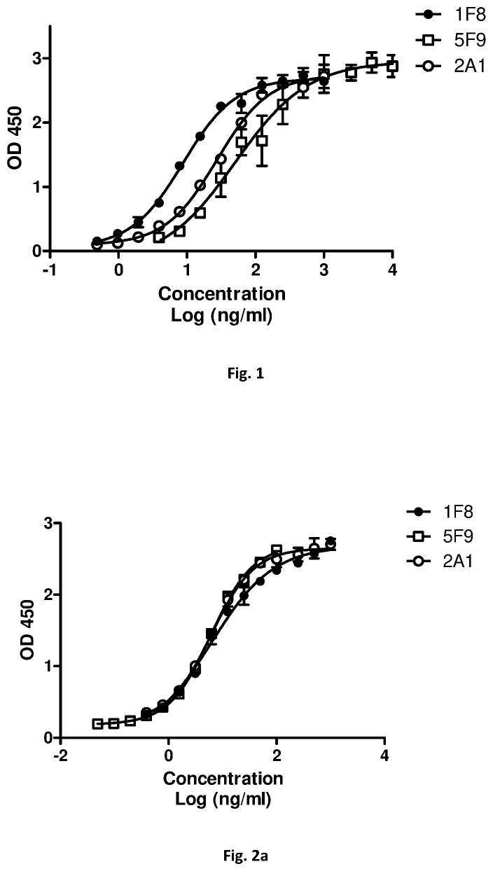

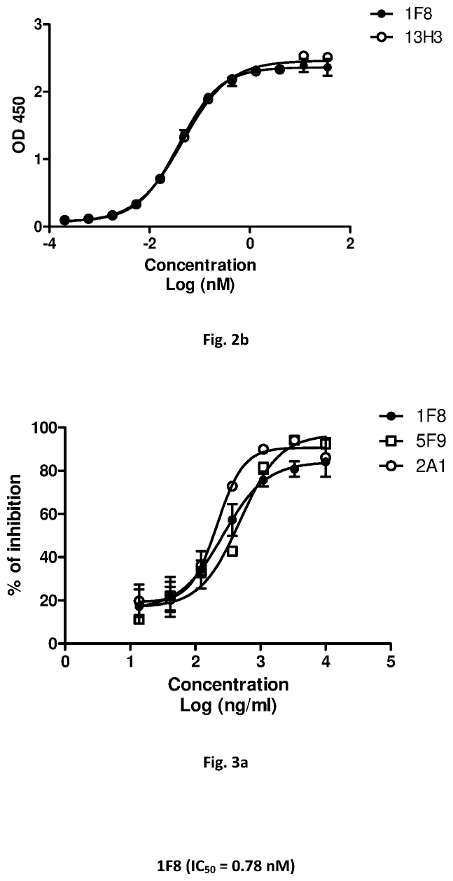

Likewise, as shown in FIG. 2a, among the three tested antibodies 1F8, 5F9, and 2A1, all of them showed similar binding activities to dimeric CD47-ECD.

Another binding study was conducted to compare the binding affinity of two antibodies of this invention, i.e., 1F8 and 13H3, to recombinant CD49-ECD. As shown in FIG. 2b, these two antibodies also exhibited similar binding activities in a dose-dependent manner, with EC₅₀ being 0.038 nM for 1F8 and 0.045 nM for 13H3.

### EXAMPLE 6. DOSE-DEPENDENT RESPONSE OF CD47 ANTIBODIES BLOCKING THE BINDING OF CD47 TO SIRPα

Three CD47 antibodies (i.e., 1F8, 5F9, and 2A1) were also used in this study.

Recombinant CD47-Fc fusion protein (Acrobiosystems) was coated at 1 ug/ml in PBS onto microtiter plates for 16 hours at 4° C. After blocking for 1 h with 1% BSA in PBST at RT, 1 ug/mL of SIRPa-His protein was added either in the absence or presence of different concentrations of anti-CD47 antibodies at RT for 1 h. Plates were subsequently washed three times and incubated with an HRP-conjugated anti-His secondary antibody for 1 h at RT. After washing, the TMB solution was added to each well for 30 min and the reaction was stopped with 2M H₂SO₄, and OD was measured at 490 nm.

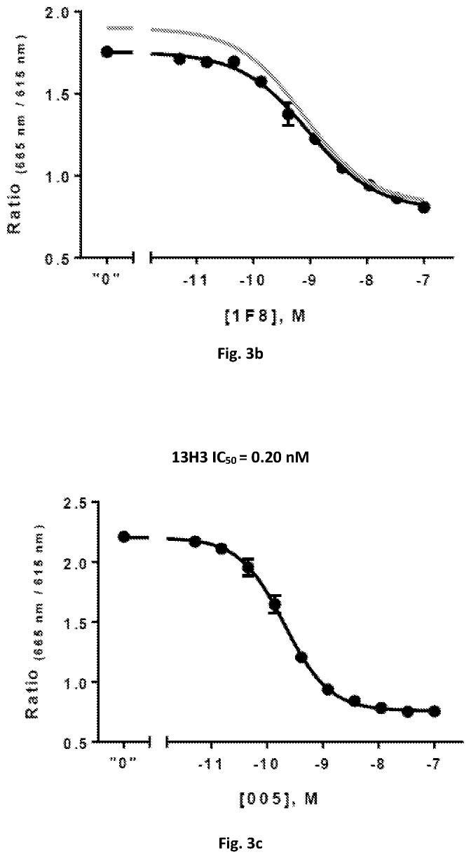

Again, as shown in FIG. 3a, all three antibodies showed similar activities in blocking the binding of CD47 to SIRPα.

Another study was conducted to compare the ability of two CD47 antibodies of this invention 1F8 and 13H3 to block the binding of CD47 to SIRPα. As shown in FIG. 3b and FIG. 3c, these two antibodies also exhibited similar blocking activities in a dose-dependent manner, with IC₅₀ being 0.78 nM for 1F8 and 0.20 nM for 13H3.

### EXAMPLE 7. DOSE-DEPENDENT RESPONSE OF CD47 ANTIBODIES BINDING TO CD47⁺ RAJI CELLS

Three CD47 antibodies (i.e., 1F8, 5F9, and 2A1) were also used in this study.

Raji cells which endogenously express human CD47 on the surface were stained with different concentrations of 1F8, 5F9 and 2A1 antibodies at 4° C. for 30 minutes. Then, the cells were washed with PBS three times, followed by incubation with APC-labeled anti-human Fc specific antibody (Invitrogen) at 4° C. for 30 minutes. Binding was measured using a FACSCanto (Becton-Dickinson).

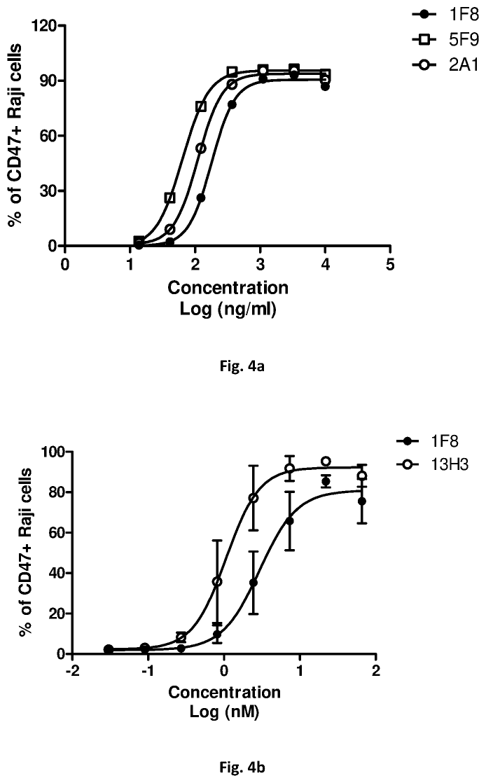

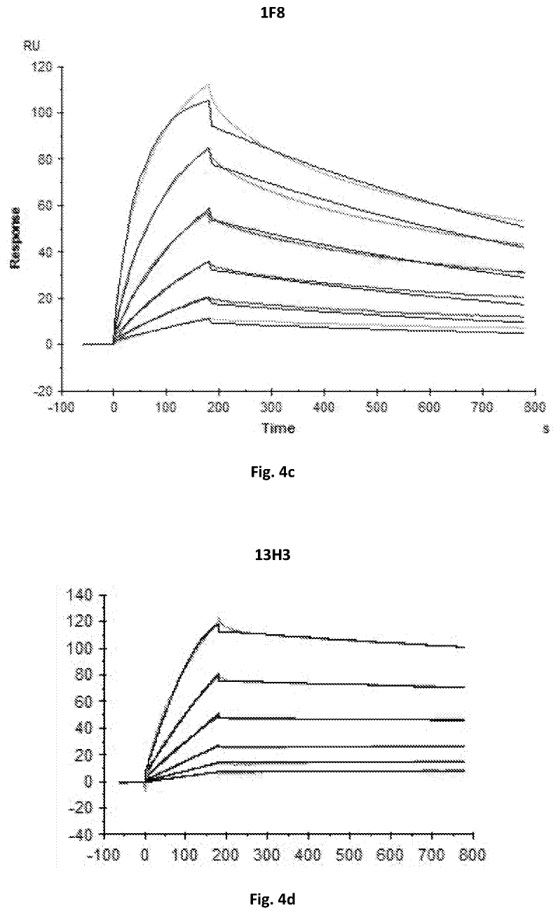

As shown in FIG. 4a, all three antibodies showed similar activities in binding to CD47⁺ Raji cells, following the same dose-dependent pattern.

Another study was conducted to compare the ability of two CD47 antibodies of this invention 1F8 and 13H3 to bind to CD47-bearing Raji cells. As shown in FIG. 4b, 13H3 exhibited stronger affinity than 1F8 in binding CD47-bearing Raji cells, with EC₅₀ being 2.95 nM for 1F8 and 1.06 nM for 13H3.

FIG. 4c and FIG. 4d show the binding kinetics of 1F8 and 13H3, respectively, as measured by Biocore analysis; and FIG. 4e shows the data.

### EXAMPLE 8. STUDY OF PHAGOCYTOSIS OF TUMOR CELLS BY HUMAN MACROPHAGE (MCP)

Three CD47 antibodies (i.e., 1F8, 5F9, and 2A1) were also used in this study.

PBMCs were isolated from human blood, and the monocytes were differentiated into macrophages for 6 days. The monocyte derived macrophages (MDMs) were scraped and re-plated in 24-well dishes and allowed to adhere for 24 hours. The human tumor cell line Raji which endogenously expressed CD47 were chosen as target cells and labeled with 1 uM CFSE for 10 minutes, then added to MDMs at a ratio of 5:1 tumor cells per phagocyte and CD47 antibodies was added at various doses. After incubation for 3 hours, non-phagocytosed target cells were washed away with PBS and the remaining phagocytes were scraped off, stained with macrophage marker CD14 antibody, and analyzed by flow cytometry. Phagocytosis was measured by gating on CD14⁺ cells and then assessing the percent of CFSE⁺ cells.

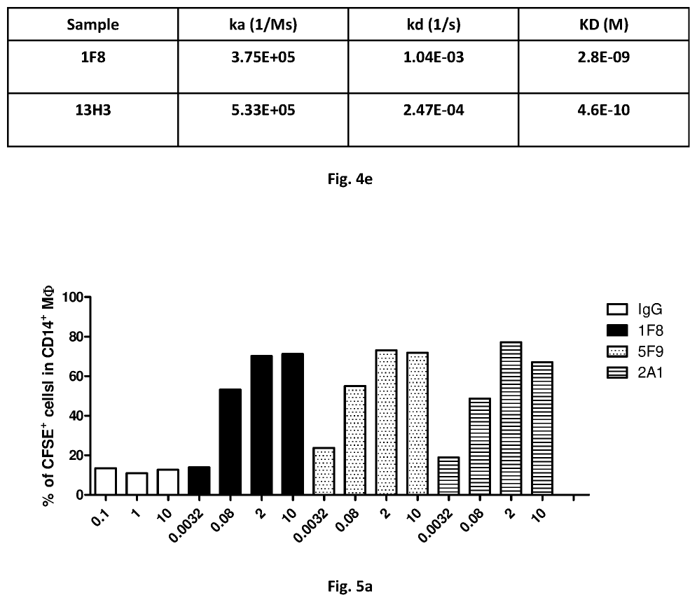

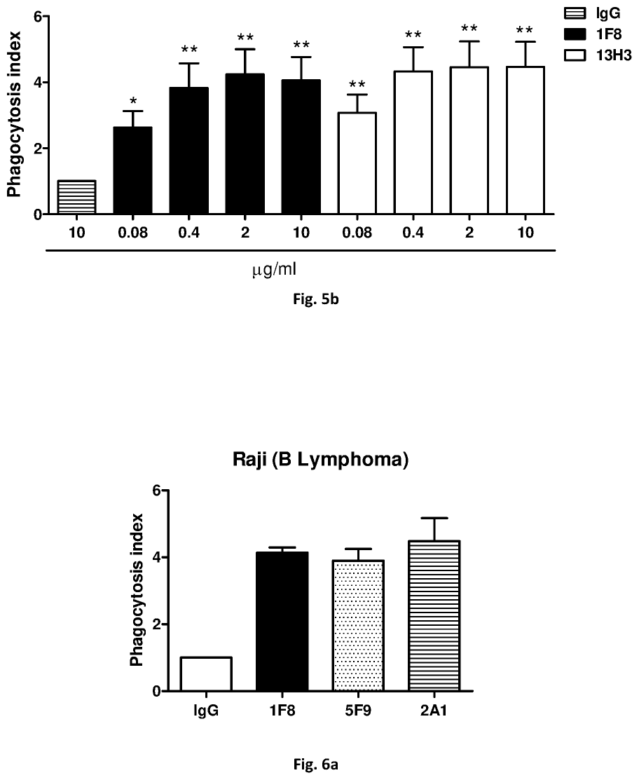

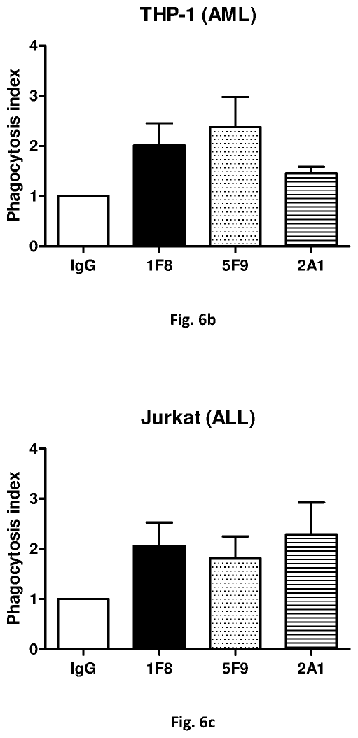

As shown in FIG. 5a, all these three tested antibodies (i.e., 1F8, 5F9, and 2A1) showed similar activities in promoting phagocytosis of tumor cells by human MΦ. FIGS. 6a, 6b, and 6c show the macrophage-mediated phagocytosis of three different human blood cancer cell lines, triggered by the three CD47 antibodies.

Another study was conducted to compare the ability of two CD47 antibodies of this invention 1F8 and 13H3 to promote phagocytosis of tumor cells by human MΦ. As shown in FIG. 5b, 13H3 and 1F8 exhibited similar abilities with 13H3 slightly stronger phagocytosis at some concentrations.

### EXAMPLE 9. RBC-SPARING PROPERTY IN RBC AGGLUTINATION ASSAY

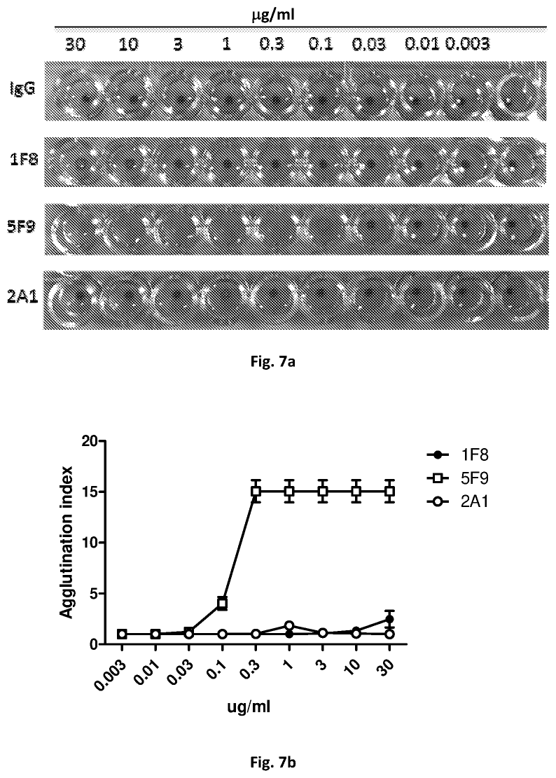

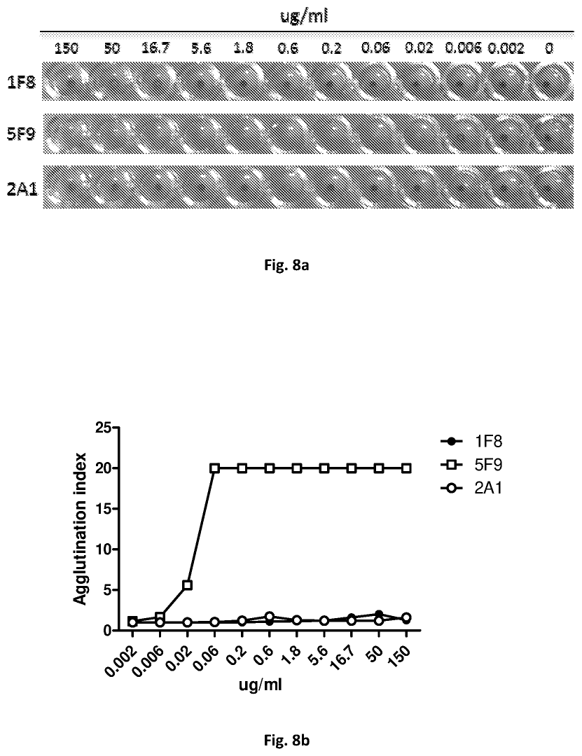

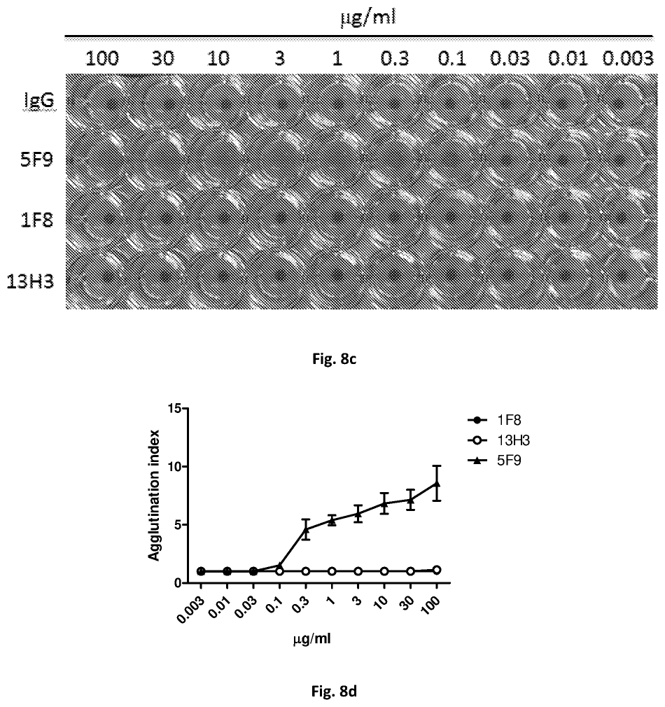

Human RBCs were diluted to 10% in PBS and incubated at 37° C. for 2 hours with a titration of CD47 antibodies in a round bottom 96-well plate. Evidence of hemagglutination is demonstrated by the presence of non-settled RBCs, appearing as a haze compared to a punctuate red dot of non-hemagglutinated RBCs (see FIGS. 7a and 8a). The graphs in FIGS. 7b and 8b show the quantitation of the hemagglutination assay, denoted “agglutination index” determined by quantitating the area of the RBC pellet in the presence of the antibody, normalized to that of IgG control.

As shown in FIGS. 7a, 7b, 8a, and 8b, while CD47 antibody 5F9 already showed significant RBC agglutination at a concentration of or higher than 0.1 ug/uL, CD47 antibodies 1F8 and 2A1 resulted in essentially no RBC agglutination at the tested concentrations up to 30 ug/uL (FIGS. 7a and 7b) or even up to 150 ug/mL (FIGS. 8a and 8b).

Likewise, FIGS. 8c and 8d show that CD47 antibodies of this invention (i.e., 1F8 and 13H3) resulted in essentially no RBC agglutination at the tested concentrations up to 150 ug/mL, whereas CD47 antibody 5F9 already showed significant RBC agglutination at a concentration of or higher than 0.1 ug/uL.

### EXAMPLE 10. RBC BINDING ASSAY

Binding of CD47 antibodies against human RBCs was examined by flow cytometry. Human RBCs were incubated with CD47 antibodies (10 ug/mL) at 4° C. for 1 hour, followed by the addition of APC-conjugated secondary antibody at 4° C. for 30 minutes.

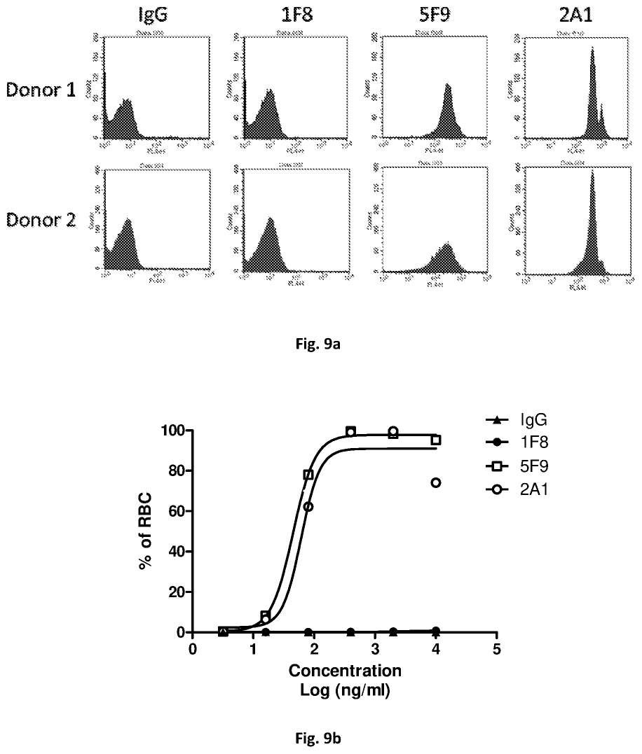

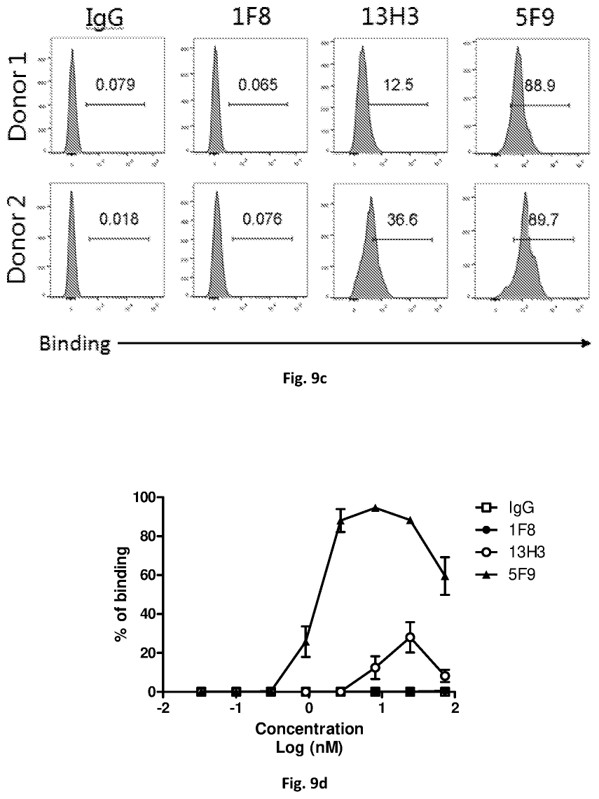

As shown in FIGS. 9a and 9b, surprisingly, CD47 antibody of this invention 1F8 did not bind to RBC while reference CD47 antibodies 5F9 and 2A1 did at the tested concentrations.

Likewise, FIGS. 9c and 9d show that while 1F8 resulted in no RBC binding at the tested concentrations, 13H3 only resulted in very low RBC binding at the tested concentrations.

### EXAMPLE 11. RBC AGGLUTINATION ASSAY

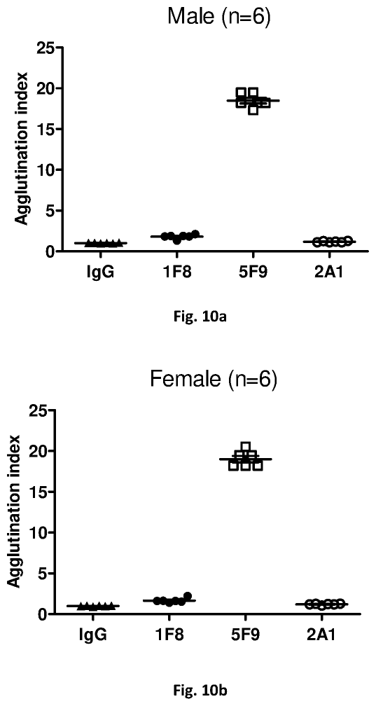

RBCs were collected from six male and six female healthy individuals for the analysis of RBC agglutination by the addition of CD47 antibodies. FIGS. 10a and 10b show the titration results of the hemagglutination assay, which is denoted “agglutination index” as determined by measuring the area of the RBC pellets in the presence of the antibody, normalized to that of IgG control or reference antibody.

### EXAMPLE 12. PLATELET BINDING ASSAY

Binding of CD47 antibodies of this invention against human platelets was examined by flow cytometry. Human peripheral whole blood was incubated with test CD47 antibodies of this invention (at 10 ug/mL) or SIRPα-Ig fusion and CD61 was stained as a surface marker for platelets. The binding of CD47 antibodies or SIRPα-Ig fusion was measured by gating on the CD61 positive population (platelet) and further examining the percentages of CD47 or SIRPα-Ig fusion binding.

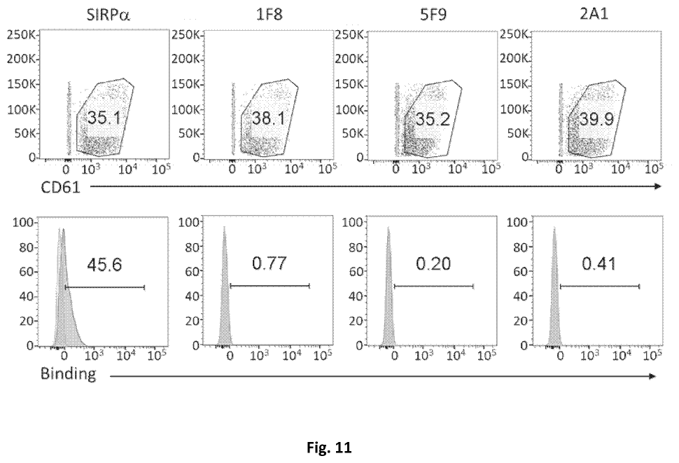

As shown in FIG. 11, tested CD47 antibodies of this invention did not appreciably bind to human platelets whereas SIRPαproteins did.

### EXAMPLE 13. CYNO RBC AGGLUTINATION ASSAY

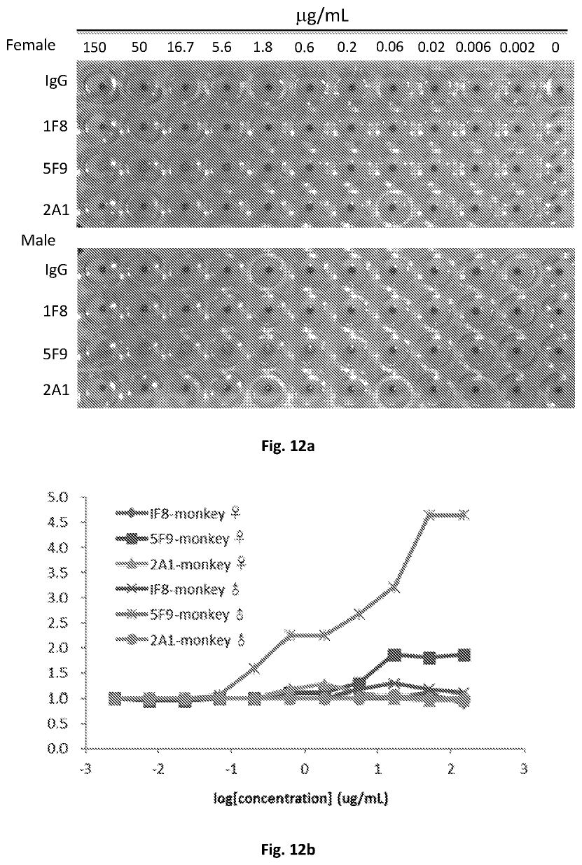

RBCs from male and female cyno monkey were diluted to 10% in PBS and incubated at 37° C. for 2 hours with the indicated concentrations of CD47 antibodies in a round bottom 96-well plate. Evidence of hemagglutination was demonstrated by the presence of non-settled RBCs, appearing as a haze compared to a punctuate red dot of non-hemagglutinated RBCs, as shown in FIG. 12a. FIG. 12b shows the titration results of the hemagglutination assay, which is denoted “agglutination index” as determined by measuring the area of the RBC pellets in the presence of the antibody, normalized to that of IgG control.

The data show that the tested CD47 antibodies of this invention did not appreciably induce cyno RBC agglutination in vitro.

### EXAMPLE 14. PHAGOCYTOSIS OF PRIMARY HUMAN AML CELLS BY CD47 ANTIBODIES

Primary PBMCs from AML patient (AML-PB003F) were labeled with 1 uM CFSE for 10 minutes, then added to MDMs at a ratio of 5:1 tumor cells per phagocyte and the indicated CD47 antibodies was added at various concentrations. After 3-hr incubation, non-phagocytosed target cells were washed away with PBS and the remaining phagocytes were scraped off, stained with a CD14 antibody, and analyzed by flow cytometry. Phagocytosis was measured by gating on CD14+ cells and then assessing the percentage of CFSE+ cells. Phagocytosis was measured as previously mentioned.

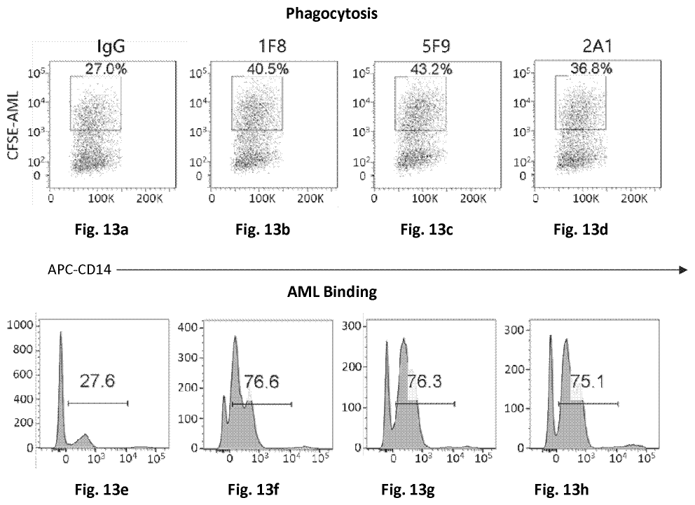

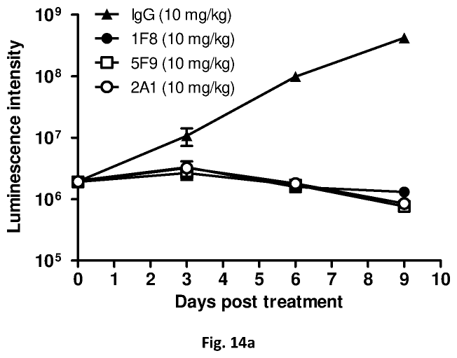

As shown in FIGS. 13a-13h, the tested CD47 antibodies of this invention all showed significant AML binding capabilities (greater than 75%) and phagocytosis capabilities (at least 36%), all of which are much higher than the reference CD47 antibody used in the same essay.

### EXAMPLE 15. IN VIVO EFFICACY OF 1F8 USING LUCIFERASE-RAJI XENOGRAFT MODEL (CDX)

NSG mice were engrafted with Raji Luc-EGFP at a concentration of 1 million cells/mouse via tail vein injection. They were imaged in vivo to determine the level of engraftment five days post engraftment. Treatment of CD47 antibodies (i.e., 1F8, 5F9, and 2A1) started from the same day at a dose of 10 mg/kg. All mice were injected every other day via intraperitoneal injection. Mice were imaged in vivo via IVIS Lumina III imaging system at the following time points: Day 0 of antibody treatment, Day 2 of treatment, Day 6 of treatment, and Day 9 of treatment. The tumor growth in the mice was measured by the analysis of bioluminescent radiance through in vivo live imaging system.

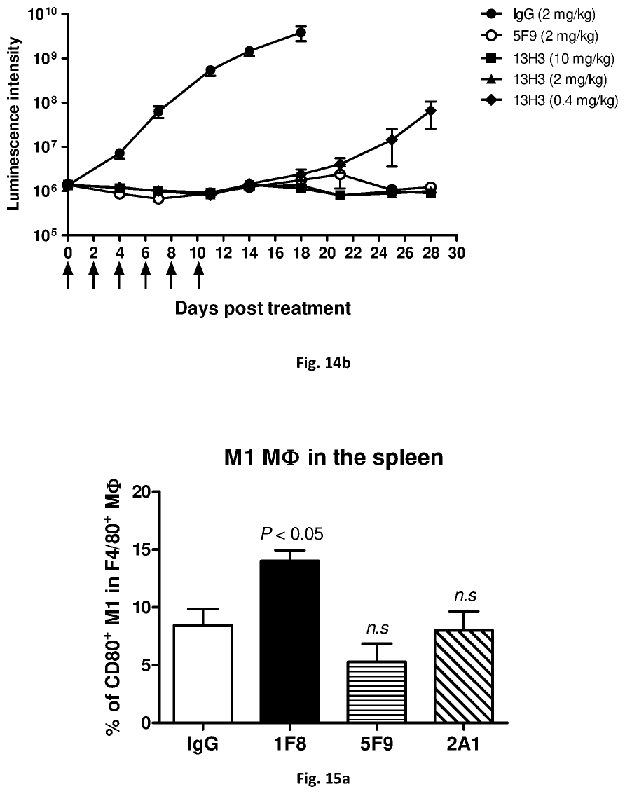

As can be seen in FIG. 14a, the analysis of bioluminescent radiance shows that the tumors in the mice barely grew within the first three days after the treatments with the tested CD47 antibody of this invention (i.e., 1F8) and the tumors reduced from day 6 after the treatments. By comparison, the tumors in the mice treated with reference CD47 antibody continued to grow during the same treatment period.

Similarly, FIG. 14b shows that the CD47 antibody 13H3 was also effective in vivo in Raji xenograph model at different test concentrations.

In the end of Raji-xenograft study, all the mice were euthanized by the use of CO₂ for rodent euthanasia. The splenocytes from four groups of mice were isolated and analyzed for the percentage of MI. macrophages (% of CD80 positive in F4/80 positive macrophages) and M2 macrohpages (% of CD206 positive in F4/80 positive macrophages) by flow cytometry analysis.

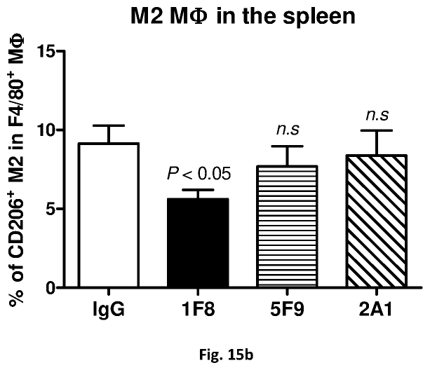

As shown in FIGS. 15a-15b, all of the tested CD47 antibodies (including 1F8) were able to induce polarization of macrophage in tumor-bearing mice.

### EXAMPLE 16. CD47 EXPRESSION PROFILE USING PDX SAMPLES OF VARIOUS HUMAN CANCER TYPES

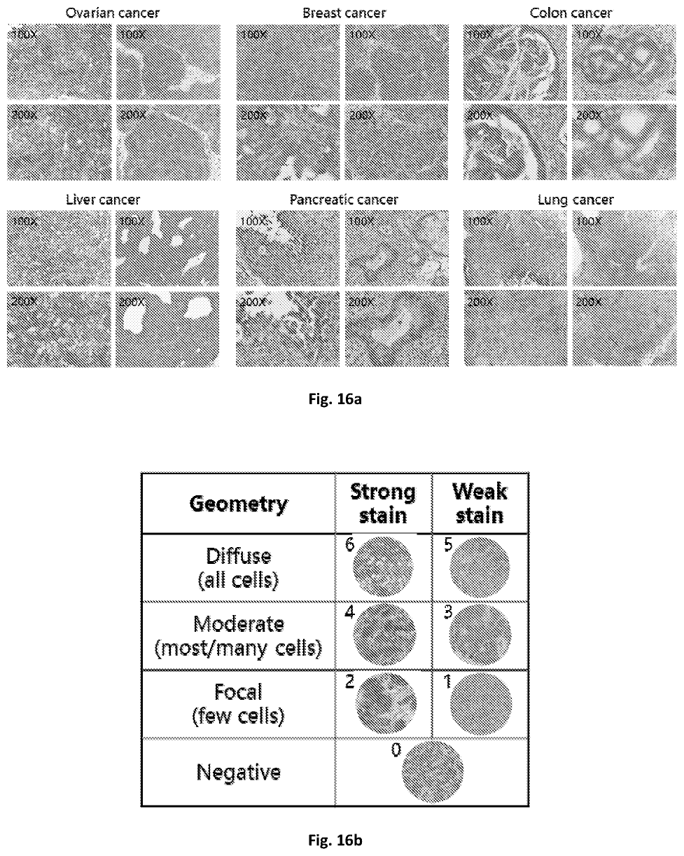

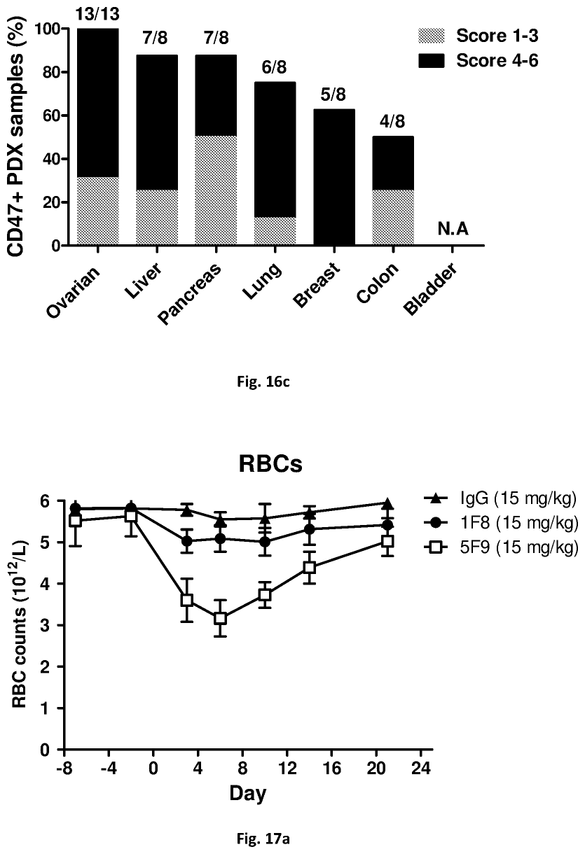

54 PDX samples (across 7 human cancer types) were analyzed for the expression of CD47 by immuno-histochemistry staining. The levels of CD47 staining in various PDX samples were scored by geometry and staining intensity. FIGS. 16a, 16b and 16c show the different expression levels of CD47 after the treatments with CD47 antibodies.

### EXAMPLE 17. SAFETY PHARMA STUDY (IN VIVO CYNO PK STUDIES)

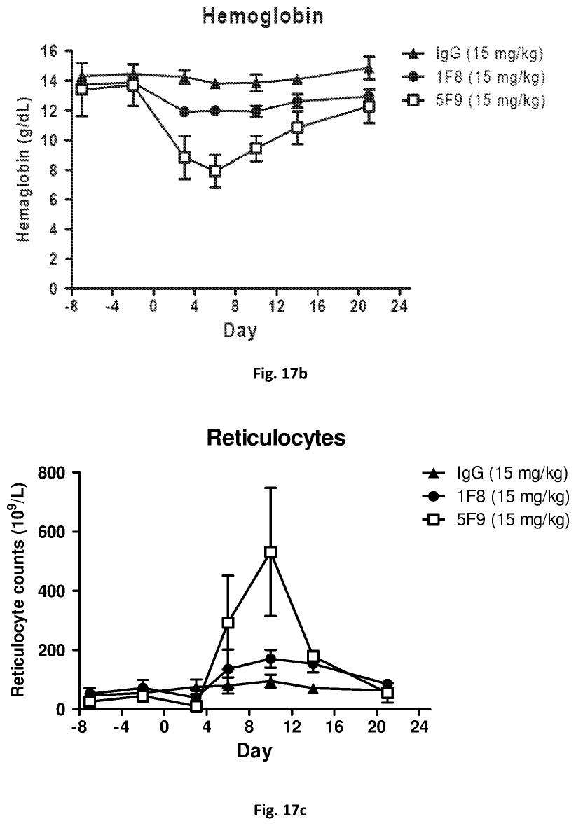

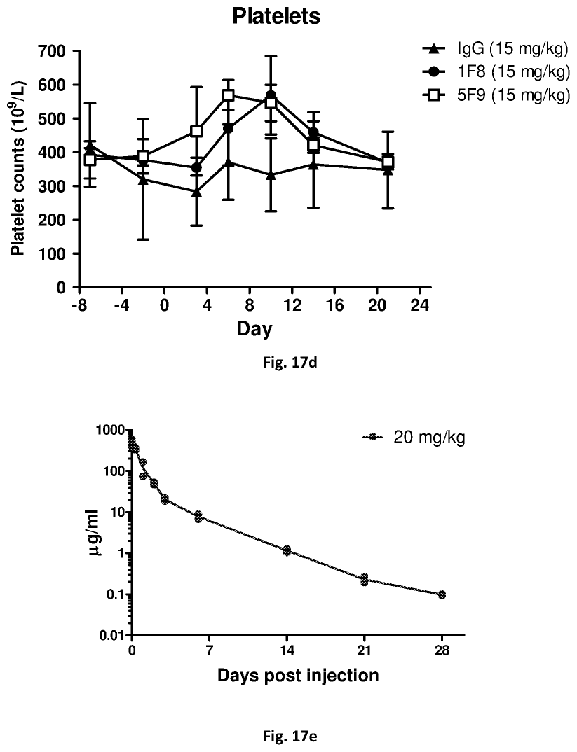

Naïve cyno monkeys were intravenously infused with vehicle (n=2), 1F8 (n=3, 15 mg/kg) and 5F9 (n=3, 15 mg/kg). Hematology (CBC) was analyzed within 24 hours after blood collection, twice before the injections and at 3, 6, 10, 14 and 21 days following the antibody administration. CBC parameters were examined including Erythrocyte count (RBC), Hemoglobin (HGB), Absolute Reticulocytes and Platelet Counts. The results are depicted in FIGS. 17a-17d and showed that 1F8 treatments did not affect the hematology parameters in cyno monkey.

Similarly, Naïve cyno monkeys (n=2) were intravenously injected with CD47 antibody 13H3 at a dose of 20 mg/kg. Their blood was collected by venipuncture into tubes with no anticoagulant at different time points. Serum level of the CD47 antibody 13H3 was measured by ELISA using CD47 protein as the coating reagent, followed by detection with an HRP-conjugated anti-human Kappa secondary antibody. Pharmacokinetic parameters in cyno monkeys were analyzed by Winolin and shown in FIG. 17e and the following table.

| T_{1/2} | Cₘₐₓ | AUC₀₋ₜ | AUC_{inf} | CL |
|---|---|---|---|---|
| (h) | (μg/ml) | (day*ug/ml) | (day*ug/ml) | (ml/hr/kg) |
| 145.2 ± | 536.4 ± | 10692.1 ± | 10712.5 ± | 1.880 ± |
| 10.8 | 63.9 | 1300.9 | 1298.4 | 0.228 |

### Safety Pharm Study (Hematology) of Antibody 13H3 in Cynomolgus Monkey

Naïve cyno monkeys were intravenously infused with single dose or repeat dose (weekly dosing) of the anitibody 13H3 (20 mg/kg). Hematology (CBC) parameters were examined including Erythrocyte count (RBC), Hemoglobin (HGB), Platelet Counts and Lymphocyte Counts at the indicated time points following the antibody administration.

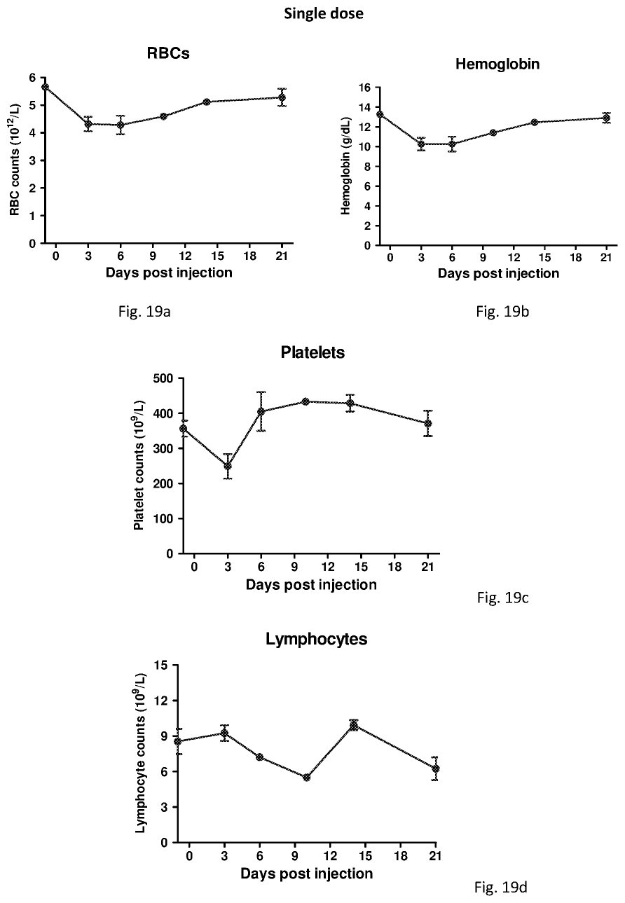

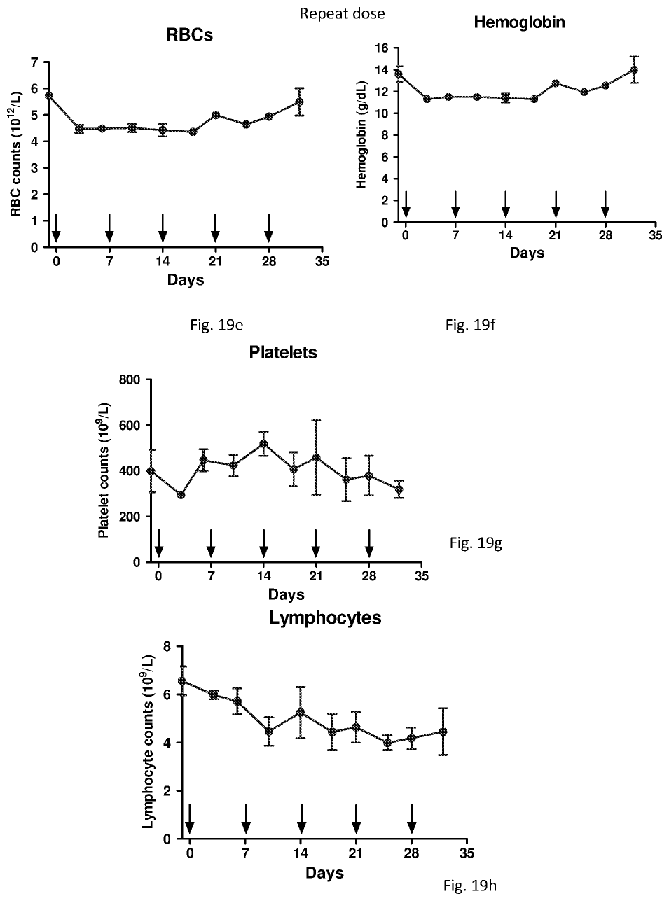

FIGS. 19a, 19b, 19c, 19d, 19e, 19f, 19g, and 19h show the effects of the CD47 antibody 13H3 on RBC congregation, hemoglobin, platelets, and lymphocytes.

### EXAMPLE 18. STRUCTURE OF ANTIBODY 1F8

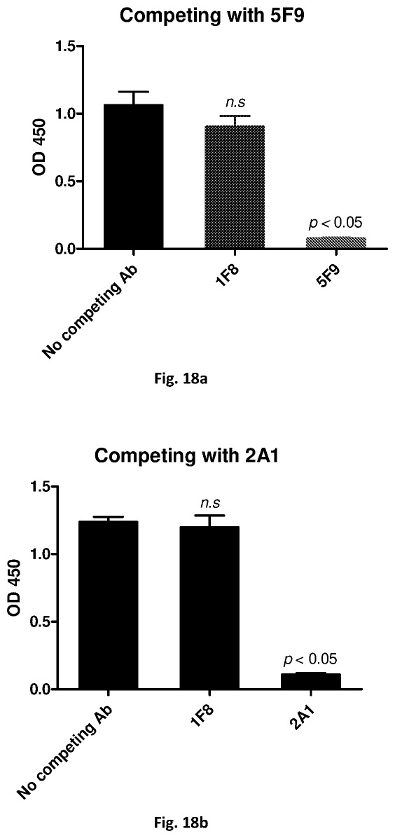

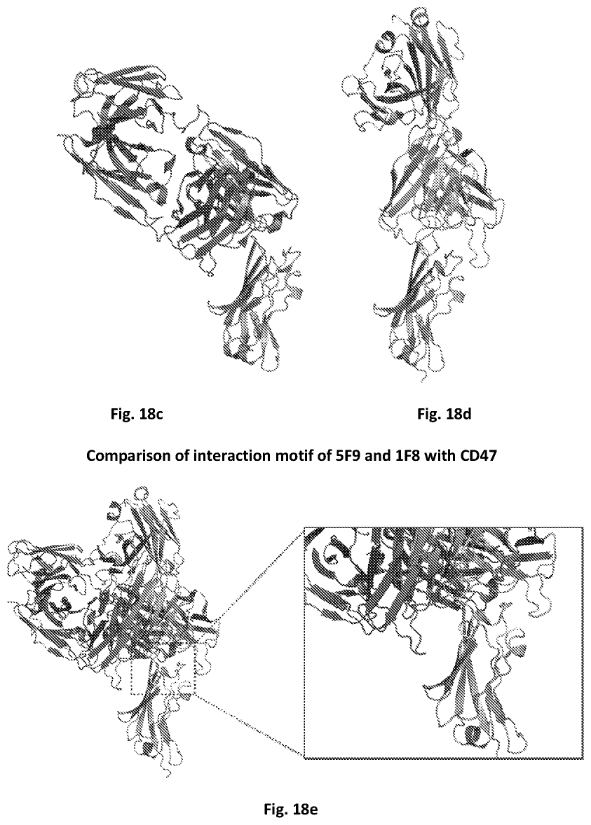

The epitope binning of CD47 antibodies was assessed by competition ELISA. CD47 ECD protein and first anti-CD47 antibody were pre-incubated and added to a biotinylated second anti-CD47 antibody detected by a Strptavidin-HRP antibody. If the first anti-CD47 antibody competed against the binding of CD47 ECD to the second antibody, both antibodies were placed in same or overlapping epitope bins. If not, they were placed in non-overlapping epitope bins. The results depicted in FIGS. 18a and 18b show that CD47 antibody of this invention 1F8 has a different epitope than those of reference antibodies 5F9 and 2A1.

FIG. 18c shows the crystal structure of reference Ab 5F9 (upper part) in complex with human CD47-ECD (green) as reported in the literature (See, e.g., J. Clin. Investigation, 126, 7: 2610-2620).

FIG. 18d shows the crystal structure of 1F8-Fab (upper part) in complex with human CD47-ECD (green). The complex structure of CD47-1F8 Fab adopts straighter head to head orientation, unlike the complex structures of CD47-SIRPα and CD47-5F9 diabody presenting tilted head to head orientation. The 1F8 epitope on CD47 is discontinuous and extensive which includes residues L3, V25, T26, N27, M28, E29, A30, 031, T34, E35, Y37, A53, L54, L74, K75, G76, T99, E100, L101, T102 and R103, of which L3, N27, E29, Q31, T34, E35, Y37, A53, T99, E100, L101, T102 and R103 are involved in the interactions with SIRPα, explaining the antagonistic properties of 1F8. The complex structure also reveals VH domain of 1F8 forms 8 hydrogen bonds and 4 salt bridges to CD47 and VL domain of 1F8 forms 8 hydrogen bonds to CD47 as well.

Unlike published CD47-IgV/antibody or SIRPa complex structures, the 1F8 antibody binds mostly different epitopes of the target although all are binding in the similar head-to-head orientation. The 1F8 epitope on CD47 is conformationally discontinuous and includes a TNMEAQ loop (residues 26-31), T34, E35, L74, and an LTR hinge (residues 101 -103) of CD47. Many hydrogen bond interactions are formed between side chains of antibody residues and CD47 main chain oxygen atoms. A salt bridge is also formed between R103 of 1F8 and E35 of CD47. Several Van der Waals contacts are also observed which are critical to keep appropriate orientation. The VH domain of antibody 1F8 is primarily involved in binding to the T34, E35 and the LTR hinge (residues 101-103) of CD47, while the VK domain interact with the TNMEAQ loop (residues 26-31) and L74. These epitopes on CD47 are different from that in 5F9 antibody and SIRPa. Structural analysis suggest that two long loops (residues 26-38 and 52-59) of the 1F8 antibody help it bind to CD47 in a nearly vertical orientation which may lead to the antibody to be separated in such a way that CD47 on adjacent cells could not be bridged by the antibody, thereby preventing most of blood cell hemaglutination.

FIG. 18e shows the comparison of interaction of 5F9 and 1F8 with CD47.

Superposition of reference antibody 5F9/CD47 complex structure on complex structure of 1F8/CD47 reveals that binding orientation of CD47 is very different between these two complexes. Although both antibodies have head-to-head binding orientation, CD47 is rotated horizontally by about 180 degree. The structure of 1F8/CD47 complex has CD47 N-terminal pyroglutamate near light chain loop residues 61-64, while 5F9 has CD47 N-term among 3 heavy chain loops of W52, N32 and W101. In antibody 1F8, the heavy chain residues Trp33 and Arg103 form van der Waals contact and a salt bridge with Leu101 and Glu35 of CD47, respectively. At the same position, antibody 5F9's residue Tyr101 point towards N-term of CD47 through a van der Waals contact and Arg102 forms a hydrogen bond with Glu104 of CD47. Antibody 1F8's loop residues Asn31, Trp33, and hinge residues Arg53 and Asp56 form inter-domain hydrogen bonds net, then Asn31 and Arg53 form hydrogen bonds with main chain of Leu101 and Thr34 in CD47. At the same interface, 5F9 does not appear to make interaction, except residue Tyr 52 forms a van der Waals contact with Leu3 on CD47. The hinge (residue 52-56) is 3 residues shorted than that of 1F8 (residues 52-59). In light chain, both Fab 1F8 and 5F9 have several important hydrogen bond interactions with CD47 from the loop (V29 -Y38 in 1F8 and V152-Y158 in 5F9). Residues Y97 and Y98 in 1F8 “push” the loop (residues 26-38) away, and the latter formed 2 hydrogen bonds between 1F8 and CD47, namely between Arg34 of 1F8 and main chain of Leu74 on CD47, and between Arg36 of 1F8 and main chain of Thr26 on CD47. However, 5F9's residues Gly218 and Ser219 (which correspond to Tyr97 and Tyr98 in 1F8) cause the loop (residues 149-158) in 5F9 to form 3 hydrogen bonds with CD47 (at Asn157-Lys39, Tyr159-G1u104 and Lys177-Thr99,). Also like that in heavy chain, the loop (residues 149-158) in 5F9 is about 3 residues shorter than that in 1F8 (residues 26-38). These relative longer loops in 1F8 mainly contribute to the binding orientation of the CD47.

### Example 19. CD47 Antibody 34C5

To generate anti-human CD47 antibodies, different strains of 6-8-week mice including BALB/C, C57/BL6 or SJL mice were immunized with recombinant human CD47 extracellular domain protein for several rounds. After immunization, mice with sufficient titres of anti-CD47 IgG were boosted with the same antigen followed by fusion. The hybridoma supernatants were tested for direct binding with human CD47 ECD protein and competition of SIRPα binding to CD47 by ELISA screening. Through a series of screening assays, 34C5 was selected for the humanization and further in vitro characterization according to the assays described above.

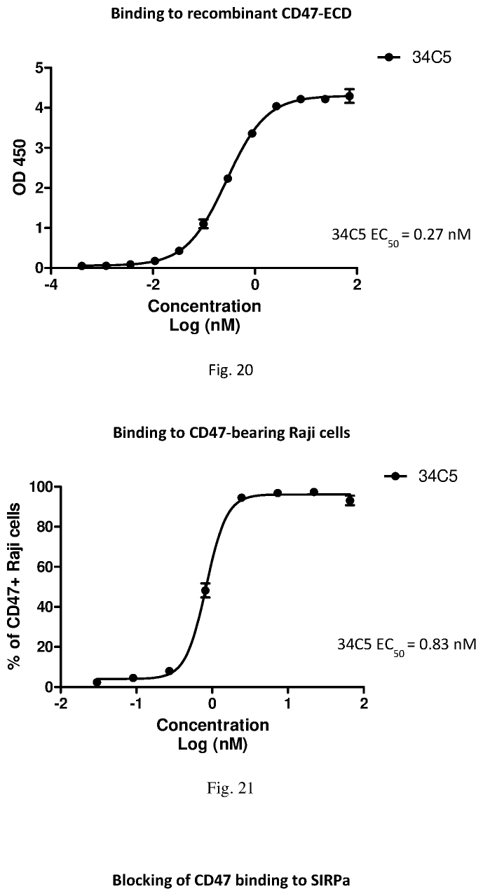

FIG. 20 and FIG. 21 show strong binding affinity of 34C5 to recombinant CD47-ECD (with an EC₅₀ of 0.27 nM) and to CD47-bearing Raji cells (with an EC₅₀ of 0.83 nM), respectively.

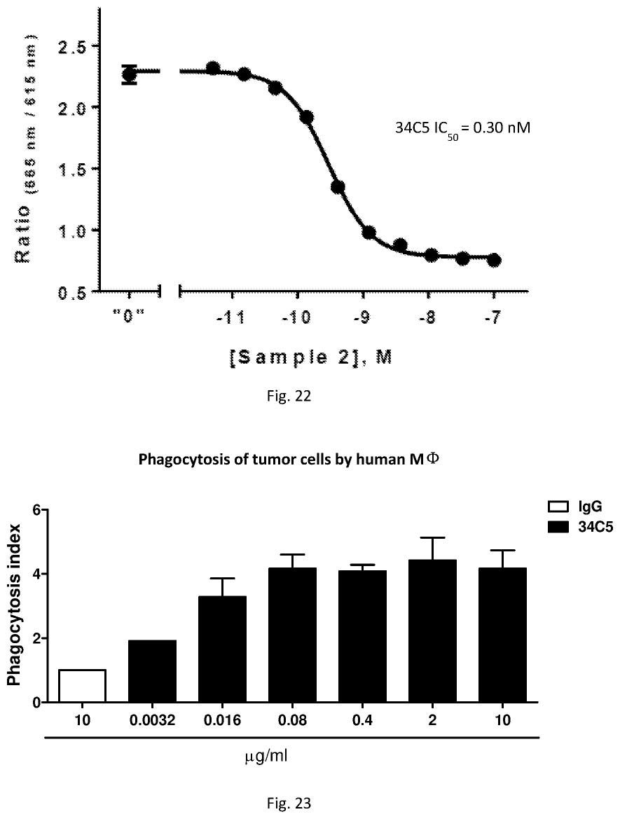

FIG. 22 shows that 34C5 was able to effectively block CD47 binding to SIRPα, with an EC₅₀ of 0.30 nM.

FIG. 23 shows that the antibody 34C5 promoted phagocystosis of tumor cells by human MCP.

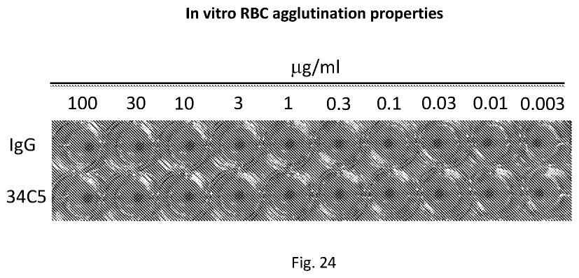

FIG. 24 shows the antibody 34C5 did not cause in vitro RBC agglutination.

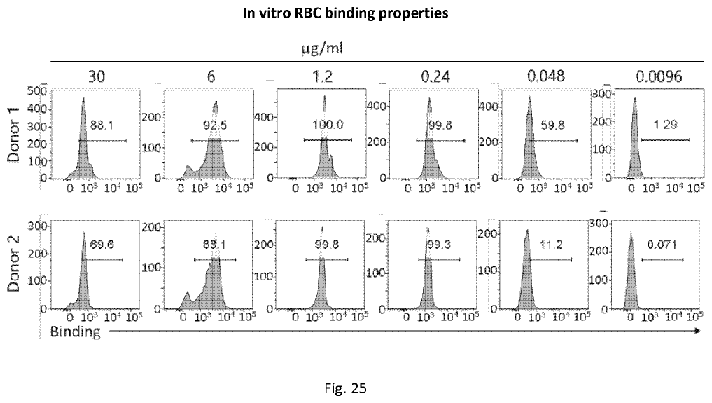

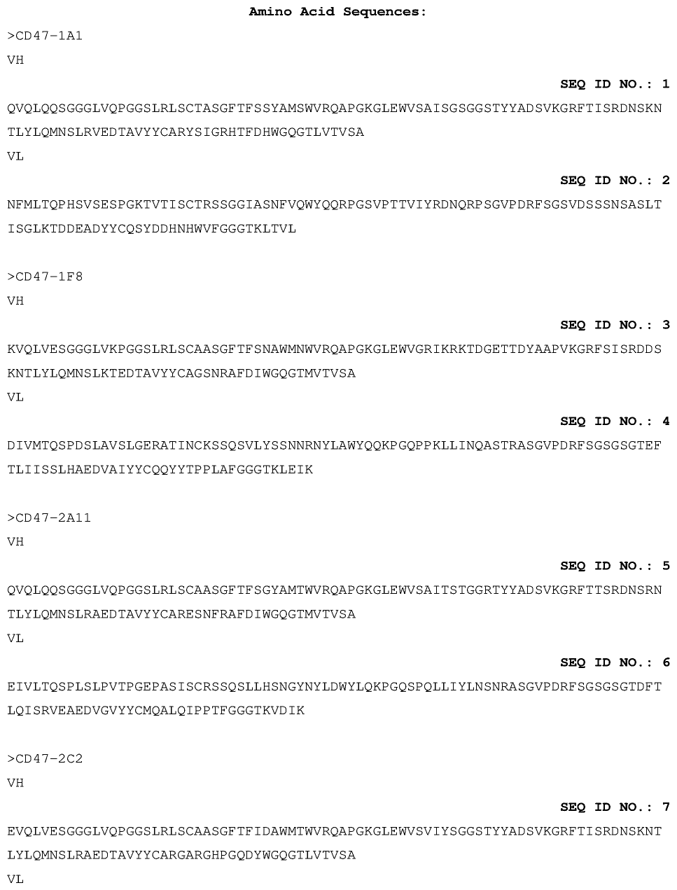

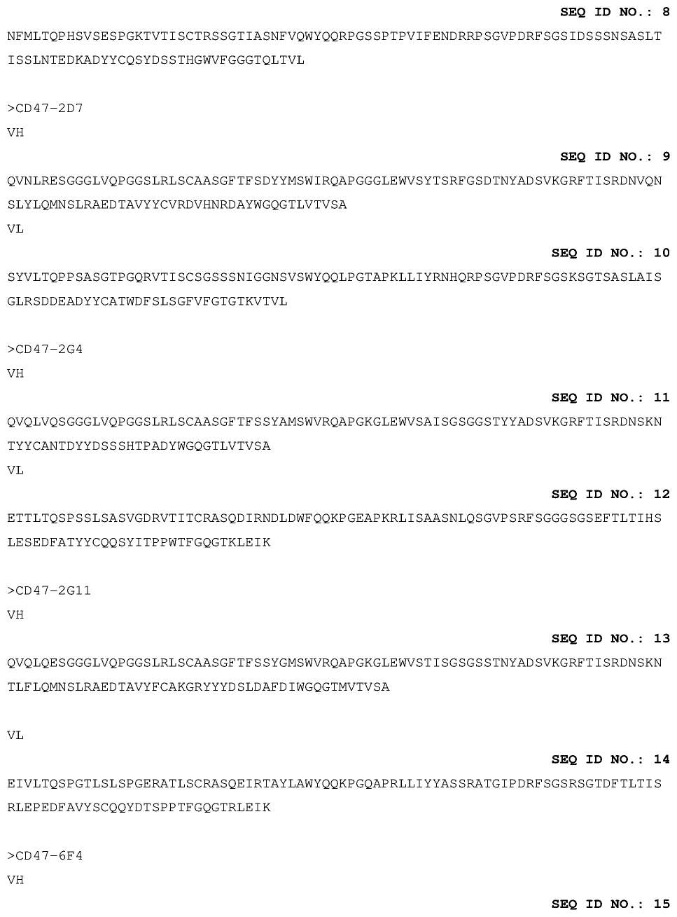

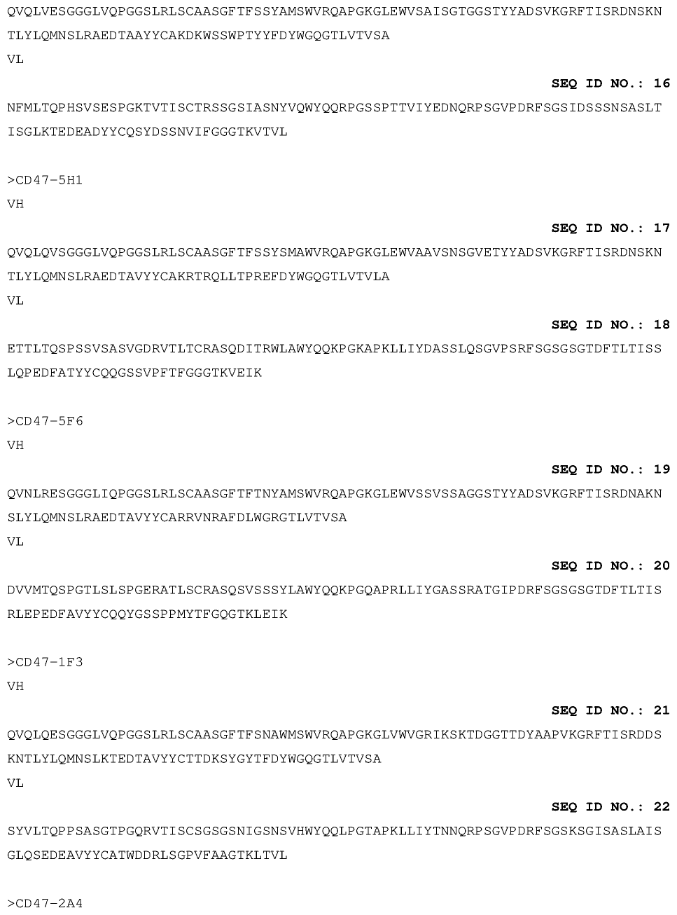

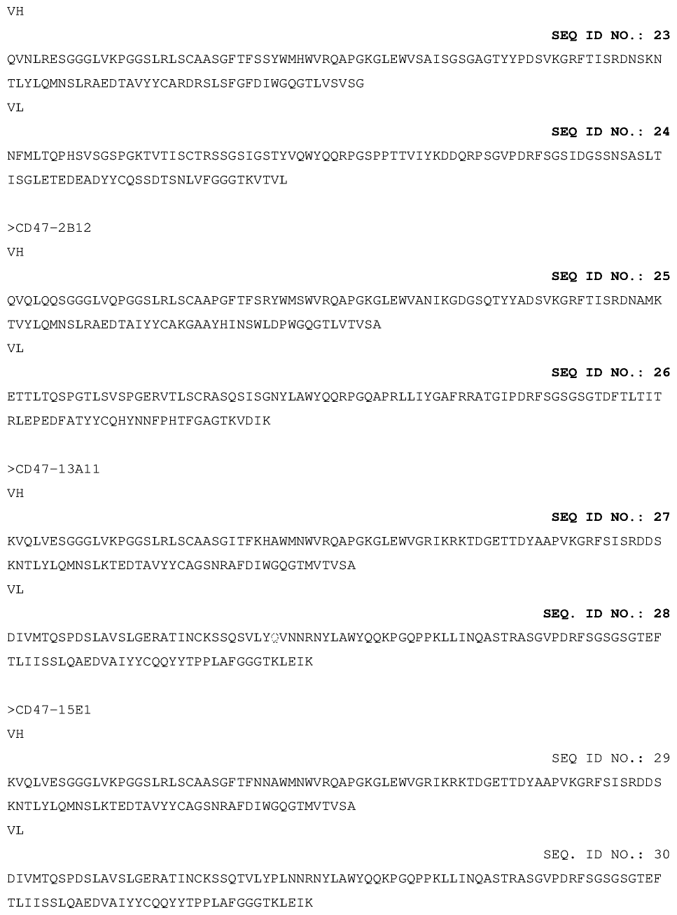

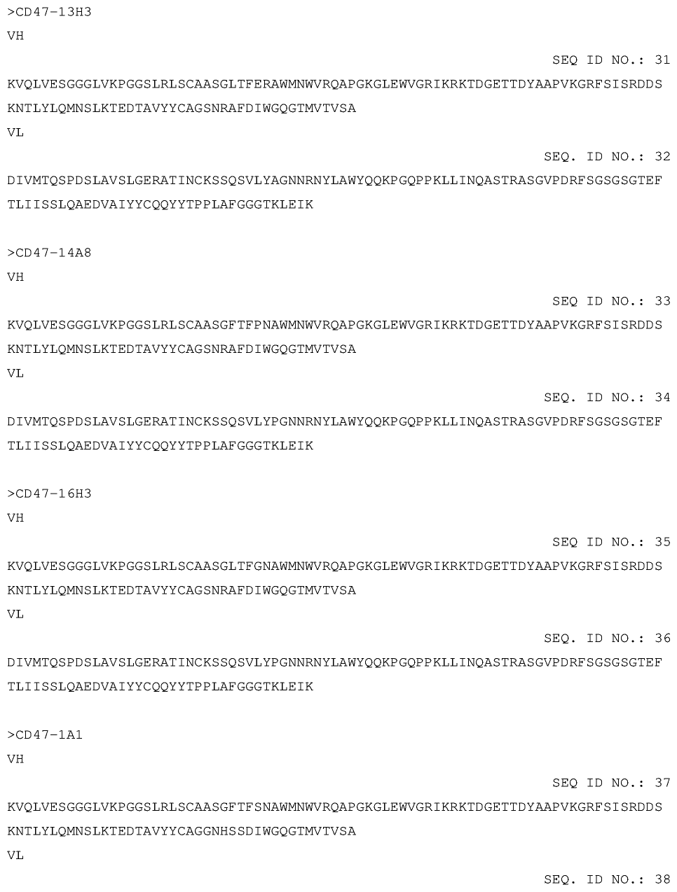

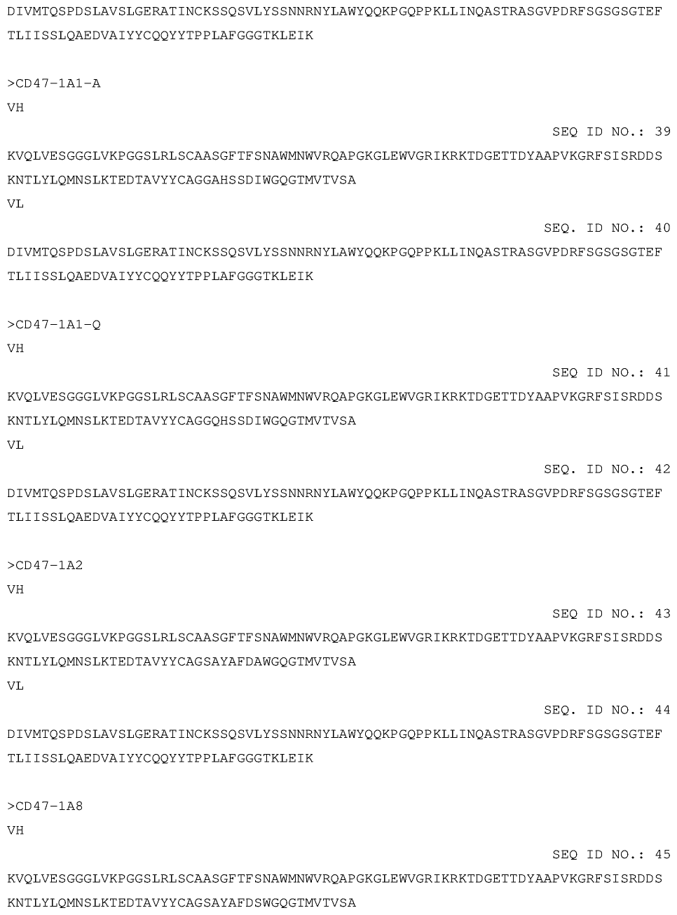

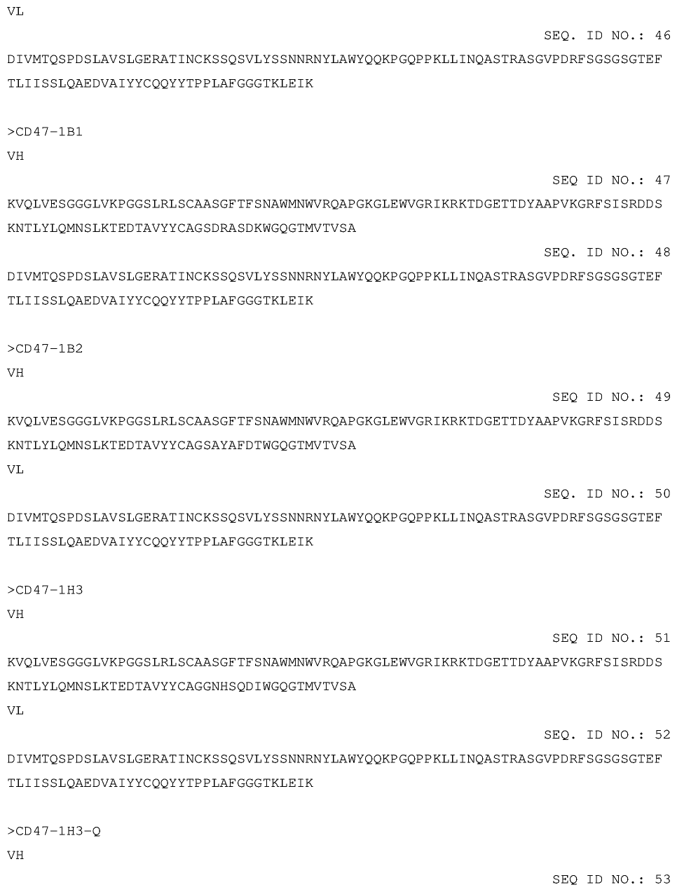

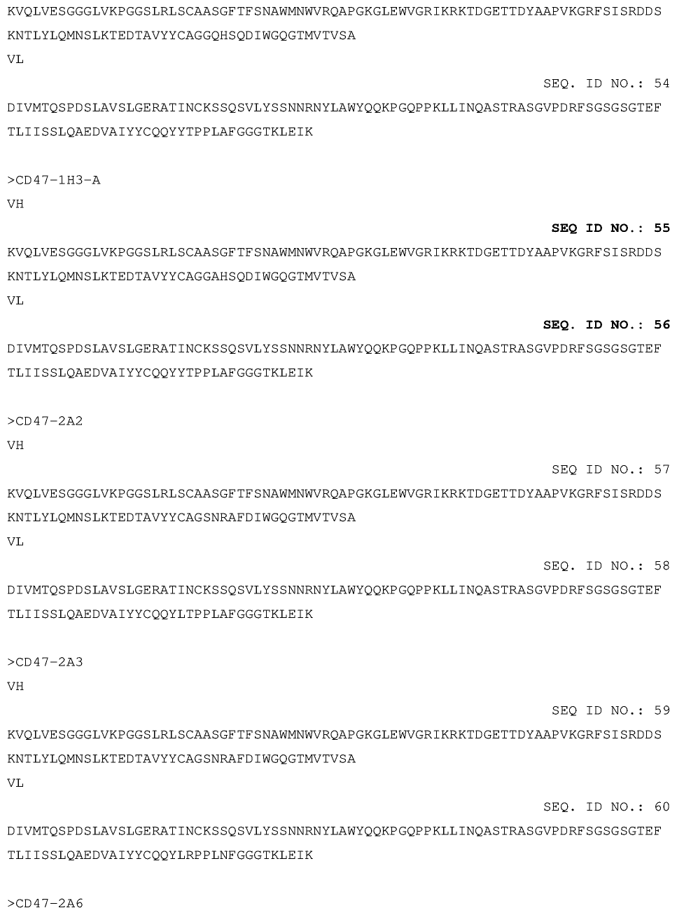

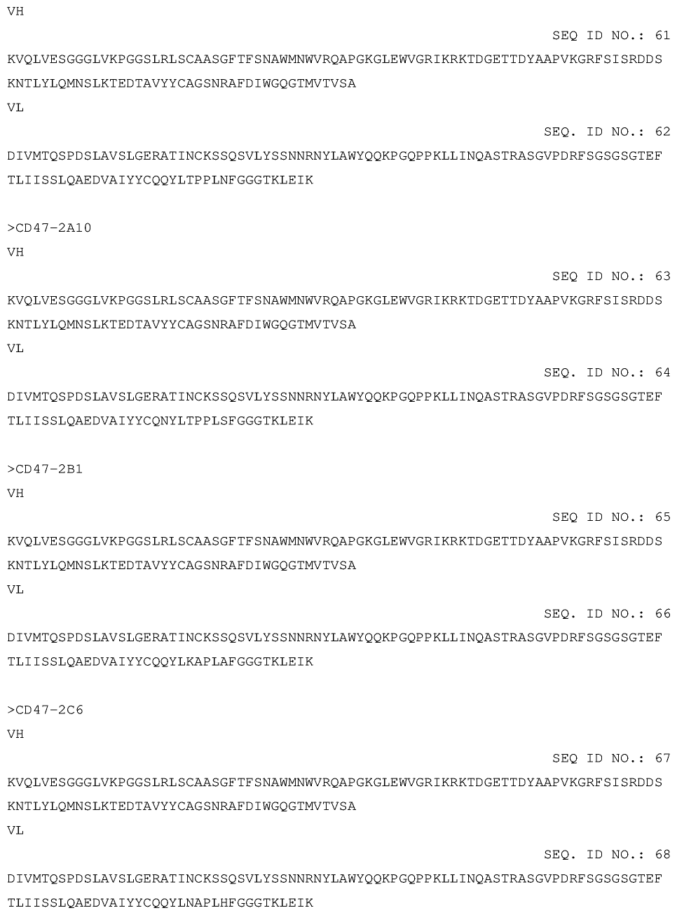

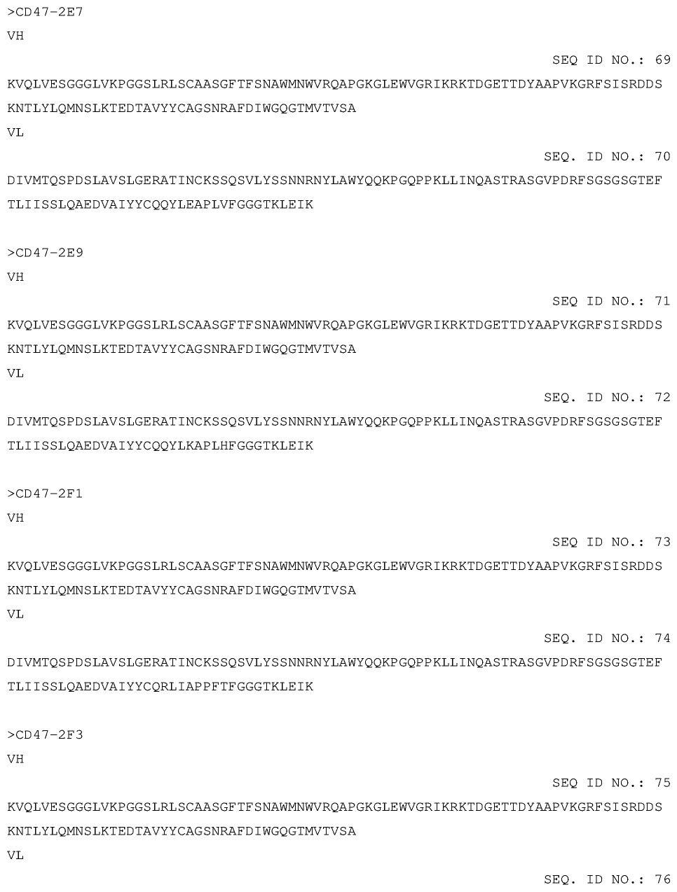

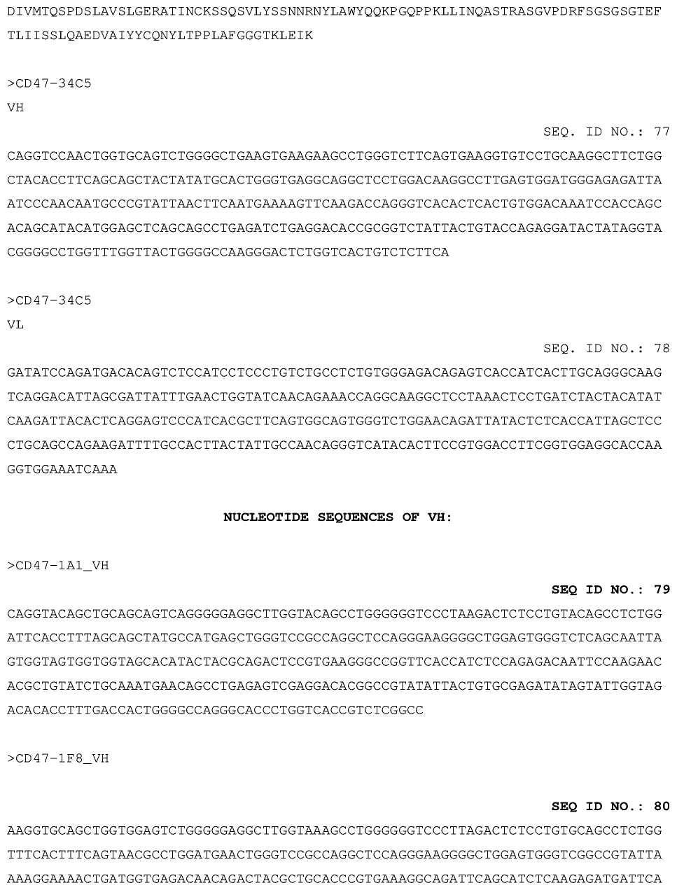

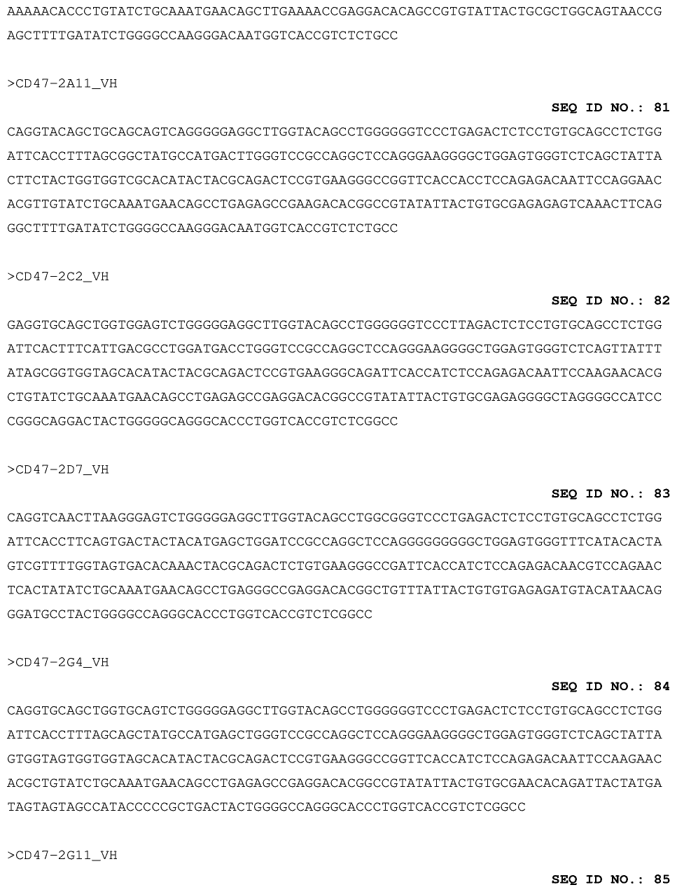

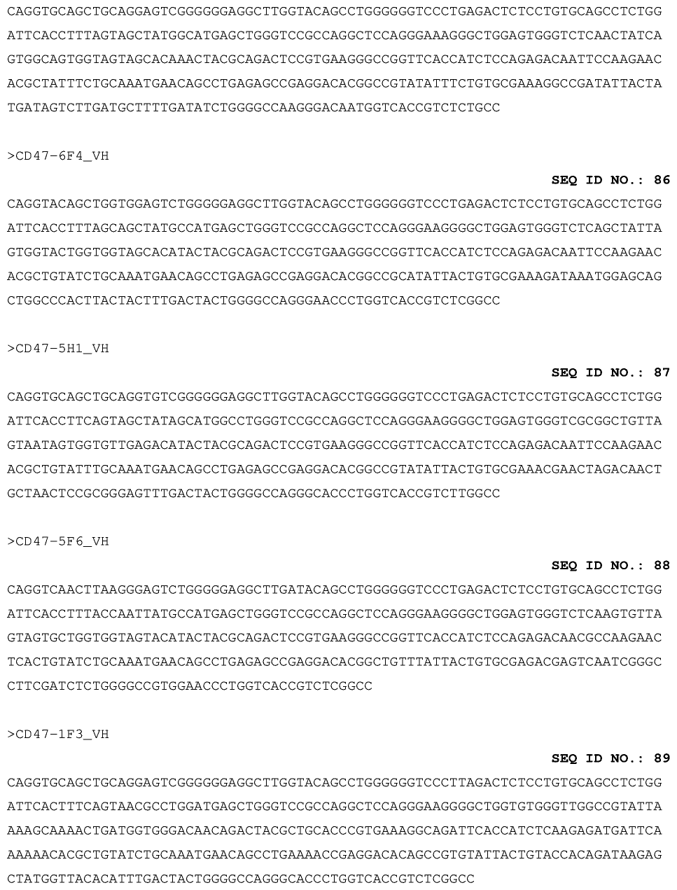

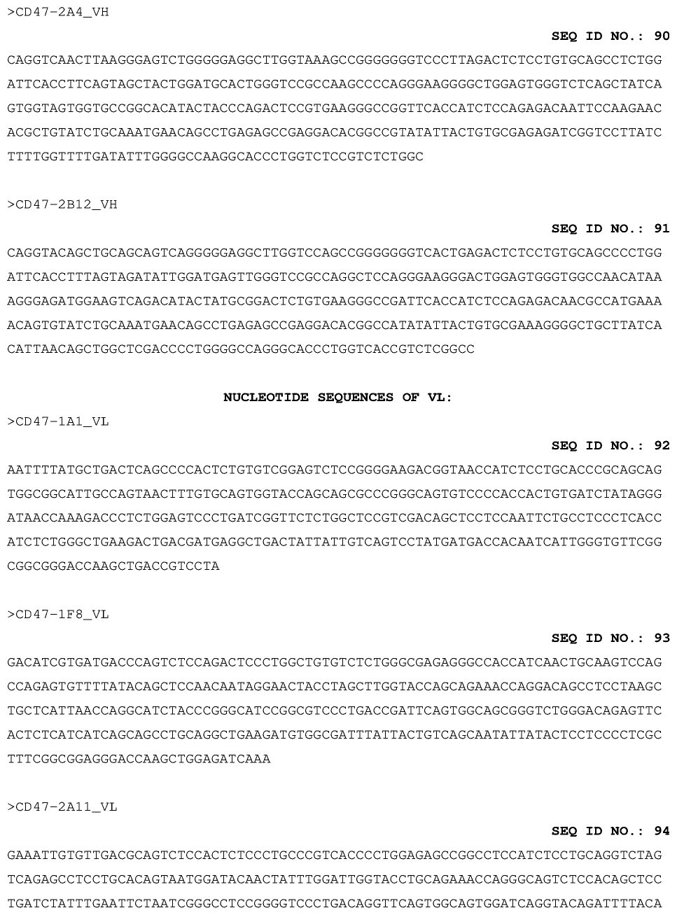

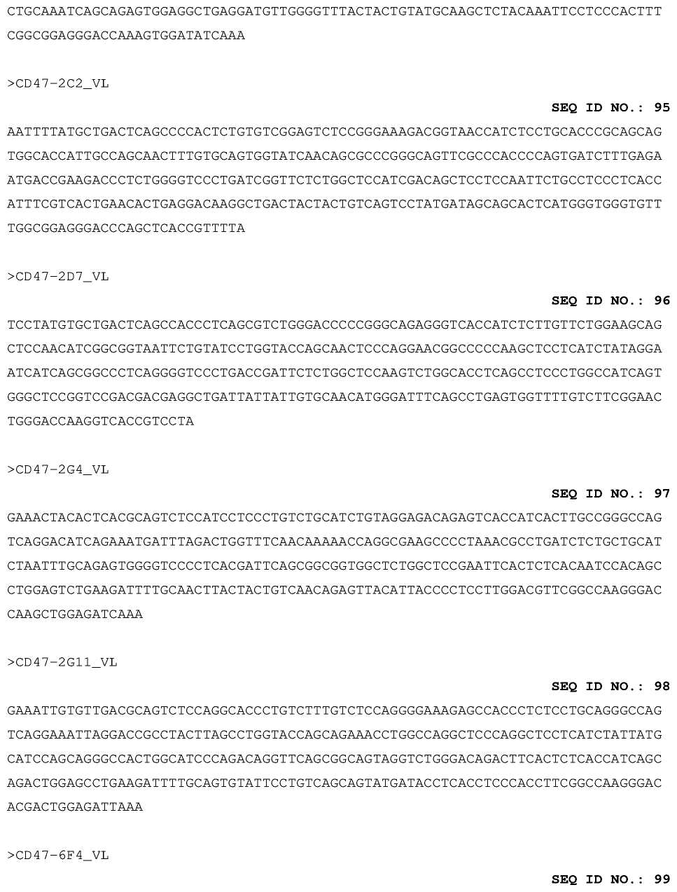

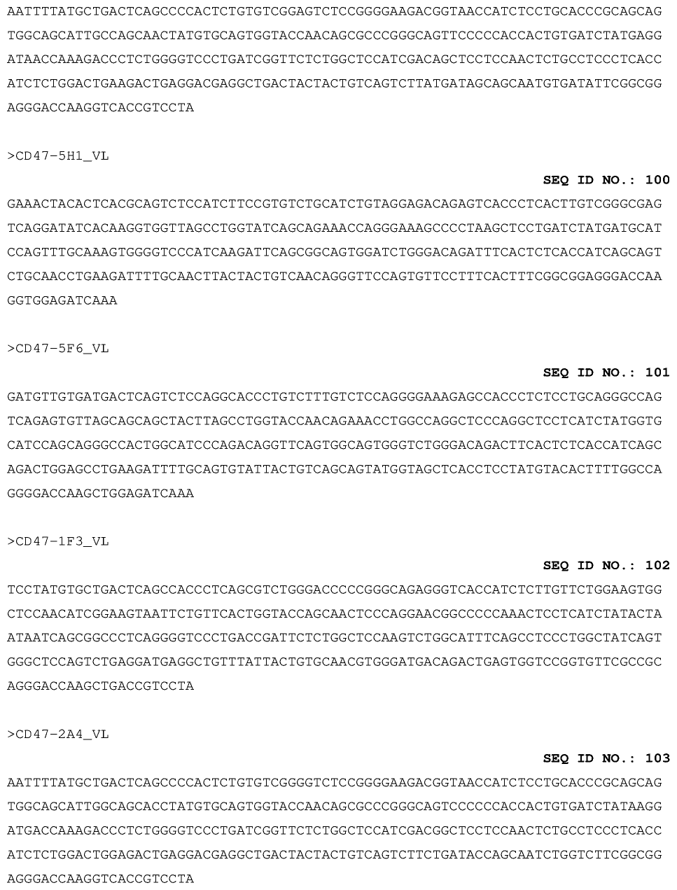

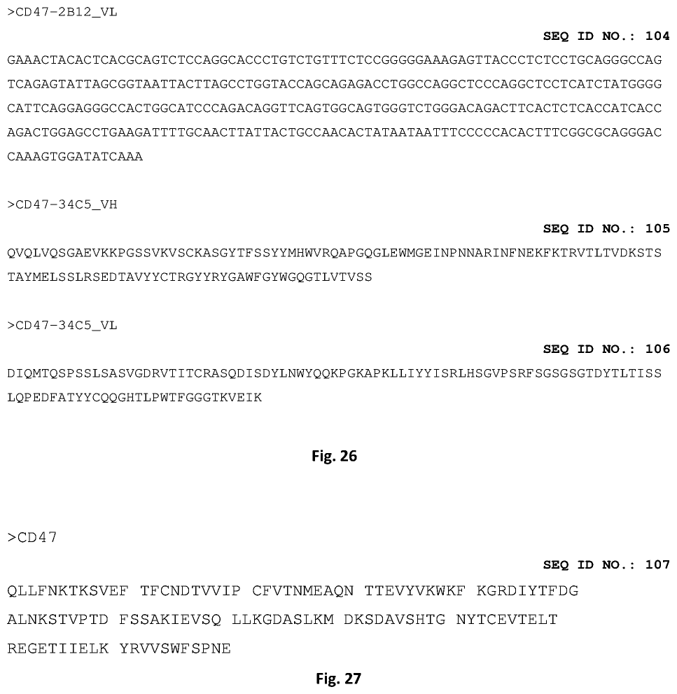

FIG. 25 shows the antibody 34C5 decrease its binding to RBC with the decreasing concentration of this antibody.

### EXAMPLE 20. GLYCOSYLATION OF RBC

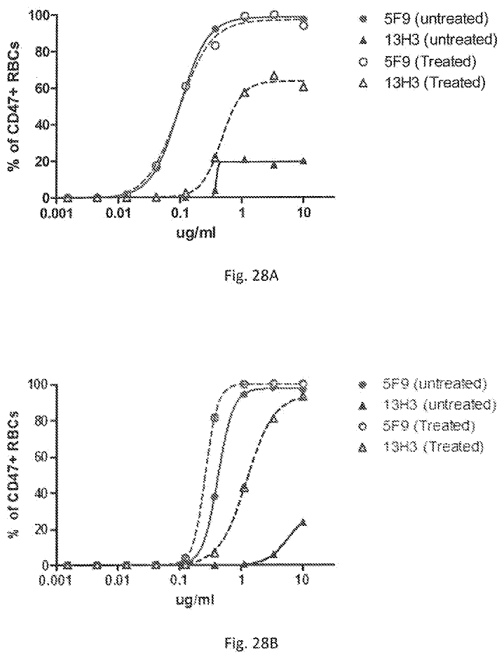

Purified human RBCs were pre-incubated with or without PNGase F for 1 hr or 6 hrs followed by the addition of different concentrations of 13H3 or 5F9. The binding of 13H3 or 5F9 against PNGase treated or untreated RBCs was then analyzed by flow cytometry. The results as depicted in FIGS. 28A and 28B show that deglycosylation of RBCs after the PNGase treatment restored the binding of 13H3 while did not affect the binding of control CD47 antibody 5F9, and the extension of the PNGase treatment resulted in a more sufficient deglycosylation of RBCs which led to an increased level of binding of 13H3 nearly to reach the full receptor occupancy.

1. A pharmaceutical composition, comprising a glycosylation agent, an antibody that binds to human CD47, and a pharmaceutically acceptable carrier.

2. The pharmaceutical composition of claim 1, wherein the antibody is a monoclonal antibody, a bispecific antibody, or a fusion protein comprising a monoclonal or bispecific antibody and a cytokine.

3. The pharmaceutical composition of claim 2, wherein the monoclonal antibody comprises
a variable heavy (VH) chain sequence that is at least 95% identical to an amino acid sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, and SEQ ID NO: 77; and
a variable light (VL) chain sequence that is at least 95% identical to an amino acid sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, and SEQ ID NO: 78.

4. The pharmaceutical composition of claim 3, wherein the monoclonal antibody comprises a VH/VL pair and the VH/VL pair comprises VH and VL chain sequences at least 95% identical to a pair of VH and VL amino acid sequences selected from the group consisting of SEQ ID NO: 1 and SEQ ID NO: 2, SEQ ID NO: 3 and SEQ ID NO: 4, SEQ ID NO: 5 and SEQ ID NO: 6, SEQ ID NO: 7 and SEQ ID NO: 8, SEQ ID NO: 9 and SEQ ID NO: 10, SEQ ID NO: 11 and SEQ ID NO: 12, SEQ ID NO: 13 and SEQ ID NO: 14, SEQ ID NO: 15 and SEQ ID NO: 16, SEQ ID NO: 17 and SEQ ID NO: 18, SEQ ID NO: 19 and SEQ ID NO: 20, SEQ ID NO: 21 and SEQ ID NO: 22, SEQ ID NO: 23 and SEQ ID NO: 24, SEQ ID NO: 25 and SEQ ID NO: 26, SEQ ID NO: 27 and SEQ ID NO: 28, SEQ ID NO: 29 and SEQ ID NO: 30, SEQ ID NO: 31 and SEQ ID NO: 32, SEQ ID NO: 33 and SEQ ID NO: 34, SEQ ID NO: 35 and SEQ ID NO: 36, SEQ ID NO: 37 and SEQ ID NO: 38, SEQ ID NO: 39 and SEQ ID NO: 40, SEQ ID NO: 41 and SEQ ID NO: 42, SEQ ID NO: 43 and SEQ ID NO: 44, SEQ ID NO: 45 and SEQ ID NO: 46, SEQ ID NO: 47 and SEQ ID NO: 48, SEQ ID NO: 49 and SEQ ID NO: 50, SEQ ID NO; 51 and SEQ ID NO: 52, SEQ ID NO: 53 and SEQ ID NO: 54, SEQ ID NO: 55 and SEQ ID NO: 56, SEQ ID NO: 57 and SEQ ID NO: 58, SEQ ID NO: 59 and SEQ ID NO: 60, SEQ ID NO: 61 and SEQ ID NO: 62, SEQ ID NO: 63 and SEQ ID NO: 64, SEQ ID NO: 65 and SEQ ID NO: 66, SEQ ID NO: 67 and SEQ ID NO: 68, SEQ ID NO: 69 and SEQ ID NO: 70, SEQ ID NO: 71 and SEQ ID NO: 72, SEQ ID NO: 73 and SEQ ID NO: 74, SEQ ID NO: 75 and SEQ ID NO: 76, and SEQ ID NO: 77 and SEQ ID NO: 78.

5. The pharmaceutical composition of claim 4, wherein the VH/VL pair comprises VH and VL chain sequences selected from the group consisting of SEQ ID NO: 1 and SEQ ID NO: 2, SEQ ID NO: 3 and SEQ ID NO: 4, SEQ ID NO: 5 and SEQ ID NO: 6, SEQ ID NO: 7 and SEQ ID NO: 8 , SEQ ID NO: 9 and SEQ ID NO: 10, SEQ ID NO: 11 and SEQ ID NO: 12, SEQ ID NO: 13 and SEQ ID NO: 14, SEQ ID NO: 15 and SEQ ID NO: 16, SEQ ID NO: 17 and SEQ ID NO: 18, SEQ ID NO: 19 and SEQ ID NO: 20, SEQ ID NO: 21 and SEQ ID NO: 22, SEQ ID NO: 23 and SEQ ID NO; 24, SEQ ID NO: 25 and SEQ ID NO; 26, SEQ ID NO: 27 and SEQ ID NO: 28, SEQ ID NO: 29 and SEQ ID NO: 30, SEQ ID NO: 31 and SEQ ID NO: 32, SEQ ID NO: 33 and SEQ ID NO: 34, SEQ ID NO: 35 and SEQ ID NO: 36, SEQ ID NO: 37 and SEQ ID NO: 38, SEQ ID NO: 39 and SEQ ID NO: 40, SEQ ID NO: 41 and SEQ ID NO: 42, SEQ ID NO: 43 and SEQ ID NO: 44, SEQ ID NO: 45 and SEQ ID NO: 46, SEQ ID NO: 47 and SEQ ID NO: 48, SEQ ID NO: 49 and SEQ ID NO: 50, SEQ ID NO: 51 and SEQ ID NO: 52, SEQ ID NO: 53 and SEQ ID NO: 54, SEQ ID NO: 55 and SEQ ID NO: 56, SEQ ID NO: 57 and SEQ ID NO: 58, SEQ ID NO: 59 and SEQ ID NO: 60, SEQ ID NO: 61 and SEQ ID NO: 62, SEQ ID NO: 63 and SEQ ID NO: 64, SEQ ID NO: 65 and SEQ ID NO: 66, SEQ ID NO: 67 and SEQ ID NO: 68, SEQ ID NO: 69 and SEO, ID NO: 70, SEQ ID NO: 71 and SEQ ID NO: 72, SEQ ID NO: 73 and SEQ ID NO: 74, SEQ ID NO: 75 and SEQ ID NO: 76, and SEQ ID NO: 77 and SEQ ID NO: 78.

6. The pharmaceutical composition of claim 2, wherein the monoclonal antibody or is chimeric or humanized.

7. The pharmaceutical composition of claim 2, wherein the bispecific antibody comprising a first arm and a second arm, the first arm comprises a first monoclonal antibody which binds human CD47, and the second arm comprise a second monoclonal antibody that does not bind human CD47.

8. The pharmaceutical composition of claim 7, wherein the second arm in the bispecific antibody binds to a cancer cell.

9. The pharmaceutical composition of claim 2, wherein the monoclonal or bispecific antibody exists in the form of a fusion protein that includes the monoclonal or bispecific antibody and a cytokine, with or without a linked between the monoclonal or bispecific antibody and the cytokine.

10. The pharmaceutical composition of claim 9, wherein the cytokine comprises a wild type or a variant of an immunoglobulin (Ig), a hemopoietic growth factor, an interferon, a tumor necrosis factor, an interleukin-17 receptor, or a monomeric glycoprotein.

11. The pharmaceutical composition of claim 10, wherein the cytokine is a wild type or a variant of the monomeric glycoprotein.

12. The fusion protein of claim 12, wherein the cytokine is a wild type or variant of granulocyte-macrophage colony-stimulating factor (GM-CSF).

13. The fusion protein of claim 9, wherein the monoclonal antibody is fused to the cytokine without a linker, or with a linker selected from the group consisting of (G4S)3, (G4S)6, (GS)9, IGD(F30), IGD(F64), IGD(R30), IGN(R64), IGD(R30-Cys), and 1GD(R64-Cys).

14. The pharmaceutical composition of claim 1, wherein the glycosylating gent comprises glycosyl group connected or bonded to a leaving group which is a halo, thioalkyl, imidate, acetate, phosphate, or O-pentenyl group.

15. An antibody that binds to human CD47, wherein the antibody binds to at least one epitope residue of CD47 near or at one of the glycosylation sites of the CD47 protein of red blood cells, resulting in low or no binding of the antibody to red blood cells due to a glycosylation site in red blood cells near the epitope residues Gln31 and Thr34 in CD47.

16. The antibody of claim 15, wherein the antibody binds to at least epitope residues Gln31 and Thr34 in CD47,

17. The antibody of claim 15, wherein the antibody is a monoclonal antibody which comprises:
a variable heavy (VH) chain sequence that is at least 95% identical to an amino acid sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, and SEQ ID NO: 77; and
a variable light (VL) chain sequence that is at least 95% identical to an amino acid sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, and SEQ ID NO: 78.

18. The antibody of claim 17, wherein monoclonal antibody comprises a VH/VL pair and the VH/VL pair comprises VH and VL chain sequences at least 95% identical to a pair of VH and VL amino acid sequences selected from the group consisting of SEQ ID NO: 1 and SEQ ID NO: 2, SEQ ID NO: 3 and SEQ ID NO: 4, SEQ ID NO: 5 and SEQ ID NO: 6, SEQ ID NO: 7 and SEQ ID NO: 8, SEQ ID NO: 9 and SEQ ID NO: 10, SEQ ID NO: 11 and SEQ ID NO: 12, SEQ ID NO: 13 and SEQ ID NO: 14, SEQ ID NO: 15 and SEQ ID NO: 16, SEQ ID NO: 17 and SEQ ID NO: 18, SEQ ID NO: 19 and SEQ ID NO: 20, SEQ ID NO: 21 and SEQ ID NO: 22, SEQ ID NO: 23 and SEQ ID NO: 24, SEQ ID NO: 25 and SEQ ID NO: 26, SEQ ID NO: 27 and SEQ ID NO: 28, SEQ ID NO: 29 and SEQ ID NO: 30, SEQ ID NO: 31 and SEQ ID NO: 32, SEQ ID NO: 33 and SEQ ID NO: 34, SEQ ID NO: 35 and SEQ ID NO: 36, SEQ ID NO: 37 and SEQ ID NO: 38, SEQ ID NO: 39 and SEQ ID NO: 40, SEQ ID NO: 41 and SEQ ID NO: 42, SEQ ID NO: 43 and SEQ ID NO: 44, SEQ ID NO: 45 and SEQ ID NO: 46, SEQ ID NO: 47 and SEQ ID NO: 48, SEQ ID NO: 49 and SEQ ID NO: 50, SEQ ID NO: 51 and SEQ ID NO: 52, SEQ ID NO: 53 and SEQ ID NO: 54, SEQ ID NO: 55 and SEQ ID NO: 56, SEQ ID NO: 57 and SEQ ID NO: 58, SEQ ID NO: 59 and SEQ ID NO: 60, SEQ ID NO: 61 and SEQ ID NO: 62, SEQ ID NO: 63 and SEQ ID NO: 64, SEQ ID NO: 65 and SEQ ID NO: 66, SEQ ID NO: 67 and SEQ ID NO: 68, SEQ ID NO: 69 and SEQ ID NO: 70, SEQ ID NO: 71 and SEQ ID NO: 72, SEQ ID NO: 73 and SEQ ID NO: 74, SEQ ID NO: 75 and SEQ ID NO: 76, and SEQ ID NO: 77 and SEQ ID NO: 78.

19. The pharmaceutical composition of claim 18, wherein the VH/VL pair comprises VH and VL chain sequences selected from the group consisting of SEQ ID NO: 1 and SEQ ID NO: 2, SEQ ID NO: 3 and SEQ ID NO: 4, SEQ ID NO: 5 and SEQ ID NO: 6, SEQ ID NO: 7 and SEQ ID NO: 8 , SEQ ID NO: 9 and SEQ ID NO: 10, SEQ ID NO: 11 and SEQ ID NO: 12, SEQ ID NO: 13 and SEQ ID NO: 14, SEQ ID NO: 15 and SEQ ID NO: 16, SEQ ID NO: 17 and SEQ ID NO: 18, SEQ ID NO: 19 and SEQ ID NO: 20, SEQ ID NO: 21 and SEQ ID NO: 22, SEQ ID NO: 23 and SEQ ID NO: 24, SEQ ID NO: 25 and SEQ ID NO: 26, SEQ ID NO: 27 and SEQ ID NO: 28, SEQ ID NO: 29 and SEQ ID NO: 30, SEQ ID NO: 31 and SEQ ID NO: 32, SEQ ID NO: 33 and SEQ ID NO: 34, SEQ ID NO: 35 and SEQ ID NO: 36, SEQ ID NO: 37 and SEQ ID NO: 38, SEQ ID NO: 39 and SEQ ID NO: 40, SEQ ID NO: 41 and SEQ ID NO: 42, SEQ ID NO: 43 and SEQ ID NO: 44, SEQ ID NO: 45 and SEQ ID NO: 46, SEQ ID NO: 47 and SEQ ID NO: 48, SEQ ID NO: 49 and SEQ ID NO: 50, SEQ ID NO: 51 and SEQ ID NO: 52, SEQ ID NO: 53 and SEQ ID NO: 54, SEQ ID NO: 55 and SEQ ID NO: 56, SEQ ID NO: 57 and SEQ ID NO: 58, SEQ ID NO: 59 and SEQ ID NO: 60, SEQ ID NO: 61 and SEQ ID NO: 62, SEQ ID NO: 63 and SEQ ID NO: 64, SEQ ID NO: 65 and SEQ ID NO: 66, SEQ ID NO: 67 and SEQ ID NO: 68, SEQ ID NO: 69 and SEQ ID NO: 70, SEQ ID NO: 71 and SEQ ID NO: 72, SEQ ID NO: 73 and SEQ ID NO: 74, SEQ ID NO: 75 and SEQ ID NO: 76, and SEQ ID NO: 77 and SEQ ID NO: 78.

20. The antibody of claim 16, wherein the antibody is a bispecific antibody comprising a first arm and a second arm, the first arm comprises a first monoclonal antibody which binds human CD47, the second arm comprise a second monoclonal antibody that does not bind human CD47.

21. The antibody of claim 20, wherein the first arm of the bispecific antibody comprises a VH/VL pair and the VH/VL pair comprises VH and VL chain sequences at least 95% identical to a pair of VH and VL amino acid sequences selected from the group consisting of SEQ ID NO: 1 and SEQ ID NO: 2, SEQ ID NO: 3 and SEQ ID NO: 4, SEQ ID NO: 5 and SEQ ID NO: 6, SEQ ID NO: 7 and SEQ ID NO: 8, SEQ ID NO: 9 and SEQ ID NO: 10, SEQ ID NO: 11 and SEQ ID NO: 12, SEQ ID NO: 13 and SEQ ID NO: 14, SEQ ID NO: 15 and SEQ ID NO: 16, SEQ ID NO: 17 and SEQ ID NO: 18, SEQ ID NO: 19 and SEQ ID NO: 20, SEQ ID NO: 21 and SEQ ID NO: 22, SEQ ID NO: 23 and SEQ ID NO: 24, SEQ ID NO: 25 and SEQ ID NO: 26, SEQ ID NO: 27 and SEQ ID NO: 28, SEQ ID NO: 29 and SEQ ID NO: 30, SEQ ID NO: 31 and SEQ ID NO: 32, SEQ ID NO: 33 and SEQ ID NO: 34, SEQ ID NO: 35 and SEQ ID NO: 36, SEQ ID NO: 37 and SEQ ID NO: 38, SEQ ID NO: 39 and SEQ ID NO: 40, SEQ ID NO: 41 and SEQ ID NO: 42, SEQ ID NO: 43 and SEQ ID NO: 44, SEQ ID NO: 45 and SEQ ID NO: 46, SEQ ID NO: 47 and SEQ ID NO: 48, SEQ ID NO: 49 and SEQ ID NO: 50, SEQ ID NO: 51 and SEQ ID NO: 52, SEQ ID NO: 53 and SEQ ID NO: 54, SEQ ID NO: 55 and SEQ ID NO: 56, SEQ ID NO: 57 and SEQ ID NO: 58, SEQ ID NO: 59 and SEQ ID NO: 60, SEQ ID NO: 61 and SEQ ID NO: 62, SEQ ID NO: 63 and SEQ ID NO: 64, SEQ ID NO: 65 and SEQ ID NO: 66, SEQ ID NO: 67 and SEQ ID NO: 68, SEQ ID NO: 69 and SEQ ID NO: 70, SEQ ID NO: 71 and SEQ ID NO: 72, SEQ ID NO: 73 and SEQ ID NO: 74, SEQ ID NO: 75 and SEQ ID NO: 76, and SEQ ID NO: 77 and SEQ ID NO: 78.

22. A method for treating a disease in a human subject in need thereof, comprising administering to the subject a therapeutically effective amount of a pharmaceutical composition of claim 1, wherein the disease is cancer, a fibrotic disease, a disease related to inhibition of phagocytosis, or a disease related to platelet aggregation.

23. The method of claim 22, wherein the cancer is selected from the group consisting of: ovarian cancer, colon cancer; breast cancer, lung cancer, head and neck cancer, bladder cancer, colorectal cancer, pancreatic cancer, non-Hodgkin's lymphoma, acute lymphocytic leukemia, chronic lymphocytic leukemia, acute myeloid leukemia, chronic myelogenous leukemia, hairy cell leukemia (HCL), T-cell prolymphocytic leukemia (T-PLL), large granular lymphocytic leukemia, adult T-cell leukemia, multiple myeloma, melanoma, leiomyoma, leiomyosarcoma, glioma, glioblastoma, myelomas, monocytic leukemias, B-cell derived leukemias, T-cell derived leukemias, B-cell derived lymphomas, T-cell derived lymphomas, endometrial cancer, kidney cancer, melanoma, prostate cancer, thyroid cancer, cervical cancer, gastric cancer, liver cancer; and solid tumors; the fibrotic disease is selected from the group consisting of: myocardial infarction, angina, osteoarthritis, pulmonary fibrosis, asthma, cystic fibrosis, bronchitis, and asthma; the disease related to inhibition of phagocytosis is a cardiovascular disease; the disease related to platelet aggregation is Glanzmann Thrombasthenia, prolonged bleeding time, immune thrombocytopenia (ITP), von Willebrand disease (vWD).

24. The method of claim 22, wherein the cardiovascular disease is selected from the group consisting of atherosclerosis, stroke, hypertensive heart disease, rheumatic heart disease, cardiomyopathy, heart arrhythmia, congenital heart disease, valvular heart disease, carditis, aortic aneurysms, peripheral artery disease, and venous thrombosis.

25. A method for increasing the selectivity of binding of a monoclonal or bispecific antibody to human CD47 while decreasing the binding of the antibody to red blood cells, comprising subjecting the red blood cells with a glycosylation agent before or concurrently with subjecting the human CD47 to the antibody.

26. The method of claim 25, the red blood cells are subjected to the glycosylation agent before human CD47 is subjected to the CD47 antibody.

27. The method of claim 25, further comprising cleglycosylation of the red blood cells.

28. The method of claim 27, wherein the deglysosylation step is carried out with a PNGase.

29. The method of claim 25, wherein the CD47 antibody is an isolated monoclonal antibody which comprises:
a variable heavy (VH) chain sequence that is at least 95% identical to an amino acid sequence selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, and SEQ ID NO: 77; and
a variable light (VL) chain sequence that is at least 95% identical to an amino acid sequence selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, and SEQ ID NO: 78.

30. The method of any of claim 29, wherein the CD47 antibody comprises a VH/VL pair, wherein the VH/VL pair comprises VH and VL chain sequences at least 95% identical to a pair of VH and VL amino acid sequences selected from the group consisting of SEQ ID NO: 1 and SEQ ID NO: 2, SEQ ID NO: 3 and SEQ ID NO: 4, SEQ ID NO: 5 and SEQ ID NO: 6, SEQ ID NO: 7 and SEQ ID NO: 8, SEQ ID NO: 9 and SEQ ID NO: 10, SEQ ID NO: 11 and SEQ ID NO: 12, SEQ ID NO: 13 and SEQ ID NO: 14, SEQ ID NO: 15 and SEQ ID NO: 16, SEQ ID NO: 17 and SEQ ID NO: 18, SEQ ID NO: 19 and SEQ ID NO: 20, SEQ ID NO: 21 and SEQ ID NO: 22, SEQ ID NO: 23 and SEQ ID NO: 24, SEQ ID NO: 25 and SEQ ID NO: 26, SEQ ID NO: 27 and SEQ ID NO: 28, SEQ ID NO: 29 and SEQ ID NO: 30, SEQ ID NO: 31 and SEQ ID NO; 32, SEQ ID NO: 33 and SEQ ID NO: 34, SEQ ID NO: 35 and SEQ ID NO: 36, SEQ ID NO: 37 and SEQ ID NO: 38, SEQ ID NO: 39 and SEQ ID NO: 40, SEQ ID NO: 41 and SEQ ID NO: 42, SEQ ID NO: 43 and SEQ ID NO: 44, SEQ ID NO: 45 and SEQ ID NO: 46, SEQ ID NO: 47 and SEQ ID NO: 48, SEQ ID NO: 49 and SEQ ID NO: 50, SEQ ID NO: 51 and SEQ ID NO: 52, SEQ ID NO: 53 and SEQ ID NO: 54, SEQ ID NO: 55 and SEQ ID NO: 56, SEQ ID NO: 57 and SEQ ID NO: 58, SEQ ID NO: 59 and SEQ ID NO: 60, SEQ ID NO: 61 and SEQ ID NO: 62, SEQ ID NO: 63 and SEQ ID NO: 64, SEQ ID NO: 65 and SEQ ID NO: 66, SEQ ID NO: 67 and SEQ ID NO: 68, SEQ ID NO: 69 and SEQ ID NO: 70, SEQ ID NO: 71 and SEQ ID NO: 72, SEQ ID NO: 73 and SEQ ID NO: 74, SEQ ID NO: 75 and SEQ ID NO: 76, and SEQ ID NO: 77 and SEQ ID NO: 78.

31. The method of claim 30, wherein the CD47 antibody comprises a VH/VL pair, wherein the VH/VL pair comprises VH and VL chain sequences selected from the group consisting of SEQ ID NO: 1 and SEQ ID NO: 2, SEQ ID NO: 3 and SEQ ID NO: 4, SEQ ID NO: 5 and SEQ ID NO: 6, SEQ ID NO: 7 and SEQ ID NO: 8 , SEQ ID NO: 9 and SEQ ID NO: 10, SEQ ID NO: 11 and SEQ ID NO: 12, SEQ ID NO: 13 and SEQ ID NO: 14, SEQ ID NO: 15 and SEQ ID NO: 16, SEQ ID NO: 17 and SEQ ID NO: 18, SEQ ID NO: 19 and SEQ ID NO: 20, SEQ ID NO: 21 and SEQ ID NO: 22, SEQ ID NO: 23 and SEQ ID NO: 24, SEQ ID NO: 25 and SEQ ID NO: 26, SEQ ID NO; 27 and SEQ ID NO: 28, SEQ ID NO; 29 and SEQ ID NO: 30, SEQ ID NO: 31 and SEQ ID NO: 32, SEQ ID NO: 33 and SEQ ID NO: 34, SEQ ID NO: 35 and SEQ ID NO: 36, SEQ ID NO: 37 and SEQ ID NO: 38, SEQ ID NO: 39 and SEQ ID NO: 40, SEQ ID NO: 41 and SEQ ID NO: 42, SEQ ID NO: 43 and SEQ ID NO: 44, SEQ ID NO: 45 and SEQ ID NO: 46, SEQ ID NO: 47 and SEQ ID NO: 48, SEQ ID NO: 49 and SEQ ID NO: 50, SEQ ID NO: 51 and SEQ ID NO: 52, SEQ ID NO: 53 and SEQ ID NO: 54, SEQ ID NO: 55 and SEQ ID NO: 56, SEQ ID NO: 57 and SEQ ID NO: 58, SEQ ID NO: 59 and SEQ ID NO: 60, SEQ ID NO: 61 and SEQ ID NO: 62, SEQ ID NO: 63 and SEQ ID NO: 64, SEQ ID NO: 65 and SEQ ID NO: 66, SEQ ID NO: 67 and SEQ ID NO: 68, SEQ ID NO: 69 and SEQ ID NO: 70, SEQ ID NO: 71 and SEQ ID NO: 72, SEQ ID NO: 73 and SEQ ID NO: 74, SEQ ID NO: 75 and SEQ ID NO: 76, and SEQ ID NO: 77 and SEQ ID NO: 78.

32. The method of claim 25, wherein the CD47 antibody is chimeric or humanized.

33. The method of claim 25, wherein the CD47 antibody is bispecific and comprises a first arm and a second arm, the first arm comprises a first monoclonal antibody as described in any of claims 24-30 and binds human CD47, and the second arm comprise a second monoclonal antibody that does not bind human CD47.

34. The method of claim 33, wherein the second arm binds to a cancer cell.

35. A method for binding a CD47 antibody to human CD47 with low or no binding the CD47 antibody to red blood cells, comprising glycosylating the red blood cells with a glycosylation agent.

36. The method of claim 35, wherein the red blood cells are glycosylated at one or more N-linked glycosylation sites.

37. The method of claim 36, wherein the red blood cells have at least a glycosylation site near the epitope residues Gln31 and Thr34 in CD47.
